SEQUENCE LISTING (90 sequences)

SEQ ID NO: 1 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLASMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLDSMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 5 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 6 - protein, 495 aa, synthetic construct
KFTIVFPANQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 7 - protein, 495 aa, synthetic construct
KFTIVFPHNAKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 8 - protein, 495 aa, synthetic construct
KFTIVFPHNRKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 9 - protein, 495 aa, synthetic construct
KFTIVFPHNKKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 10 - protein, 517 aa, synthetic construct
MLSYLIFALVVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 11 - protein, 501 aa, synthetic construct
KIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 12 - protein, 512 aa, synthetic construct
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 13 - protein, 496 aa, synthetic construct
KFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 14 - protein, 523 aa, synthetic construct
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 15 - protein, 502 aa, synthetic construct
KITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 16 - protein, 511 aa, synthetic construct
MTPAFILCMLLAGSSWAKFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 17 - protein, 494 aa, synthetic construct
KFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 18 - protein, 512 aa, synthetic construct
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 19 - protein, 495 aa, synthetic construct
KFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 20 - protein, 513 aa, synthetic construct
MLVLYLLLSLLALGAQCKFTIVFPHNQKGNWKNVPANYQYCPSSSDLNWHNGLIGTSLQVKMPKSHKAIQADGWMCHAAKWVTTCDFRWYGPKYVTHSIKSMIPTVDQCKESIAQTKQGTWLNPGFPPQSCGYASVTDAEAVIVKATPHQVLVDEYTGEWVDSQFPTGKCNKDICPTVHNSTTWHSDYKVTGLCDANLISMDITFFSEDGKLTSLGKEGTGFRSNYFAYENGDKACRMQYCKHWGVRLPSGVWFEMADKDIYNDAKFPDCPEGSSIAAPSQTSVDVSLIQDVERILDYSLCQETWSKIRAHLPISPVDLSYLSPKNPGTGPAFTIINGTLKYFETRYIRVDIAGPIIPQMRGVISGTTTERELWTDWYPYEDVEIGPNGVLKTATGYKFPLYMIGHGMLDSDLHISSKAQVFEHPHIQDAASQLPDDETLFFGDTGLSKNPIELVEGWFSGWKSTIASFFFIIGLVIGLYLVLRIGIALCIKCRVQEKRPKIYTDVEMNRLDR

SEQ ID NO: 21 - protein, 496 aa, synthetic construct
KFTIVFPHNQKGNWKNVPANYQYCPSSSDLNWHNGLIGTSLQVKMPKSHKAIQADGWMCHAAKWVTTCDFRWYGPKYVTHSIKSMIPTVDQCKESIAQTKQGTWLNPGFPPQSCGYASVTDAEAVIVKATPHQVLVDEYTGEWVDSQFPTGKCNKDICPTVHNSTTWHSDYKVTGLCDANLISMDITFFSEDGKLTSLGKEGTGFRSNYFAYENGDKACRMQYCKHWGVRLPSGVWFEMADKDIYNDAKFPDCPEGSSIAAPSQTSVDVSLIQDVERILDYSLCQETWSKIRAHLPISPVDLSYLSPKNPGTGPAFTIINGTLKYFETRYIRVDIAGPIIPQMRGVISGTTTERELWTDWYPYEDVEIGPNGVLKTATGYKFPLYMIGHGMLDSDLHISSKAQVFEHPHIQDAASQLPDDETLFFGDTGLSKNPIELVEGWFSGWKSTIASFFFIIGLVIGLYLVLRIGIALCIKCRVQEKRPKIYTDVEMNRLDR

SEQ ID NO: 22 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLDSMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 23 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 24 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLDSMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 25 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 26 - protein, 330 aa, synthetic construct [sequence omitted]

SEQ ID NO: 27 - protein, 325 aa, synthetic construct
STKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 326 aa, synthetic construct
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 240 aa, synthetic construct
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 30 - protein, 7 aa, synthetic construct
GYPFTSY

SEQ ID NO: 31 - protein, 6 aa, synthetic construct
DPNSGD

SEQ ID NO: 32 - protein, 12 aa, synthetic construct
SPYYSNDNSMDY

SEQ ID NO: 33 - protein, 11 aa, synthetic construct
RASQSIGTSIH

SEQ ID NO: 34 - protein, 7 aa, synthetic construct
YASESIS

SEQ ID NO: 35 - protein, 9 aa, synthetic construct
QQSNSWPTT

SEQ ID NO: 36 - protein, 5 aa, synthetic construct
SYWIH

SEQ ID NO: 37 - protein, 17 aa, synthetic construct
RIDPNSGDTKYNEKFKN

SEQ ID NO: 38 - protein, 8 aa, synthetic construct
GYPFTSYW

SEQ ID NO: 39 - protein, 8 aa, synthetic construct
IDPNSGDT

SEQ ID NO: 40 - protein, 14 aa, synthetic construct
ARSPYYSNDNSMDY

SEQ ID NO: 41 - protein, 6 aa, synthetic construct
QSIGTS

SEQ ID NO: 42 - protein, 121 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSS

SEQ ID NO: 43 - protein, 108 aa, synthetic construct
DILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKR

SEQ ID NO: 44 - protein, 249 aa, synthetic construct
DILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSS

SEQ ID NO: 45 - protein, 249 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKR

SEQ ID NO: 46 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 47 - protein, 210 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLYK

SEQ ID NO: 48 - protein, 7 aa, synthetic construct
RYTFTDY

SEQ ID NO: 49 - protein, 6 aa, synthetic construct
YPYNGG

SEQ ID NO: 50 - protein, 12 aa, synthetic construct
DHRYNEGVSFDY

SEQ ID NO: 51 - protein, 15 aa, synthetic construct
RASESVDGFGNSFMN

SEQ ID NO: 52 - protein, 7 aa, synthetic construct
LASNLES

SEQ ID NO: 53 - protein, 9 aa, synthetic construct
QQNNEDPYT

SEQ ID NO: 54 - protein, 5 aa, synthetic construct
DYNLH

SEQ ID NO: 55 - protein, 17 aa, synthetic construct
FIYPYNGGTGYNQKFKN

SEQ ID NO: 56 - protein, 8 aa, synthetic construct
RYTFTDYN

SEQ ID NO: 57 - protein, 8 aa, synthetic construct
IYPYNGGT

SEQ ID NO: 58 - protein, 14 aa, synthetic construct
ARDHRYNEGVSFDY

SEQ ID NO: 59 - protein, 10 aa, synthetic construct
ESVDGFGNSF

SEQ ID NO: 60 - protein, 121 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 61 - protein, 112 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKR

SEQ ID NO: 62 - protein, 253 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKRGGGGSGGGGSGGGGSGGGGSEVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 63 - protein, 253 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSSGGGGSGGGGSGGGGSGGGGSNIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKR

SEQ ID NO: 64 - protein, 5 aa, synthetic construct
  REPEAT 1..5: note = Sequence is present n times, wherein n is 1, 2, 3, 4, or5
GGGGS

SEQ ID NO: 65 - protein, 16 aa, synthetic construct
MKCLLYLAFLFIGVNC

SEQ ID NO: 66 - protein, 16 aa, synthetic construct
MLSYLIFALVVSPILG

SEQ ID NO: 67 - protein, 16 aa, synthetic construct
MLRLFLFCFLALGAHS

SEQ ID NO: 68 - protein, 21 aa, synthetic construct
MKMKMVIAGLILCIGILPAIG

SEQ ID NO: 69 - protein, 17 aa, synthetic construct
MTPAFILCMLLAGSSWA

SEQ ID NO: 70 - protein, 17 aa, synthetic construct
MNFLLLTFIVLPLCSHA

SEQ ID NO: 71 - protein, 17 aa, synthetic construct
MLVLYLLLSLLALGAQC

SEQ ID NO: 72 - protein, 20 aa, synthetic construct
GGGGSGGGGSGGGGSGGGGS

SEQ ID NO: 73 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 74 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 75 - protein, 244 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 76 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 77 - protein, 15 aa, synthetic construct
GGGGSGGGGSGGGGS

SEQ ID NO: 78 - protein, 110 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTS

SEQ ID NO: 79 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 80 - protein, 248 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 81 - protein, 248 aa, synthetic construct
SEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTS

SEQ ID NO: 82 - protein, 232 aa, synthetic construct
EPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 83 - protein, 40 aa, synthetic construct
KIEVMYPPPYLDNEKSNGTIIHVKGKHLCPSPLFPGPSKP

SEQ ID NO: 84 - protein, 27 aa, synthetic construct
FWVLVVVGGVLACYSLLVTVAFIIFWV

SEQ ID NO: 85 - protein, 8 aa, synthetic construct
NRVRQGYS

SEQ ID NO: 86 - protein, 55 aa, synthetic construct
FVPVFLPAKPTTTPAPRPPTPAPTIASQPLSLRPEACRPAAGGAVHTRGLDFACD

SEQ ID NO: 87 - protein, 28 aa, synthetic construct
IYIWAPLAGTCGVLLLSLVITLYCNHRN

SEQ ID NO: 88 - protein, 11 aa, synthetic construct
GGTETSQVAPA

SEQ ID NO: 89 - protein, 609 aa, synthetic construct
METDTLLLWVLLLWVPGSTGDSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 90 - protein, 508 aa, synthetic construct
MALPVTALLLPLALLLHAARPGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKSGLDFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0234664 A1
(43) Pub. Date: Aug. 13, 2026

(54) PHARMACEUTICAL COMPOSITIONS VIRAL PARTICLES AND METHODS OF USING THE SAME

(71) Applicant: INTERIUS BIOTHERAPEUTICS, INC., Philadelphia, PA (US)

(72) Inventors: Muneeswara Babu Medi, Chalfont, PA (US); Timothy D. Culp, North Wales, PA (US); Richard W. Peluso, Lansdale, PA (US)

(21) Appl. No.: 19/469,909

(22) PCT Filed: Mar. 28, 2024
(86) PCT No.: PCT/US2024/021858

§ 371 Date: Sep. 26, 2025

Related U.S. Application Data
(60) Provisional application No. 63/492,664, filed on Mar. 28, 2023.

(51) Int. Cl. C12N 15/86; A61K 47/02; A61K 47/26; A61K 47/42; A61K 48/00; C07K 16/28

(52) U.S. Cl. CPC C12N 15/86; A61K 47/02; A61K 47/26; A61K 47/42; A61K 48/005; C07K 16/2803; C07K 2317/565; C12N 2740/15043; C12N 2740/15045; C12N 2760/20222

(57) ABSTRACT

Provided for herein are pharmaceutical compositions comprising a virus, such as lentiviruses, including pseudotyped lentiviruses and uses thereof. Also provided for herein are methods of using the pharmaceutical compositions.

### RELATED APPLICATIONS

This application claims the benefit of U.S. Provisional Application Ser. No. 63/492,664 filed Mar. 28, 2023, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Mar. 11, 2024, is named “INH-025WO_SL” and is 97,721 bytes in size.

### FIELD

Embodiments provided herein relate to viral vectors, and pharmaceutical compositions comprising the same.

### BACKGROUND

The stabilization of purified lentiviral particles physically and functionally requires an optimal formulation composition. Consideration must be given to pH, ionic strength and salt concentration, as well as stabilizing excipients during the processing, freezing, thawing, handling, and storage for optimal shelf-life. Lentiviral vectors have previously been formulated for mostly ex vivo use, which involves a single freeze-thaw cycle. The development of lentiviral vectors for direct in vivo administration that requires two freeze thaw cycles requires a formulation that stabilizes the product throughout this process. This is critical, as in vivo administration requires the use of appropriate formulation excipients that are safe for human administration with optimal pH and osmolality to minimize aggregation of viral particles while maintaining the functional titer. Pharmaceutical compositions comprising viral particles must have a higher ionic strength to minimize viral particle aggregation, and include a stabilizing excipient to preserve functional titer of viral particles through multiple freeze-thaw cycles and potential hold times during processing, after thawing, and prior to dose administration. The present disclosure addresses those needs and others.

### BRIEF SUMMARY

The disclosed embodiments are directed towards viral vectors, specifically comprising a VSV-G polypeptide, and pharmaceutical compositions that contain viruses, and particles thereof, that are specific for VSV-G.

In some embodiments, a pharmaceutical composition is provided comprising a lentiviral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, the lentiviral vector is present at a concentration of about 1.0E⁺⁰⁵ to about 1.0E⁺⁰⁹ transducing units (TUs)/mL.

In some embodiments, the pharmaceutically acceptable buffer is a histidine buffer.

In some embodiments, the histidine buffer is present at a concentration of about 10 mM to about 30 mM.

In some embodiments, the pharmaceutical composition has a pH of about 6.5 to about 7.5.

In some embodiments, the pharmaceutically acceptable buffer is a histidine buffer and the pharmaceutical composition has a pH of about 7.0.

In some embodiments, the pharmaceutically acceptable salt is sodium chloride.

In some embodiments, the sodium chloride is present at a concentration of about 150 mM.

In some embodiments, the sugar is sucrose.

In some embodiments, the sucrose is present at about 4% (w/v).

In some embodiments, the protein excipient is non-recombinant human serum albumin:

In some embodiments, the non-recombinant human serum albumin is present at about 1% (w/v). In some embodiments, the non-recombinant human serum albumin is present at about 0.5% (w/v).

In some embodiments, the lentiviral vector is a VSV-G pseudotyped lentivirus

In some embodiments, the VSV-G pseudotyped lentivirus is present at a concentration of about 1.0E⁺⁰⁵ to about 1.0E⁺⁰⁹ TUs/mL.

In some embodiments, the VSV-G pseudotyped lentivirus comprises a VSV-G polypeptide comprising a mutation that corresponds to a mutation at position 182 of SEQ ID NO: 2.

In some embodiments, a pharmaceutical composition is provided, the pharmaceutical composition comprising a VSV-G pseudotyped lentivirus; about 10 mM to about 30 mM of a L-Histidine buffer; about 100 mM to about 200 mM of sodium chloride; about 1% (w/v) to about 5% (w/v) of sucrose; and about 0.1% (w/v) to about 2% (w/v) of a non-recombinant human serum albumin, wherein the pharmaceutical composition has a pH of about 5.5 to about 8.0. In some embodiments, the plant recombinant human serum albumin is Exbumin®.

In some embodiments, the VSV-G psuedotyped lentivirus is present at a concentration of about 1.0E⁺⁰⁵ to about 1.0E⁺⁰⁹ TUs/mL.

In some embodiments, the L-Histidine is present at a concentration of about 20 mM and the pharmaceutical composition has a pH of about 7.0.

In some embodiments, sodium chloride is present at a concentration of about 150 mM.

In some embodiments, sucrose is present at about 4% (w/v).

In some embodiments, the non-recombinant human serum albumin is present at about 0.5% (w/v). In some embodiments, the non-recombinant human serum albumin is present at about 1% (w/v).

In some embodiments, the VSV-G pseudotyped lentivirus comprises a VSV-G polypeptide comprising a mutation that corresponds to a mutation at position 182 of SEQ ID NO: 2.

In some embodiments, the VSV-G polypeptide comprises a mutation that corresponds to I182A, I182D or I182E as compared to a sequence of SEQ ID NO: 2.

In some embodiments, the polypeptide comprises a T214N and/or T352A mutation.

In some embodiments, the polypeptide comprises substitutions at positions I182, T214, and T352 of SEQ ID NO: 2.

In some embodiments, a pharmaceutical composition is provided, the pharmaceutical composition comprising a VSV-G polypeptide comprising a mutation that corresponds to a mutation at position 182, 214, and/or 352 of SEQ ID NO: 2; L-Histidine buffer at a concentration of about 10 mM to about 30 mM; sodium chloride at a concentration of about 100 mM to about 200 mM; sucrose in an amount of about 1% (w/v) to about 5% (w/v); and non-recombinant human serum albumin in an amount of about 0.1% (w/v) to about 2% (w/v), wherein the pharmaceutical composition has a pH of about 6.5 to about 7.5.

### BRIEF DESCRIPTION OF THE DRAWINGS

FIG. 1 illustrates the lentiviral vector process for ex vivo and in vivo use.

FIG. 2 illustrates the VSVG-LV formulation screening.

FIG. 3 illustrates the formulation development of intravenous and intralymphatic-compatible administration by stabilizing LV through two freeze-thaw cycles.

FIG. 4 illustrates the formulation confirmation study depicting the selection of histidine buffer.

FIGS. 5A and 5B illustrate optimal [NaCl] helping to minimize LV particle aggregation without impacting LV stability. FIG. 5A illustrates the effect of [NaCl] on particle size distribution. FIG. 5B illustrates the impact of [NaCl] on viral titer with fresh and 1×F/T samples.

FIGS. 6A, 6B, 6C, and 6D illustrate the results of a HSS formulation optimization study. FIG. 6A illustrates better formulation stability with 1% Exbumin. FIG. 6B illustrates the effect of [NaCl] on particle size distribution in the absence of Exbumin. FIG. 6C illustrates the effect of [NaCl] on particle size distribution in the presence of 1% Exbumin. FIG. 6D illustrates the effect of pH on particle size distribution for a 150 mM NaCl, 1% Exbumin formulation.

FIGS. 7A and 7B illustrate the comparison of stability of formulations containing recombinant HSA (Exbumin®) and non-recombinant HSA. FIG. 7A illustrates the results of a 3× freeze thaw cycle and incubation for 20 hours at various temperatures. FIG. 7B illustrates the results of a 3× freeze thaw cycle and incubation for 72 hours at various temperatures.

FIGS. 8A and 8B illustrate crystal structures of VSV-G bound to LDL-R. FIG. 8A illustrates the crystal structure of VSV-G bound to CR3 of the LDL-R. FIG. 8B illustrates the crystal structure of VSV-G bound to CR2 of the LDL-R.

FIGS. 9A and 9B illustrate the effect of adding negatively charged amino acids to the VSV-G:LDL-R binding interface on native tropism and fusogenicity. FIG. 9A illustrates the titration of VSV-G constructs on SupT1 cells. FIG. 9B illustrates functional titer of each construct calculated from the titration in FIG. 9A.

FIGS. 10A, 10B, 10C, 10D, and 10E illustrate an alignment of the ectodomains of different VSV-G proteins from different strains. Each of 10A, 10B, 10C, 10D, and 10E are portions from the same alignment, broken up into separate figures for ease of visualization.

### DETAILED DESCRIPTION

It is to be understood that the embodiments described herein are not limited to particular formulations, compositions and experimental conditions disclosed, as such formulations, compositions, and experimental conditions may vary. It is also to be understood that the terminology used herein is only for the purpose of describing particular embodiments, and it is not intended to be limiting.

Furthermore, the formulations, compositions, and experimental conditions described herein, unless otherwise indicated, use conventional molecular and cellular biological and immunological techniques known within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art.

Unless otherwise required by context, singular terms shall include pluralities, and plural terms shall include the singular.

That the disclosure may be more readily understood, select terms are defined below.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, where a phrase recites “about x to y,” the term “about” modifies both x and y and can be used interchangeably with the phrase “about x to about y” unless context dictates differently.

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the phrase “viral vector recovery” is defined as the amount of viral vector post-filtration compared to the amount of viral vector pre-filtration expressed as a percentage.

As used herein, the phrase “purified viral vector” is defined as viral vector which has been purified from cell culture harvest using at least one chromatography step.

As used herein, “non-concentrated” viral vector is a viral vector in a solution that has not undergone a concentration step during its production. As used herein, a “non-concentrated”viral vector does not have a limit on the concentration of viral particles therein, but rather, unless explicitly state otherwise, solely denotes whether a solution comprising a viral vector has undergone a concentration step or not.

As used herein, the term “contacting” means bringing together of two elements in an in vitro system or an in vivo system. For example, “contacting” virus or vector described herein with an individual or patient or cell includes the administration of the virus to an individual or patient, such as a human, as well as, for example, introducing a compound into a sample containing a cellular or purified preparation containing the cell.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as the glycine/serine sequences described herein link the two domains together.

A “disease” in an animal is a state of health wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, or 90%, 95% or even 99% identical at the amino acid level or nucleic acid to the sequence used for comparison. Other percentages of identity in reference to specific sequences are described herein.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

To the extent embodiments provided for herein, include compositions comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. In addition to these percentages, other percentages of identity are provided for herein. Identity between polypeptides can be determined by the Smith-Waterman homology search algorithm as implemented in the MPSRCH program (Oxford Molecular), using an affine gap search with parameters gap open penalty−12 and gap extension penalty=1. These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family that is able to infect non-dividing cells. Non-limiting examples of lentiviruses are HIV, SIV, and FIV. Vectors or viral-like particles derived from lentiviruses can be used to transduce cells and deliver genes or other molecules and have them expressed in a cell either in vitro, ex-vivo, or in vivo.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

“Parenteral” administration of a composition includes, e.g., subcutaneous (s.c.), intravenous (i.v.), intramuscular (i.m.), or intracisternal injection, intrathecal, or infusion techniques.

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pseudotyped” or “pseudotyped viral particle”, as used herein, refers to a viral particle bearing glycoproteins derived from other viruses having envelopes or a viral vector encoding envelope glycoproteins from a virus that is different from the parental virus. The host range of the vector particles can thus be expanded or altered depending on the type of cell surface receptor used by the glycoprotein. For example, a virus can be pseudotyped with a VSV-G mutant protein as provided for herein.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In some embodiments, the targeting moieties described herein that can be used to target the viral particles comprising the mutant VSV-G protein, or other viral structural proteins used to pseudotype a virus, can specifically bind to their target.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

As used herein, the terms “viral vector”, “virus”, and “viral particle” are understood to be synonymous, unless the context of the embodiment specifically or explicitly dictates otherwise. This understanding is true for specific viral vector, too. For examples, as used herein, the terms “lentiviral vector”, “lentivirus”, and “lentiviral particle” are understood to be synonymous, unless the context of the embodiment specifically or explicitly dictates otherwise.

A “carrier” or “delivery vehicle” includes viral particles, viruses, polylysine compounds, and liposomes, which facilitate transfer of nucleic acid into cells. A carrier or delivery vehicle can also be used to deliver a protein or peptide to a cell.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

### Pharmaceutical Compositions

The term “composition” as used herein means a product which results from the mixing or combining of more than one element or ingredient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer.

The phrase “pharmaceutically acceptable” is employed herein to refer to those agents of interest/compounds, salts, compositions, pharmaceutical dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some embodiments, pharmaceutically acceptable means approved by a regulatory agency of the federal or a state government, or listed in the U.S. Pharmacopeia or other generally recognized pharmacopeia for use in animals (e.g., mammals), and more particularly, in humans.

As used herein, “stable”, a “stable composition” or “stable pharmaceutical composition” refers to a composition or pharmaceutical composition that maintains the characteristics of the composition within a defined margin when subject to various stressors such as, but not limited to, heat, light, temperature, humidity, repeated freeze thaw cycles, and extended storage. Such characteristics include, but are not limited to, pH, presence of aggregates, functional titer, or concentration of protein excipient. In some embodiments, the defined margins for pH, presence of aggregates, functional titer, or concentration of protein excipient are as provided for herein.

The term “excipient” refers to a pharmacologically inactive substance formulated with an antibody, antigen-binding fragment thereof, viral vector, or any other pharmacologically active molecules as provided for herein.

In some embodiments, a pharmaceutical composition is provided, comprising a lentiviral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, the pharmaceutical composition comprises a lentiviral vector at a concentration of about 1.0E⁺⁰⁵ to about 1.0E⁺⁰⁹ TUs/mL.

In some embodiments, the lentiviral vector is present at a concentration of about 1.0E⁺⁰⁵ to about 1.0E⁺⁰⁹ TUs/mL, about 1.0E⁺⁰⁵ to about 1.0E⁺⁰⁸ TUs/mL, about 1.0E⁺⁰⁵ to about 1.0E⁺⁰⁷ TUs/mL, about 1.0E⁺⁰⁵ to about 1.0E⁺⁰⁶ TUs/mL, about 1.0E⁺⁰⁶ to about 1.0E⁺⁰⁹ TUs/mL, about 1.0E⁺⁰⁶ to about 1.0E⁺⁰⁸ TUs/mL, about 1.0E⁺⁰⁶ to about 1.0E⁺⁰⁷ TUs/mL, about 1.0E⁺⁰⁷ to about 1.0E⁺⁰⁹ TUs/mL, about 1.0E⁺⁰⁷ to about 1.0E⁺⁰⁸ TUs/mL, or about 1.0E⁺⁰⁸ to about 1.0E⁺⁰⁹ TUs/mL.

In some embodiments, the lentiviral vector is present at a concentration of about 1.0E⁺⁰⁵ to about 1.0E⁺⁰⁹ TUs/mL. In some embodiments, the lentiviral vector is present at a concentration of about 1.0E⁺⁰⁵ to about 1.0E⁺⁰⁸ TUs/mL. In some embodiments, the lentiviral vector is present at a concentration of about 1.0E⁺⁰⁵ to about 1.0E⁺⁰⁷ TUs/mL. In some embodiments, the lentiviral vector is present at a concentration of about 1.0E⁺⁰⁵ to about 1.0E⁺⁰⁶ TUs/mL. In some embodiments, the lentiviral vector is present at a concentration of about 1.0E⁺⁰% to about 1.0E⁺⁰⁹ TUs/mL. In some embodiments, the lentiviral vector is present at a concentration of about 1.0E⁺⁰% to about 1.0E⁺⁰⁸ TUs/mL. In some embodiments, the lentiviral vector is present at a concentration of about 1.0E⁺⁰⁶ to about 1.0E⁺⁰⁷ TUs/mL. In some embodiments, the lentiviral vector is present at a concentration of about 1.0E⁺⁰⁷ to about 1.0E⁺⁰⁹ TUs/mL. In some embodiments, the lentiviral vector is present at a concentration of about 1.0E⁺⁰⁷ to about 1.0E⁺⁰⁸ TUs/mL. In some embodiments, the lentiviral vector is present at a concentration of about 1.0E⁺⁰⁸ to about 1.0E⁺⁰⁹ TUs/mL.

In some embodiments, the lentiviral vector is present at a concentration of at least 1.0E⁺⁰⁵. In some embodiments, the lentiviral vector is present at a concentration of at least 2.0E⁺⁰⁵. In some embodiments, the lentiviral vector is present at a concentration of at least 3.0E⁺⁰⁵ In some embodiments, the lentiviral vector is present at a concentration of at least 4.0E⁺⁰⁵. In some embodiments, the lentiviral vector is present at a concentration of at least 5.0E⁺⁰⁵. In some embodiments, the lentiviral vector is present at a concentration of at least 6.0E⁺⁰⁵. In some embodiments, the lentiviral vector is present at a concentration of at least 7.0E⁺⁰⁵. In some embodiments, the lentiviral vector is present at a concentration of at least 8.0E⁺⁰⁵. In some embodiments, the lentiviral vector is present at a concentration of at least 9.0E⁺⁰⁵. In some embodiments, the lentiviral vector is present at a concentration of at least 1.0E⁺⁰⁶. In some embodiments, the lentiviral vector is present at a concentration of at least 2.0E⁺⁰⁶. In some embodiments, the lentiviral vector is present at a concentration of at least 3.0E⁺⁰⁶. In some embodiments, the lentiviral vector is present at a concentration of at least 4.0E⁺⁰⁶. In some embodiments, the lentiviral vector is present at a concentration of at least 5.0E⁺⁰⁶. In some embodiments, the lentiviral vector is present at a concentration of at least 6.0E⁺⁰⁶. In some embodiments, the lentiviral vector is present at a concentration of at least 7.0E⁺⁰⁶. In some embodiments, the lentiviral vector is present at a concentration of at least 8.0E⁺⁰⁶. In some embodiments, the lentiviral vector is present at a concentration of at least 9.0E⁺⁰⁶. In some embodiments, the lentiviral vector is present at a concentration of at least 1.0E⁺⁰⁷. In some embodiments, the lentiviral vector is present at a concentration of at least 2.0E⁺⁰⁷. In some embodiments, the lentiviral vector is present at a concentration of at least 3.0E⁺⁰⁷. In some embodiments, the lentiviral vector is present at a concentration of at least 4.0E⁺⁰⁷ In some embodiments, the lentiviral vector is present at a concentration of at least 5.0E⁺⁰⁷. In some embodiments, the lentiviral vector is present at a concentration of at least 6.0E⁺⁰⁷. In some embodiments, the lentiviral vector is present at a concentration of at least 7.0E⁺⁰⁷. In some embodiments, the lentiviral vector is present at a concentration of at least 8.0E⁺⁰⁷. In some embodiments, the lentiviral vector is present at a concentration of at least 9.0E⁺⁰⁷. In some embodiments, the lentiviral vector is present at a concentration of at least 1.0E⁺⁰⁸. In some embodiments, the lentiviral vector is present at a concentration of at least 2.0E⁺⁰⁸. In some embodiments, the lentiviral vector is present at a concentration of at least 3.0E⁺⁰⁸ In some embodiments, the lentiviral vector is present at a concentration of at least 4.0E⁺⁰⁸. In some embodiments, the lentiviral vector is present at a concentration of at least 5.0E⁺⁰⁸. In some embodiments, the lentiviral vector is present at a concentration of at least 6.0E⁺⁰⁸. In some embodiments, the lentiviral vector is present at a concentration of at least 7.0E⁺⁰⁸. In some embodiments, the lentiviral vector is present at a concentration of at least 8.0E⁺⁰⁸. In some embodiments, the lentiviral vector is present at a concentration of at least 9.0E⁺⁰⁸. In some embodiments, the lentiviral vector is present at a concentration of at least 1.0E^{+09.}

In some embodiments, the pharmaceutically acceptable buffer has a concentration of about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 25 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 25 mM, about 15 mM to about 100 mM, about 15 mM to about 90 mM, about 15 mM to about 80 mM, about 15 mM to about 70 mM, about 15 mM to about 60 mM, about 15 mM to about 50 mM, about 15 mM to about 40 mM, about 15 mM to about 30 mM, or about 15 mM to about 25 mM.

In some embodiments, the pharmaceutically acceptable buffer has a concentration of about 5 mM to about 100 mM, about 5 mM to about 70 mM, about 5 mM to about 60 mM, about 10 mM to about 60 mM, about 10 mM to about 50 mM, about 10 mM to about 40 mM, about 15 mM to about 30 mM, or about 15 mM to about 25 mM.

In some embodiments, the pharmaceutically acceptable buffer has a concentration of about 5 mM. In some embodiments, the pharmaceutically acceptable buffer has a concentration of about 10 mM. In some embodiments, the pharmaceutically acceptable buffer has a concentration of about 15 mM. In some embodiments, the pharmaceutically acceptable buffer has a concentration of about 20 mM. In some embodiments, the pharmaceutically acceptable buffer has a concentration of about 25 mM. In some embodiments, the pharmaceutically acceptable buffer has a concentration of about 30 mM. In some embodiments, the pharmaceutically acceptable buffer has a concentration of about 35 mM. In some embodiments, the pharmaceutically acceptable buffer has a concentration of about 40 mM. In some embodiments, the pharmaceutically acceptable buffer has a concentration of about 45 mM. In some embodiments, the pharmaceutically acceptable buffer has a concentration of about 50 mM. In some embodiments, the pharmaceutically acceptable buffer has a concentration of about 55 mM. In some embodiments, the pharmaceutically acceptable buffer has a concentration of about 60 mM. In some embodiments, the pharmaceutically acceptable buffer has a concentration of about 65 mM. In some embodiments, the pharmaceutically acceptable buffer has a concentration of about 70 mM. In some embodiments, the pharmaceutically acceptable buffer has a concentration of about 75 mM. In some embodiments, the pharmaceutically acceptable buffer has a concentration of about 80 mM. In some embodiments, the pharmaceutically acceptable buffer has a concentration of about 85 mM. In some embodiments, the pharmaceutically acceptable buffer has a concentration of about 90 mM. In some embodiments, the pharmaceutically acceptable buffer has a concentration of about 95 mM. In some embodiments, the pharmaceutically acceptable buffer has a concentration of about 100 mM.

In some embodiments, the pharmaceutically acceptable buffer is selected from histidine buffer, HEPES, phosphoric acid buffer, citric acid buffer, acetic acid buffer, succinic acid buffer, phosphate buffer, acetate buffer, citrate buffer, succinate buffer, ascorbic acid buffer, glutamic acid buffer, lactic acid buffer, maleic acid buffer, trometamol buffer, gluconic acid buffer, or combinations thereof.

In some embodiments, the pharmaceutically acceptable buffer is histidine buffer. In some embodiments, the pharmaceutically acceptable buffer is HEPES. In some embodiments, the pharmaceutically acceptable buffer is phosphoric acid buffer. In some embodiments, the pharmaceutically acceptable buffer is citric acid buffer. In some embodiments, the pharmaceutically acceptable buffer is acetic acid buffer. In some embodiments, the pharmaceutically acceptable buffer is succinic acid buffer. In some embodiments, the pharmaceutically acceptable buffer is phosphate buffer. In some embodiments, the pharmaceutically acceptable buffer is acetate buffer. In some embodiments, the pharmaceutically acceptable buffer is citrate buffer. In some embodiments, the pharmaceutically acceptable buffer is succinate buffer. In some embodiments, the pharmaceutically acceptable buffer is ascorbic acid buffer. In some embodiments, the pharmaceutically acceptable buffer is glutamic acid buffer. In some embodiments, the pharmaceutically acceptable buffer is lactic acid buffer. In some embodiments, the pharmaceutically acceptable buffer is maleic acid buffer. In some embodiments, the pharmaceutically acceptable buffer is trometamol buffer. In some embodiments, the pharmaceutically acceptable buffer is gluconic acid buffer.

In some embodiments, the pharmaceutically acceptable buffer is a histidine buffer.

In some embodiments, the histidine buffer has a concentration of about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 25 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 25 mM, about 15 mM to about 100 mM, about 15 mM to about 90 mM, about 15 mM to about 80 mM, about 15 mM to about 70 mM, about 15 mM to about 60 mM, about 15 mM to about 50 mM, about 15 mM to about 40 mM, about 15 mM to about 30 mM, or about 15 mM to about 25 mM.

In some embodiments, the histidine buffer has a concentration of about 5 mM to about 100 mM, about 5 mM to about 70 mM, about 5 mM to about 60 mM, about 10 mM to about 60 mM, about 10 mM to about 50 mM, about 10 mM to about 40 mM, about 15 mM to about 30 mM, or about 15 mM to about 25 mM.

In some embodiments, the histidine buffer has a concentration of about 5 mM. In some embodiments, the histidine buffer has a concentration of about 10 mM. In some embodiments, the histidine buffer has a concentration of about 11 mM. In some embodiments, the histidine buffer has a concentration of about 12 mM. In some embodiments, the histidine buffer has a concentration of about 13 mM. In some embodiments, the histidine buffer has a concentration of about 14 mM. In some embodiments, the histidine buffer has a concentration of about 15 mM. In some embodiments, the histidine buffer has a concentration of about 16 mM. In some embodiments, the histidine buffer has a concentration of about 17 mM. In some embodiments, the histidine buffer has a concentration of about 18 mM. In some embodiments, the histidine buffer has a concentration of about 19 mM. In some embodiments, the histidine buffer has a concentration of about 20 mM. In some embodiments, the histidine buffer has a concentration of about 25 mM. In some embodiments, the histidine buffer has a concentration of about 30 mM. In some embodiments, the histidine buffer has a concentration of about 35 mM. In some embodiments, the histidine buffer has a concentration of about 40 mM. In some embodiments, the histidine buffer has a concentration of about 45 mM. In some embodiments, the histidine buffer has a concentration of about 50 mM. In some embodiments, histidine buffer has a concentration of about 55 mM. In some embodiments, the histidine buffer has a concentration of about 60 mM. In some embodiments, the histidine buffer has a concentration of about 65 mM. In some embodiments, the histidine buffer has a concentration of about 70 mM. In some embodiments, the histidine buffer has a concentration of about 75 mM. In some embodiments, the histidine buffer has a concentration of about 80 mM. In some embodiments, the histidine buffer has a concentration of about 85 mM. In some embodiments, the histidine buffer has a concentration of about 90 mM. In some embodiments, the histidine buffer has a concentration of about 95 mM. In some embodiments, the histidine buffer has a concentration of about 100 mM.

In some embodiments, the histidine buffer has a concentration of about 14 mM. In some embodiments, the histidine buffer is present at a concentration of about 20 mM.

In some embodiments, the pharmaceutical composition has a pH of about 6 to about 8, about 6.5 to about 8, about 7 to about 8, about 7.5 to about 8, about 6 to about 7.5, about 6 to about 7, about 6 to about 6.5, about 6.5 to about 7.5, or any value or range therein. In some embodiments, the pharmaceutical composition has a pH of about 6.5 to about 7.5.

In some embodiments, the pharmaceutical composition has a pH of about 6.0. In some embodiments, the pharmaceutical composition has a pH of about 6.1. In some embodiments, the pharmaceutical composition has a pH of about 6.2. In some embodiments, the pharmaceutical composition has a pH of about 6.3. In some embodiments, the pharmaceutical composition has a pH of about 6.4. In some embodiments, the pharmaceutical composition has a pH of about 6.5. In some embodiments, the pharmaceutical composition has a pH of about 6.6. In some embodiments, the pharmaceutical composition has a pH of about 6.7. In some embodiments, the pharmaceutical composition has a pH of about 6.8. In some embodiments, the pharmaceutical composition has a pH of about 6.9. In some embodiments, the pharmaceutical composition has a pH of about 7.0. In some embodiments, the pharmaceutical composition has a pH of about 7.1. In some embodiments, the pharmaceutical composition has a pH of about 7.2. In some embodiments, the pharmaceutical composition has a pH of about 7.3. In some embodiments, the pharmaceutical composition has a pH of about 7.4. In some embodiments, the pharmaceutical composition has a pH of about 7.5. In some embodiments, the pharmaceutical composition has a pH of about 7.6. In some embodiments, the pharmaceutical composition has a pH of about 7.7. In some embodiments, the pharmaceutical composition has a pH of about 7.8. In some embodiments, the pharmaceutical composition has a pH of about 7.9. In some embodiments, the pharmaceutical composition has a pH of about 8.0.

In some embodiments, the pharmaceutical composition has a pH of about 7.0.

In some embodiments, the pharmaceutically acceptable buffer is a histidine buffer and the pharmaceutical composition has a pH of about 6 to about 8. In some embodiments, the pharmaceutically acceptable buffer is a histidine buffer and the pharmaceutical composition has a pH of about 6.5 to about 7.5.

In some embodiments, the pharmaceutically acceptable buffer is a histidine buffer and the pharmaceutical composition has a pH of about 6.0. In some embodiments, the pharmaceutically acceptable buffer is a histidine buffer and the pharmaceutical composition has a pH of about 6.1. In some embodiments, the pharmaceutically acceptable buffer is a histidine buffer and the pharmaceutical composition has a pH of about 6.2. In some embodiments, the pharmaceutically acceptable buffer is a histidine buffer and the pharmaceutical composition has a pH of about 6.3. In some embodiments, the pharmaceutically acceptable buffer is a histidine buffer and the pharmaceutical composition has a pH of about 6.4. In some embodiments, the pharmaceutically acceptable buffer is a histidine buffer and the pharmaceutical composition has a pH of about 6.5. In some embodiments, the pharmaceutically acceptable buffer is a histidine buffer and the pharmaceutical composition has a pH of about 6.6. In some embodiments, the pH pharmaceutically acceptable buffer is a histidine buffer and the pharmaceutical composition has a pH of about 6.7. In some embodiments, the pharmaceutically acceptable buffer is a histidine buffer and the pharmaceutical composition has a pH of about 6.8. In some embodiments, the pharmaceutically acceptable buffer is a histidine buffer and the pharmaceutical composition has a pH of about 6.9. In some embodiments, the pharmaceutically acceptable buffer is a histidine buffer and the pharmaceutical composition has a pH of about 7.0. In some embodiments, the pharmaceutically acceptable buffer is a histidine buffer and the pharmaceutical composition has a pH of about 7.1. In some embodiments, the pharmaceutically acceptable buffer is a histidine buffer and the pharmaceutical composition has a pH of about 7.2. In some embodiments, the pharmaceutically acceptable buffer is a histidine buffer and the pharmaceutical composition has a pH of about 7.3. In some embodiments, the pharmaceutically acceptable buffer is a histidine buffer and the pharmaceutical composition has a pH of about 7.4. In some embodiments, the pharmaceutically acceptable buffer is a histidine buffer and the pharmaceutical composition has a pH of 7.5. In some embodiments, the pharmaceutically acceptable buffer is a histidine buffer and the pharmaceutical composition has a pH of about 7.6. In some embodiments, the pharmaceutically acceptable buffer is a histidine buffer and the pharmaceutical composition has a pH of about 7.7. In some embodiments, the pharmaceutically acceptable buffer is a histidine buffer and the pharmaceutical composition has a pH of about 7.8. In some embodiments, the pharmaceutically acceptable buffer is a histidine buffer and the pharmaceutical composition has a pH of about 7.9. In some embodiments, the pharmaceutically acceptable buffer is a histidine buffer and the pharmaceutical composition has a pH of about 8.0.

In some embodiments, the pharmaceutically acceptable buffer is a histidine buffer and the pharmaceutical composition has a pH of about 7.0.

In some embodiments, the pharmaceutically acceptable salt is present at a concentration of about 100 mM to about 800 mM, or any value or range in-between.

In some embodiments, the pharmaceutically acceptable salt is present at a concentration of about 100 mM to about 800 mM. In some embodiments, the pharmaceutically acceptable salt is present at a concentration of about 100 mM to about 700 mM. In some embodiments, the pharmaceutically acceptable salt is present at a concentration of about 100 mM to about 600 mM. In some embodiments, the pharmaceutically acceptable salt is present at a concentration of about 100 mM to about 500 mM. In some embodiments, the pharmaceutically acceptable salt is present at a concentration of about 100 mM to about 400 mM. In some embodiments, the pharmaceutically acceptable salt is present at a concentration of about 100 mM to about 300 mM. In some embodiments, the pharmaceutically acceptable salt is present at a concentration of about 100 mM to about 250 mM. In some embodiments, the pharmaceutically acceptable salt is present at a concentration of about 100 mM to about 200 mM. In some embodiments, the pharmaceutically acceptable salt is present at a concentration of about 100 mM to about 175 mM. In some embodiments, the pharmaceutically acceptable salt is present at a concentration of about 100 mM to about 150 mM. In some embodiments, the pharmaceutically acceptable salt is present at a concentration of about 100 mM to about 125 mM.

In some embodiments, the pharmaceutically acceptable salt is present at a concentration of about 100 mM to about 800 mM. In some embodiments, the pharmaceutically acceptable salt is present at a concentration of about 125 mM to about 800 mM. In some embodiments, the pharmaceutically acceptable salt is present at a concentration of about 150 mM to about 800 mM. In some embodiments, the pharmaceutically acceptable salt is present at a concentration of about 175 mM to about 800 mM. In some embodiments, the pharmaceutically acceptable salt is present at a concentration of about 200 mM to about 800 mM. In some embodiments, the pharmaceutically acceptable salt is present at a concentration of about 250 mM to about 800 mM. In some embodiments, the pharmaceutically acceptable salt is present at a concentration of about 300 mM to about 800 mM. In some embodiments, the pharmaceutically acceptable salt is present at a concentration of about 350 mM to about 800 mM. In some embodiments, the pharmaceutically acceptable salt is present at a concentration of about 400 mM to about 800 mM. In some embodiments, the pharmaceutically acceptable salt is present at a concentration of about 500 mM to about 800 mM. In some embodiments, the pharmaceutically acceptable salt is present at a concentration of about 600 mM to about 800 mM. In some embodiments, the pharmaceutically acceptable salt is present at a concentration of about 700 mM to about 800 mM.

In some embodiments, the pharmaceutically acceptable salt is present at a concentration of about 100 mM. In some embodiments, the pharmaceutically acceptable salt is present at a concentration of about 110 mM. In some embodiments, the pharmaceutically acceptable salt is present at a concentration of about 120 mM. In some embodiments, the pharmaceutically acceptable salt is present at a concentration of about 130 mM. In some embodiments, the pharmaceutically acceptable salt is present at a concentration of about 140 mM. In some embodiments, the pharmaceutically acceptable salt is present at a concentration of about 150 mM. In some embodiments, the pharmaceutically acceptable salt is present at a concentration of about 160 mM. In some embodiments, the pharmaceutically acceptable salt is present at a concentration of about 170 mM. In some embodiments, the pharmaceutically acceptable salt is present at a concentration of about 180 mM. In some embodiments, the pharmaceutically acceptable salt is present at a concentration of about 190 mM. In some embodiments, the pharmaceutically acceptable salt is present at a concentration of about 200 mM In some embodiments, the pharmaceutically acceptable salt is present at a concentration of about 210 mM. In some embodiments, the pharmaceutically acceptable salt is present at a concentration of about 220 mM. In some embodiments, the pharmaceutically acceptable salt is present at a concentration of about 230 mM. In some embodiments, the pharmaceutically acceptable salt is present at a concentration of about 240 mM. In some embodiments, the pharmaceutically acceptable salt is present at a concentration of about 250 mM. In some embodiments, the pharmaceutically acceptable salt is present at a concentration of about 260 mM. In some embodiments, the pharmaceutically acceptable salt is present at a concentration of about 270 mM. In some embodiments, the pharmaceutically acceptable salt is present at a concentration of about 280 mM. In some embodiments, the pharmaceutically acceptable salt is present at a concentration of about 290 mM. In some embodiments, the pharmaceutically acceptable salt is present at a concentration of about 300 mM. In some embodiments, the pharmaceutically acceptable salt is present at a concentration of about 310 mM. In some embodiments, the pharmaceutically acceptable salt is present at a concentration of about 320 mM. In some embodiments, the pharmaceutically acceptable salt is present at a concentration of about 330 mM. In some embodiments, the pharmaceutically acceptable salt is present at a concentration of about 340 mM. In some embodiments, the pharmaceutically acceptable salt is present at a concentration of about 350 mM. In some embodiments, the pharmaceutically acceptable salt is present at a concentration of about 360 mM. In some embodiments, the pharmaceutically acceptable salt is present at a concentration of about 370 mM. In some embodiments, the pharmaceutically acceptable salt is present at a concentration of about 380 mM. In some embodiments, the pharmaceutically acceptable salt is present at a concentration of about 390 mM. In some embodiments, the pharmaceutically acceptable salt is present at a concentration of about 400 mM. In some embodiments, the pharmaceutically acceptable salt is present at a concentration of about 410 mM. In some embodiments, the pharmaceutically acceptable salt is present at a concentration of about 420 mM. In some embodiments, the pharmaceutically acceptable salt is present at a concentration of about 430 mM. In some embodiments, the pharmaceutically acceptable salt is present at a concentration of about 440 mM. In some embodiments, the pharmaceutically acceptable salt is present at a concentration of about 450 mM. In some embodiments, the pharmaceutically acceptable salt is present at a concentration of about 460 mM. In some embodiments, the pharmaceutically acceptable salt is present at a concentration of about 470 mM. In some embodiments, the pharmaceutically acceptable salt is present at a concentration of about 480 mM. In some embodiments, the pharmaceutically acceptable salt is present at a concentration of about 490 mM. In some embodiments, the pharmaceutically acceptable salt is present at a concentration of about 500 mM. In some embodiments, the pharmaceutically acceptable salt is present at a concentration of about 510 mM. In some embodiments, the pharmaceutically acceptable salt is present at a concentration of about 520 mM. In some embodiments, the pharmaceutically acceptable salt is present at a concentration of about 530 mM. In some embodiments, the pharmaceutically acceptable salt is present at a concentration of about 540 mM. In some embodiments, the pharmaceutically acceptable salt is present at a concentration of about 550 mM. In some embodiments, the pharmaceutically acceptable salt is present at a concentration of about 560 mM. In some embodiments, the pharmaceutically acceptable salt is present at a concentration of about 570 mM. In some embodiments, the pharmaceutically acceptable salt is present at a concentration of about 580 mM. In some embodiments, the pharmaceutically acceptable salt is present at a concentration of about 590 mM. In some embodiments, the pharmaceutically acceptable salt is present at a concentration of about 600 mM. In some embodiments, the pharmaceutically acceptable salt is present at a concentration of about 610 mM. In some embodiments, the pharmaceutically acceptable salt is present at a concentration of about 620 mM. In some embodiments, the pharmaceutically acceptable salt is present at a concentration of about 630 mM. In some embodiments, the pharmaceutically acceptable salt is present at a concentration of about 640 mM. In some embodiments, the pharmaceutically acceptable salt is present at a concentration of about 650 mM. In some embodiments, the pharmaceutically acceptable salt is present at a concentration of about 660 mM. In some embodiments, the pharmaceutically acceptable salt is present at a concentration of about 670 mM. In some embodiments, the pharmaceutically acceptable salt is present at a concentration of about 680 mM. In some embodiments, the pharmaceutically acceptable salt is present at a concentration of about 690 mM. In some embodiments, the pharmaceutically acceptable salt is present at a concentration of about 700 mM. In some embodiments, the pharmaceutically acceptable salt is present at a concentration of about 710 mM. In some embodiments, the pharmaceutically acceptable salt is present at a concentration of about 720 mM. In some embodiments, the pharmaceutically acceptable salt is present at a concentration of about 730 mM. In some embodiments, the pharmaceutically acceptable salt is present at a concentration of about 740 mM. In some embodiments, the pharmaceutically acceptable salt is present at a concentration of about 750 mM. In some embodiments, the pharmaceutically acceptable salt is present at a concentration of about 760 mM. In some embodiments, the pharmaceutically acceptable salt is present at a concentration of about 770 mM. In some embodiments, the pharmaceutically acceptable salt is present at a concentration of about 780 mM. In some embodiments, the pharmaceutically acceptable salt is present at a concentration of about 790 mM. In some embodiments, the pharmaceutically acceptable salt is present at a concentration of about 800 mM.

In some embodiments, the pharmaceutically acceptable salt is any appropriate pharmaceutically acceptable salt. Pharmaceutically acceptable salts are known in the art, and any such salt is within the scope of the present disclosure. In some embodiments, the pharmaceutically acceptable salt is selected from sodium chloride, potassium chloride, magnesium chloride, calcium chloride, or combinations thereof.

In some embodiments, the pharmaceutically acceptable salt is sodium chloride. In some embodiments, the pharmaceutically acceptable salt is potassium chloride. In some embodiments, the pharmaceutically acceptable salt is magnesium chloride. In some embodiments, the pharmaceutically acceptable salt is calcium chloride.

In some embodiments, the pharmaceutically acceptable salt is sodium chloride.

In some embodiments, the sodium chloride is present at a concentration of about 100 mM to about 800 mM, or any value or range in-between. In some embodiments, the sodium chloride is present at a concentration of about 100 mM to about 700 mM. In some embodiments, the sodium chloride is present at a concentration of about 100 mM to about 600 mM. In some embodiments, the sodium chloride is present at a concentration of about 100 mM to about 500 mM. In some embodiments, the sodium chloride is present at a concentration of about 100 mM to about 400 mM. In some embodiments, the sodium chloride is present at a concentration of about 100 mM to about 300 mM. In some embodiments, the sodium chloride is present at a concentration of about 100 mM to about 250 mM. In some embodiments, the sodium chloride is present at a concentration of about 100 mM to about 200 mM. In some embodiments, the sodium chloride is present at a concentration of about 100 mM to about 175 mM. In some embodiments, the sodium chloride is present at a concentration of about 100 mM to about 150 mM. In some embodiments, the sodium chloride is present at a concentration of about 100 mM to about 125 mM.

In some embodiments, the sodium chloride is present at a concentration of about 100 mM to about 800 mM. In some embodiments, the sodium chloride is present at a concentration of about 125 mM to about 800 mM. In some embodiments, the sodium chloride is present at a concentration of about 150 mM to about 800 mM. In some embodiments, the sodium chloride is present at a concentration of about 175 mM to about 800 mM. In some embodiments, the sodium chloride is present at a concentration of about 200 mM to about 800 mM. In some embodiments, the sodium chloride is present at a concentration of about 250 mM to about 800 mM. In some embodiments, the sodium chloride is present at a concentration of about 300 mM to about 800 mM. In some embodiments, the sodium chloride is present at a concentration of about 350 mM to about 800 mM. In some embodiments, the sodium chloride is present at a concentration of about 400 mM to about 800 mM. In some embodiments, the sodium chloride is present at a concentration of about 500 mM to about 800 mM. In some embodiments, the sodium chloride is present at a concentration of about 600 mM to about 800 mM. In some embodiments, the sodium chloride is present at a concentration of about 700 mM to about 800 mM.

In some embodiments, the sodium chloride is present at a concentration of about 100 mM. In some embodiments, the sodium chloride is present at a concentration of about 110 mM. In some embodiments, the sodium chloride is present at a concentration of about 120 mM. In some embodiments, the sodium chloride is present at a concentration of about 130 mM. In some embodiments, the sodium chloride is present at a concentration of about 140 mM. In some embodiments, the sodium chloride is present at a concentration of about 150 mM. In some embodiments, the sodium chloride is present at a concentration of about 160 mM. In some embodiments, the sodium chloride is present at a concentration of about 170 mM. In some embodiments, the sodium chloride is present at a concentration of about 180 mM. In some embodiments, the sodium chloride is present at a concentration of about 190 mM. In some embodiments, the sodium chloride is present at a concentration of about 200 mM. In some embodiments, the sodium chloride is present at a concentration of about 210 mM. In some embodiments, the sodium chloride is present at a concentration of about 220 mM. In some embodiments, the sodium chloride is present at a concentration of about 230 mM. In some embodiments, the sodium chloride is present at a concentration of about 240 mM. In some embodiments, the sodium chloride is present at a concentration of about 250 mM. In some embodiments, the sodium chloride is present at a concentration of about 260 mM. In some embodiments, the sodium chloride is present at a concentration of about 270 mM. In some embodiments, the sodium chloride is present at a concentration of about 280 mM. In some embodiments, the sodium chloride is present at a concentration of about 290 mM. In some embodiments, the sodium chloride is present at a concentration of about 300 mM. In some embodiments, the sodium chloride is present at a concentration of about 310 mM. In some embodiments, the sodium chloride is present at a concentration of about 320 mM. In some embodiments, the sodium chloride is present at a concentration of about 330 mM. In some embodiments, the sodium chloride is present at a concentration of about 340 mM. In some embodiments, the sodium chloride is present at a concentration of about 350 mM. In some embodiments, the sodium chloride is present at a concentration of about 360 mM. In some embodiments, the sodium chloride is present at a concentration of about 370 mM. In some embodiments, the sodium chloride is present at a concentration of about 380 mM. In some embodiments, the sodium chloride is present at a concentration of about 390 mM. In some embodiments, the sodium chloride is present at a concentration of about 400 mM. In some embodiments, the sodium chloride is present at a concentration of about 410 mM. In some embodiments, the sodium chloride is present at a concentration of about 420 mM. In some embodiments, the sodium chloride is present at a concentration of about 430 mM. In some embodiments, the sodium chloride is present at a concentration of about 440 mM. In some embodiments, the sodium chloride is present at a concentration of about 450 mM. In some embodiments, the sodium chloride is present at a concentration of about 460 mM. In some embodiments, the sodium chloride is present at a concentration of about 470 mM. In some embodiments, the sodium chloride is present at a concentration of about 480 mM. In some embodiments, the sodium chloride is present at a concentration of about 490 mM. In some embodiments, the sodium chloride is present at a concentration of about 500 mM. In some embodiments, the sodium chloride is present at a concentration of about 510 mM. In some embodiments, the sodium chloride is present at a concentration of about 520 mM. In some embodiments, the sodium chloride is present at a concentration of about 530 mM. In some embodiments, the sodium chloride is present at a concentration of about 540 mM. In some embodiments, the sodium chloride is present at a concentration of about 550 mM. In some embodiments, the sodium chloride is present at a concentration of about 560 mM. In some embodiments, the sodium chloride is present at a concentration of about 570 mM. In some embodiments, the sodium chloride is present at a concentration of about 580 mM. In some embodiments, the sodium chloride is present at a concentration of about 590 mM. In some embodiments, the sodium chloride is present at a concentration of about 600 mM. In some embodiments, the sodium chloride is present at a concentration of about 610 mM. In some embodiments, the sodium chloride is present at a concentration of about 620 mM. In some embodiments, the sodium chloride is present at a concentration of about 630 mM. In some embodiments, the sodium chloride is present at a concentration of about 640 mM. In some embodiments, the sodium chloride is present at a concentration of about 650 mM. In some embodiments, the sodium chloride is present at a concentration of about 660 mM. In some embodiments, the sodium chloride is present at a concentration of about 670 mM. In some embodiments, the sodium chloride is present at a concentration of about 680 mM. In some embodiments, the sodium chloride is present at a concentration of about 690 mM. In some embodiments, the sodium chloride is present at a concentration of about 700 mM. In some embodiments, the sodium chloride is present at a concentration of about 710 mM. In some embodiments, the sodium chloride is present at a concentration of about 720 mM. In some embodiments, the sodium chloride is present at a concentration of about 730 mM. In some embodiments, the sodium chloride is present at a concentration of about 740 mM. In some embodiments, the sodium chloride is present at a concentration of about 750 mM. In some embodiments, the sodium chloride is present at a concentration of about 760 mM. In some embodiments, the sodium chloride is present at a concentration of about 770 mM. In some embodiments, the sodium chloride is present at a concentration of about 780 mM. In some embodiments, the sodium chloride is present at a concentration of about 790 mM. In some embodiments, the sodium chloride is present at a concentration of about 800 mM.

In some embodiments, the sodium chloride is present at a concentration of about 150 mM.

In some embodiments, the pharmaceutically acceptable salt is potassium chloride.

In some embodiments, the potassium chloride is present at a concentration of about 100 mM to about 800 mM, or any value or range in-between. In some embodiments, the potassium chloride is present at a concentration of about 100 mM to about 700 mM. In some embodiments, the potassium chloride is present at a concentration of about 100 mM to about 600 mM. In some embodiments, the potassium chloride is present at a concentration of about 100 mM to about 500 mM. In some embodiments, the potassium chloride is present at a concentration of about 100 mM to about 400 mM. In some embodiments, the potassium chloride is present at a concentration of about 100 mM to about 300 mM. In some embodiments, the potassium chloride is present at a concentration of about 100 mM to about 250 mM. In some embodiments, the potassium chloride is present at a concentration of about 100 mM to about 200 mM. In some embodiments, the potassium chloride is present at a concentration of about 100 mM to about 175 mM. In some embodiments, the potassium chloride is present at a concentration of about 100 mM to about 150 mM. In some embodiments, the potassium chloride is present at a concentration of about 100 mM to about 125 mM.

In some embodiments, the potassium chloride is present at a concentration of about 100 mM to about 800 mM. In some embodiments, the potassium chloride is present at a concentration of about 125 mM to about 800 mM. In some embodiments, the potassium chloride is present at a concentration of about 150 mM to about 800 mM. In some embodiments, the potassium chloride is present at a concentration of about 175 mM to about 800 mM. In some embodiments, the potassium chloride is present at a concentration of about 200 mM to about 800 mM. In some embodiments, the potassium chloride is present at a concentration of about 250 mM to about 800 mM. In some embodiments, the potassium chloride is present at a concentration of about 300 mM to about 800 mM. In some embodiments, the potassium chloride is present at a concentration of about 350 mM to about 800 mM. In some embodiments, the potassium chloride is present at a concentration of about 400 mM to about 800 mM. In some embodiments, the potassium chloride is present at a concentration of about 500 mM to about 800 mM. In some embodiments, the potassium chloride is present at a concentration of about 600 mM to about 800 mM. In some embodiments, the potassium chloride is present at a concentration of about 700 mM to about 800 mM.

In some embodiments, the potassium chloride is present at a concentration of about 100 mM. In some embodiments, the potassium chloride is present at a concentration of about 110 mM. In some embodiments, the potassium chloride is present at a concentration of about 120 mM. In some embodiments, the potassium chloride is present at a concentration of about 130 mM. In some embodiments, the potassium chloride is present at a concentration of about 140 mM. In some embodiments, the potassium chloride is present at a concentration of about 150 mM. In some embodiments, the potassium chloride is present at a concentration of about 160 mM. In some embodiments, the potassium chloride is present at a concentration of about 170 mM. In some embodiments, the potassium chloride is present at a concentration of about 180 mM. In some embodiments, the potassium chloride is present at a concentration of about 190 mM. In some embodiments, the potassium chloride is present at a concentration of about 200 mM. In some embodiments, the potassium chloride is present at a concentration of about 210 mM. In some embodiments, the potassium chloride is present at a concentration of about 220 mM. In some embodiments, the potassium chloride is present at a concentration of about 230 mM. In some embodiments, the potassium chloride is present at a concentration of about 240 mM. In some embodiments, the potassium chloride is present at a concentration of about 250 mM. In some embodiments, the potassium chloride is present at a concentration of about 260 mM. In some embodiments, the potassium chloride is present at a concentration of about 270 mM. In some embodiments, the potassium chloride is present at a concentration of about 280 mM. In some embodiments, the potassium chloride is present at a concentration of about 290 mM. In some embodiments, the potassium chloride is present at a concentration of about 300 mM. In some embodiments, the potassium chloride is present at a concentration of about 310 mM. In some embodiments, the potassium chloride is present at a concentration of about 320 mM. In some embodiments, the potassium chloride is present at a concentration of about 330 mM. In some embodiments, the potassium chloride is present at a concentration of about 340 mM. In some embodiments, the potassium chloride is present at a concentration of about 350 mM. In some embodiments, the potassium chloride is present at a concentration of about 360 mM. In some embodiments, the potassium chloride is present at a concentration of about 370 mM. In some embodiments, the potassium chloride is present at a concentration of about 380 mM. In some embodiments, the potassium chloride is present at a concentration of about 390 mM. In some embodiments, the potassium chloride is present at a concentration of about 400 mM. In some embodiments, the potassium chloride is present at a concentration of about 410 mM. In some embodiments, the potassium chloride is present at a concentration of about 420 mM. In some embodiments, the potassium chloride is present at a concentration of about 430 mM. In some embodiments, the potassium chloride is present at a concentration of about 440 mM. In some embodiments, the potassium chloride is present at a concentration of about 450 mM. In some embodiments, the potassium chloride is present at a concentration of about 460 mM. In some embodiments, the potassium chloride is present at a concentration of about 470 mM. In some embodiments, the potassium chloride is present at a concentration of about 480 mM. In some embodiments, the potassium chloride is present at a concentration of about 490 mM. In some embodiments, the potassium chloride is present at a concentration of about 500 mM. In some embodiments, the potassium chloride is present at a concentration of about 510 mM. In some embodiments, the potassium chloride is present at a concentration of about 520 mM. In some embodiments, the potassium chloride is present at a concentration of about 530 mM. In some embodiments, the potassium chloride is present at a concentration of about 540 mM. In some embodiments, the potassium chloride is present at a concentration of about 550 mM. In some embodiments, the potassium chloride is present at a concentration of about 560 mM. In some embodiments, the potassium chloride is present at a concentration of about 570 mM. In some embodiments, the potassium chloride is present at a concentration of about 580 mM. In some embodiments, the potassium chloride is present at a concentration of about 590 mM. In some embodiments, the potassium chloride is present at a concentration of about 600 mM. In some embodiments, the potassium chloride is present at a concentration of about 610 mM. In some embodiments, the potassium chloride is present at a concentration of about 620 mM. In some embodiments, the potassium chloride is present at a concentration of about 630 mM. In some embodiments, the potassium chloride is present at a concentration of about 640 mM. In some embodiments, the potassium chloride is present at a concentration of about 650 mM. In some embodiments, the potassium chloride is present at a concentration of about 660 mM. In some embodiments, the potassium chloride is present at a concentration of about 670 mM. In some embodiments, the potassium chloride is present at a concentration of about 680 mM. In some embodiments, the potassium chloride is present at a concentration of about 690 mM. In some embodiments, the potassium chloride is present at a concentration of about 700 mM. In some embodiments, the potassium chloride is present at a concentration of about 710 mM. In some embodiments, the potassium chloride is present at a concentration of about 720 mM. In some embodiments, the potassium chloride is present at a concentration of about 730 mM. In some embodiments, the potassium chloride is present at a concentration of about 740 mM. In some embodiments, the potassium chloride is present at a concentration of about 750 mM. In some embodiments, the potassium chloride is present at a concentration of about 760 mM. In some embodiments, the potassium chloride is present at a concentration of about 770 mM. In some embodiments, the potassium chloride is present at a concentration of about 780 mM. In some embodiments, the potassium chloride is present at a concentration of about 790 mM. In some embodiments, the potassium chloride is present at a concentration of about 800 mM.

In some embodiments, the pharmaceutically acceptable salt is magnesium chloride.

In some embodiments, the magnesium chloride is present at a concentration of about 100 mM to about 800 mM, or any value or range in-between. In some embodiments, the magnesium chloride is present at a concentration of about 100 mM to about 700 mM. In some embodiments, the magnesium chloride is present at a concentration of about 100 mM to about 600 mM. In some embodiments, the magnesium chloride is present at a concentration of about 100 mM to about 500 mM. In some embodiments, the magnesium chloride is present at a concentration of about 100 mM to about 400 mM. In some embodiments, the magnesium chloride is present at a concentration of about 100 mM to about 300 mM. In some embodiments, the magnesium chloride is present at a concentration of about 100 mM to about 250 mM. In some embodiments, the magnesium chloride is present at a concentration of about 100 mM to about 200 mM. In some embodiments, the magnesium chloride is present at a concentration of about 100 mM to about 175 mM. In some embodiments, the magnesium chloride is present at a concentration of about 100 mM to about 150 mM. In some embodiments, the magnesium chloride is present at a concentration of about 100 mM to about 125 mM.

In some embodiments, the magnesium chloride is present at a concentration of about 100 mM to about 800 mM. In some embodiments, the magnesium chloride is present at a concentration of about 125 mM to about 800 mM. In some embodiments, the magnesium chloride is present at a concentration of about 150 mM to about 800 mM. In some embodiments, the magnesium chloride is present at a concentration of about 175 mM to about 800 mM. In some embodiments, the magnesium chloride is present at a concentration of about 200 mM to about 800 mM. In some embodiments, the magnesium chloride is present at a concentration of about 250 mM to about 800 mM. In some embodiments, the magnesium chloride is present at a concentration of about 300 mM to about 800 mM. In some embodiments, the magnesium chloride is present at a concentration of about 350 mM to about 800 mM. In some embodiments, the magnesium chloride is present at a concentration of about 400 mM to about 800 mM. In some embodiments, the magnesium chloride is present at a concentration of about 500 mM to about 800 mM. In some embodiments, the magnesium chloride is present at a concentration of about 600 mM to about 800 mM. In some embodiments, the magnesium chloride is present at a concentration of about 700 mM to about 800 mM.

In some embodiments, the magnesium chloride is present at a concentration of about 100 mM. In some embodiments, the magnesium chloride is present at a concentration of about 110 mM. In some embodiments, the magnesium chloride is present at a concentration of about 120 mM. In some embodiments, the magnesium chloride is present at a concentration of about 130 mM. In some embodiments, the magnesium chloride is present at a concentration of about 140 mM. In some embodiments, the magnesium chloride is present at a concentration of about 150 mM. In some embodiments, the magnesium chloride is present at a concentration of about 160 mM. In some embodiments, the magnesium chloride is present at a concentration of about 170 mM. In some embodiments, the magnesium chloride is present at a concentration of about 180 mM. In some embodiments, the magnesium chloride is present at a concentration of about 190 mM. In some embodiments, the magnesium chloride is present at a concentration of about 200 mM. In some embodiments, the magnesium chloride is present at a concentration of about 210 mM. In some embodiments, the magnesium chloride is present at a concentration of about 220 mM. In some embodiments, the magnesium chloride is present at a concentration of about 230 mM. In some embodiments, the magnesium chloride is present at a concentration of about 240 mM. In some embodiments, the magnesium chloride is present at a concentration of about 250 mM. In some embodiments, the magnesium chloride is present at a concentration of about 260 mM. In some embodiments, the magnesium chloride is present at a concentration of about 270 mM. In some embodiments, the magnesium chloride is present at a concentration of about 280 mM. In some embodiments, the magnesium chloride is present at a concentration of about 290 mM. In some embodiments, the magnesium chloride is present at a concentration of about 300 mM. In some embodiments, the magnesium chloride is present at a concentration of about 310 mM. In some embodiments, the magnesium chloride is present at a concentration of about 320 mM. In some embodiments, the magnesium chloride is present at a concentration of about 330 mM. In some embodiments, the magnesium chloride is present at a concentration of about 340 mM. In some embodiments, the magnesium chloride is present at a concentration of about 350 mM. In some embodiments, the magnesium chloride is present at a concentration of about 360 mM. In some embodiments, the magnesium chloride is present at a concentration of about 370 mM. In some embodiments, the magnesium chloride is present at a concentration of about 380 mM. In some embodiments, the magnesium chloride is present at a concentration of about 390 mM. In some embodiments, the magnesium chloride is present at a concentration of about 400 mM. In some embodiments, the magnesium chloride is present at a concentration of about 410 mM. In some embodiments, the magnesium chloride is present at a concentration of about 420 mM. In some embodiments, the magnesium chloride is present at a concentration of about 430 mM. In some embodiments, the magnesium chloride is present at a concentration of about 440 mM. In some embodiments, the magnesium chloride is present at a concentration of about 450 mM. In some embodiments, the magnesium chloride is present at a concentration of about 460 mM. In some embodiments, the magnesium chloride is present at a concentration of about 470 mM. In some embodiments, the magnesium chloride is present at a concentration of about 480 mM. In some embodiments, the magnesium chloride is present at a concentration of about 490 mM. In some embodiments, the magnesium chloride is present at a concentration of about 500 mM. In some embodiments, the magnesium chloride is present at a concentration of about 510 mM. In some embodiments, the magnesium chloride is present at a concentration of about 520 mM. In some embodiments, the magnesium chloride is present at a concentration of about 530 mM. In some embodiments, the magnesium chloride is present at a concentration of about 540 mM. In some embodiments, the magnesium chloride is present at a concentration of about 550 mM. In some embodiments, the magnesium chloride is present at a concentration of about 560 mM. In some embodiments, the magnesium chloride is present at a concentration of about 570 mM. In some embodiments, the magnesium chloride is present at a concentration of about 580 mM. In some embodiments, the magnesium chloride is present at a concentration of about 590 mM. In some embodiments, the magnesium chloride is present at a concentration of about 600 mM. In some embodiments, the magnesium chloride is present at a concentration of about 610 mM. In some embodiments, the magnesium chloride is present at a concentration of about 620 mM. In some embodiments, the magnesium chloride is present at a concentration of about 630 mM. In some embodiments, the magnesium chloride is present at a concentration of about 640 mM. In some embodiments, the magnesium chloride is present at a concentration of about 650 mM. In some embodiments, the magnesium chloride is present at a concentration of about 660 mM. In some embodiments, the magnesium chloride is present at a concentration of about 670 mM. In some embodiments, the magnesium chloride is present at a concentration of about 680 mM. In some embodiments, the magnesium chloride is present at a concentration of about 690 mM. In some embodiments, the magnesium chloride is present at a concentration of about 700 mM. In some embodiments, the magnesium chloride is present at a concentration of about 710 mM. In some embodiments, the magnesium chloride is present at a concentration of about 720 mM. In some embodiments, the magnesium chloride is present at a concentration of about 730 mM. In some embodiments, the magnesium chloride is present at a concentration of about 740 mM. In some embodiments, the magnesium chloride is present at a concentration of about 750 mM. In some embodiments, the magnesium chloride is present at a concentration of about 760 mM. In some embodiments, the magnesium chloride is present at a concentration of about 770 mM. In some embodiments, the magnesium chloride is present at a concentration of about 780 mM. In some embodiments, the magnesium chloride is present at a concentration of about 790 mM. In some embodiments, the magnesium chloride is present at a concentration of about 800 mM.

In some embodiments, the pharmaceutically acceptable salt is calcium chloride.

In some embodiments, the calcium chloride is present at a concentration of about 100 mM to about 800 mM, or any value or range in-between. In some embodiments, the calcium chloride is present at a concentration of about 100 mM to about 700 mM. In some embodiments, the calcium chloride is present at a concentration of about 100 mM to about 600 mM. In some embodiments, the calcium chloride is present at a concentration of about 100 mM to about 500 mM. In some embodiments, the calcium chloride is present at a concentration of about 100 mM to about 400 mM. In some embodiments, the calcium chloride is present at a concentration of about 100 mM to about 300 mM. In some embodiments, the calcium chloride is present at a concentration of about 100 mM to about 250 mM. In some embodiments, the calcium chloride is present at a concentration of about 100 mM to about 200 mM. In some embodiments, the calcium chloride is present at a concentration of about 100 mM to about 175 mM. In some embodiments, the calcium chloride is present at a concentration of about 100 mM to about 150 mM. In some embodiments, the calcium chloride is present at a concentration of about 100 mM to about 125 mM.

In some embodiments, the calcium chloride is present at a concentration of about 100 mM to about 800 mM. In some embodiments, the calcium chloride is present at a concentration of about 125 mM to about 800 mM. In some embodiments, the calcium chloride is present at a concentration of about 150 mM to about 800 mM. In some embodiments, the calcium chloride is present at a concentration of about 175 mM to about 800 mM. In some embodiments, the calcium chloride is present at a concentration of about 200 mM to about 800 mM. In some embodiments, the calcium chloride is present at a concentration of about 250 mM to about 800 mM. In some embodiments, the calcium chloride is present at a concentration of about 300 mM to about 800 mM. In some embodiments, the calcium chloride is present at a concentration of about 350 mM to about 800 mM. In some embodiments, the calcium chloride is present at a concentration of about 400 mM to about 800 mM. In some embodiments, the calcium chloride is present at a concentration of about 500 mM to about 800 mM. In some embodiments, the calcium chloride is present at a concentration of about 600 mM to about 800 mM. In some embodiments, the calcium chloride is present at a concentration of about 700 mM to about 800 mM.

In some embodiments, the calcium chloride is present at a concentration of about 100 mM. In some embodiments, the calcium chloride is present at a concentration of about 110 mM. In some embodiments, the calcium chloride is present at a concentration of about 120 mM. In some embodiments, the calcium chloride is present at a concentration of about 130 mM. In some embodiments, the calcium chloride is present at a concentration of about 140 mM. In some embodiments, the calcium chloride is present at a concentration of about 150 mM. In some embodiments, the calcium chloride is present at a concentration of about 160 mM. In some embodiments, the calcium chloride is present at a concentration of about 170 mM. In some embodiments, the calcium chloride is present at a concentration of about 180 mM. In some embodiments, the calcium chloride is present at a concentration of about 190 mM. In some embodiments, the calcium chloride is present at a concentration of about 200 mM. In some embodiments, the calcium chloride is present at a concentration of about 210 mM. In some embodiments, the calcium chloride is present at a concentration of about 220 mM. In some embodiments, the calcium chloride is present at a concentration of about 230 mM. In some embodiments, the calcium chloride is present at a concentration of about 240 mM. In some embodiments, the calcium chloride is present at a concentration of about 250 mM. In some embodiments, the calcium chloride is present at a concentration of about 260 mM. In some embodiments, the calcium chloride is present at a concentration of about 270 mM. In some embodiments, the calcium chloride is present at a concentration of about 280 mM. In some embodiments, the calcium chloride is present at a concentration of about 290 mM. In some embodiments, the calcium chloride is present at a concentration of about 300 mM. In some embodiments, the calcium chloride is present at a concentration of about 310 mM. In some embodiments, the calcium chloride is present at a concentration of about 320 mM. In some embodiments, the calcium chloride is present at a concentration of about 330 mM. In some embodiments, the calcium chloride is present at a concentration of about 340 mM. In some embodiments, the calcium chloride is present at a concentration of about 350 mM. In some embodiments, the calcium chloride is present at a concentration of about 360 mM. In some embodiments, the calcium chloride is present at a concentration of about 370 mM. In some embodiments, the calcium chloride is present at a concentration of about 380 mM. In some embodiments, the calcium chloride is present at a concentration of about 390 mM. In some embodiments, the calcium chloride is present at a concentration of about 400 mM. In some embodiments, the calcium chloride is present at a concentration of about 410 mM. In some embodiments, the calcium chloride is present at a concentration of about 420 mM. In some embodiments, the calcium chloride is present at a concentration of about 430 mM. In some embodiments, the calcium chloride is present at a concentration of about 440 mM. In some embodiments, the calcium chloride is present at a concentration of about 450 mM. In some embodiments, the calcium chloride is present at a concentration of about 460 mM. In some embodiments, the calcium chloride is present at a concentration of about 470 mM. In some embodiments, the calcium chloride is present at a concentration of about 480 mM. In some embodiments, the calcium chloride is present at a concentration of about 490 mM. In some embodiments, the calcium chloride is present at a concentration of about 500 mM. In some embodiments, the calcium chloride is present at a concentration of about 510 mM. In some embodiments, the calcium chloride is present at a concentration of about 520 mM. In some embodiments, the calcium chloride is present at a concentration of about 530 mM. In some embodiments, the calcium chloride is present at a concentration of about 540 mM. In some embodiments, the calcium chloride is present at a concentration of about 550 mM. In some embodiments, the calcium chloride is present at a concentration of about 560 mM. In some embodiments, the calcium chloride is present at a concentration of about 570 mM. In some embodiments, the calcium chloride is present at a concentration of about 580 mM. In some embodiments, the calcium chloride is present at a concentration of about 590 mM. In some embodiments, the calcium chloride is present at a concentration of about 600 mM. In some embodiments, the calcium chloride is present at a concentration of about 610 mM. In some embodiments, the calcium chloride is present at a concentration of about 620 mM. In some embodiments, the calcium chloride is present at a concentration of about 630 mM. In some embodiments, the calcium chloride is present at a concentration of about 640 mM. In some embodiments, the calcium chloride is present at a concentration of about 650 mM. In some embodiments, the calcium chloride is present at a concentration of about 660 mM. In some embodiments, the calcium chloride is present at a concentration of about 670 mM. In some embodiments, the calcium chloride is present at a concentration of about 680 mM. In some embodiments, the calcium chloride is present at a concentration of about 690 mM. In some embodiments, the calcium chloride is present at a concentration of about 700 mM. In some embodiments, the calcium chloride is present at a concentration of about 710 mM. In some embodiments, the calcium chloride is present at a concentration of about 720 mM. In some embodiments, the calcium chloride is present at a concentration of about 730 mM. In some embodiments, the calcium chloride is present at a concentration of about 740 mM. In some embodiments, the calcium chloride is present at a concentration of about 750 mM. In some embodiments, the calcium chloride is present at a concentration of about 760 mM. In some embodiments, the calcium chloride is present at a concentration of about 770 mM. In some embodiments, the calcium chloride is present at a concentration of about 780 mM. In some embodiments, the calcium chloride is present at a concentration of about 790 mM. In some embodiments, the calcium chloride is present at a concentration of about 800 mM.

In some embodiments, the sugar is present at about 1% (w/v) to about 10% (w/v), or any value or range in-between.

In some embodiments, the sugar is present at about 1% (w/v) to about 10% (w/v). In some embodiments, the sugar is present at about 1% (w/v) to about 8% (w/v). In some embodiments, the sugar is present at about 1% (w/v) to about 7% (w/v). In some embodiments, the sugar is present at about 1% (w/v) to about 6% (w/v). In some embodiments, the sugar is present at about 1% (w/v) to about 5% (w/v). In some embodiments, the sugar is present at about 1% (w/v) to about 4% (w/v). In some embodiments, the sugar is present at about 2% (w/v) to about 4% (w/v). In some embodiments, the sugar is present at about 1% (w/v) to about 3% (w/v). In some embodiments, the sugar is present at about 1% (w/v) to about 2% (w/v).

In some embodiments, the sugar is present at about 1% (w/v) to about 10% (w/v). In some embodiments, the sugar is present at about 2% (w/v) to about 10% (w/v). In some embodiments, the sugar is present at about 3% (w/v) to about 10% (w/v). In some embodiments, the sugar is present at about 4% (w/v) to about 10% (w/v). In some embodiments, the sugar is present at about 5% (w/v) to about 10% (w/v). In some embodiments, the sugar is present at about 6% (w/v) to about 10% (w/v). In some embodiments, the sugar is present at about 7% (w/v) to about 10% (w/v). In some embodiments, the sugar is present at about 8% (w/v) to about 10% (w/v). In some embodiments, the sugar is present at about 9% (w/v) to about 10% (w/v).

In some embodiments, the sugar is present at about 1% (w/v). In some embodiments, the sugar is present at about 2% (w/v). In some embodiments, the sugar is present at about 3% (w/v). In some embodiments, the sugar is present at about 4% (w/v). In some embodiments, the sugar is present at about 5% (w/v). In some embodiments, the sugar is present at about 6% (w/v). In some embodiments, the sugar is present at about 7% (w/v). In some embodiments, the sugar is present at about 8% (w/v). In some embodiments, the sugar is present at about 9% (w/v). In some embodiments, the sugar is present at about 10% (w/v).

In some embodiments, the sugar is selected from sucrose, trehalose, mannose, glucose, sorbitol, mannitol, or a combination thereof.

In some embodiments, the sugar is sucrose. In some embodiments, the sugar is trehalose. In some embodiments, the sugar is mannose. In some embodiments, the sugar is glucose. In some embodiments, the sugar is sorbitol. In some embodiments, the sugar is mannitol.

In some embodiments, the sugar is sucrose.

In some embodiments, the sucrose is present at about 1% (w/v) to about 10% (w/v), or any value or range in-between. In some embodiments, the sucrose is present at about 1% (w/v) to about 9% (w/v). In some embodiments, the sucrose is present at about 1% (w/v) to about 8% (w/v). In some embodiments, the sucrose is present at about 1% (w/v) to about 7% (w/v). In some embodiments, the sucrose is present at about 1% (w/v) to about 6% (w/v). In some embodiments, the sucrose is present at about 1% (w/v) to about 5% (w/v). In some embodiments, the sucrose is present at about 1% (w/v) to about 4% (w/v). In some embodiments, the sucrose is present at about 2% (w/v) to about 4% (w/v). In some embodiments, the sucrose is present at about 1% (w/v) to about 3% (w/v). In some embodiments, the sucrose is present at about 1% (w/v) to about 2% (w/v).

In some embodiments, the sucrose is present at about 1% (w/v) to about 10% (w/v). In some embodiments, the sucrose is present at about 2% (w/v) to about 10% (w/v). In some embodiments, the sucrose is present at about 3% (w/v) to about 10% (w/v). In some embodiments, the sucrose is present at about 4% (w/v) to about 10% (w/v). In some embodiments, the sucrose is present at about 5% (w/v) to about 10% (w/v). In some embodiments, the sucrose is present at about 6% (w/v) to about 10% (w/v). In some embodiments, the sucrose is present at about 7% (w/v) to about 10% (w/v). In some embodiments, the sucrose is present at about 8% (w/v) to about 10% (w/v). In some embodiments, the sucrose is present at about 9% (w/v) to about 10% (w/v).

In some embodiments, the sucrose is present at about 1% (w/v). In some embodiments, the sucrose is present at about 2% (w/v). In some embodiments, the sucrose is present at about 3% (w/v). In some embodiments, the sucrose is present at about 4% (w/v). In some embodiments, the sucrose is present at about 5% (w/v). In some embodiments, the sucrose is present at about 6% (w/v). In some embodiments, the sucrose is present at about 7% (w/v). In some embodiments, the sucrose is present at about 8% (w/v). In some embodiments, the sucrose is present at about 9% (w/v). In some embodiments, the sucrose is present at about 10% (w/v).

In some embodiments, the sucrose is present at about 4% (w/v).

In some embodiments, the sugar is trehalose.

In some embodiments, the trehalose is present at about 1% (w/v) to about 10% (w/v), or any value or range in-between. In some embodiments, the trehalose is present at about 1% (w/v) to about 9% (w/v). In some embodiments, the trehalose is present at about 1% (w/v) to about 8% (w/v). In some embodiments, the trehalose is present at about 1% (w/v) to about 7% (w/v). In some embodiments, the trehalose is present at about 1% (w/v) to about 6% (w/v). In some embodiments, the trehalose is present at about 1% (w/v) to about 5% (w/v). In some embodiments, the trehalose is present at about 1% (w/v) to about 4% (w/v). In some embodiments, the trehalose is present at about 2% (w/v) to about 4% (w/v). In some embodiments, the trehalose is present at about 1% (w/v) to about 3% (w/v). In some embodiments, the trehalose is present at about 1% (w/v) to about 2% (w/v).

In some embodiments, the trehalose is present at about 1% (w/v) to about 10% (w/v). In some embodiments, the trehalose is present at about 2% (w/v) to about 10% (w/v). In some embodiments, the trehalose is present at about 3% (w/v) to about 10% (w/v). In some embodiments, the trehalose is present at about 4% (w/v) to about 10% (w/v). In some embodiments, the trehalose is present at about 5% (w/v) to about 10% (w/v). In some embodiments, the trehalose is present at about 6% (w/v) to about 10% (w/v). In some embodiments, the trehalose is present at about 7% (w/v) to about 10% (w/v). In some embodiments, the trehalose is present at about 8% (w/v) to about 10% (w/v). In some embodiments, the trehalose is present at about 9% (w/v) to about 10% (w/v).

In some embodiments, the trehalose is present at about 1% (w/v). In some embodiments, the trehalose is present at about 2% (w/v). In some embodiments, the trehalose is present at about 3% (w/v). In some embodiments, the trehalose is present at about 4% (w/v). In some embodiments, the trehalose is present at about 5% (w/v). In some embodiments, the trehalose is present at about 6% (w/v). In some embodiments, the trehalose is present at about 7% (w/v). In some embodiments, the trehalose is present at about 8% (w/v). In some embodiments, the trehalose is present at about 9% (w/v). In some embodiments, the trehalose is present at about 10% (w/v).

In some embodiments, the sugar is mannose.

In some embodiments, the mannose is present at about 1% (w/v) to about 10% (w/v), or any value or range in-between. In some embodiments, the mannose is present at about 1% (w/v) to about 9% (w/v). In some embodiments, the mannose is present at about 1% (w/v) to about 8% (w/v). In some embodiments, the mannose is present at about 1% (w/v) to about 7% (w/v). In some embodiments, the mannose is present at about 1% (w/v) to about 6% (w/v). In some embodiments, the mannose is present at about 1% (w/v) to about 5% (w/v). In some embodiments, the mannose is present at about 1% (w/v) to about 4% (w/v). In some embodiments, the mannose is present at about 2% (w/v) to about 4% (w/v). In some embodiments, the mannose is present at about 1% (w/v) to about 3% (w/v). In some embodiments, the mannose is present at about 1% (w/v) to about 2% (w/v).

In some embodiments, the mannose is present at about 1% (w/v) to about 10% (w/v). In some embodiments, the mannose is present at about 2% (w/v) to about 10% (w/v). In some embodiments, the mannose is present at about 3% (w/v) to about 10% (w/v). In some embodiments, the mannose is present at about 4% (w/v) to about 10% (w/v). In some embodiments, the mannose is present at about 5% (w/v) to about 10% (w/v). In some embodiments, the mannose is present at about 6% (w/v) to about 10% (w/v). In some embodiments, the mannose is present at about 7% (w/v) to about 10% (w/v). In some embodiments, the mannose is present at about 8% (w/v) to about 10% (w/v). In some embodiments, the mannose is present at about 9% (w/v) to about 10% (w/v).

In some embodiments, the mannose is present at about 1% (w/v). In some embodiments, the mannose is present at about 2% (w/v). In some embodiments, the mannose is present at about 3% (w/v). In some embodiments, the mannose is present at about 4% (w/v). In some embodiments, the mannose is present at about 5% (w/v). In some embodiments, the mannose is present at about 6% (w/v). In some embodiments, the mannose is present at about 7% (w/v). In some embodiments, the mannose is present at about 8% (w/v). In some embodiments, the mannose is present at about 9% (w/v). In some embodiments, the mannose is present at about 10% (w/v).

In some embodiments, the sugar is glucose.

In some embodiments, the glucose is present at about 1% (w/v) to about 10% (w/v), or any value or range in-between. In some embodiments, the glucose is present at about 1% (w/v) to about 9% (w/v). In some embodiments, the glucose is present at about 1% (w/v) to about 8% (w/v). In some embodiments, the glucose is present at about 1% (w/v) to about 7% (w/v). In some embodiments, the glucose is present at about 1% (w/v) to about 6% (w/v). In some embodiments, the glucose is present at about 1% (w/v) to about 5% (w/v). In some embodiments, the glucose is present at about 1% (w/v) to about 4% (w/v). In some embodiments, the glucose is present at about 2% (w/v) to about 4% (w/v). In some embodiments, the glucose is present at about 1% (w/v) to about 3% (w/v). In some embodiments, the glucose is present at about 1% (w/v) to about 2% (w/v).

In some embodiments, the glucose is present at about 1% (w/v) to about 10% (w/v). In some embodiments, the glucose is present at about 2% (w/v) to about 10% (w/v). In some embodiments, the glucose is present at about 3% (w/v) to about 10% (w/v). In some embodiments, the glucose is present at about 4% (w/v) to about 10% (w/v). In some embodiments, the glucose is present at about 5% (w/v) to about 10% (w/v). In some embodiments, the glucose is present at about 6% (w/v) to about 10% (w/v). In some embodiments, the glucose is present at about 7% (w/v) to about 10% (w/v). In some embodiments, the glucose is present at about 8% (w/v) to about 10% (w/v). In some embodiments, the glucose is present at about 9% (w/v) to about 10% (w/v).

In some embodiments, the glucose is present at about 1% (w/v). In some embodiments, the glucose is present at about 2% (w/v). In some embodiments, the glucose is present at about 3% (w/v). In some embodiments, the glucose is present at about 4% (w/v). In some embodiments, the glucose is present at about 5% (w/v). In some embodiments, the glucose is present at about 6% (w/v). In some embodiments, the glucose is present at about 7% (w/v). In some embodiments, the glucose is present at about 8% (w/v). In some embodiments, the glucose is present at about 9% (w/v). In some embodiments, the glucose is present at about 10% (w/v).

In some embodiments, the sugar is sorbitol.

In some embodiments, the sorbitol is present at about 1% (w/v) to about 10% (w/v), or any value or range in-between. In some embodiments, the sorbitol is present at about 1% (w/v) to about 9% (w/v). In some embodiments, the sorbitol is present at about 1% (w/v) to about 8% (w/v). In some embodiments, the sorbitol is present at about 1% (w/v) to about 7% (w/v). In some embodiments, the sorbitol is present at about 1% (w/v) to about 6% (w/v). In some embodiments, the sorbitol is present at about 1% (w/v) to about 5% (w/v). In some embodiments, the sorbitol is present at about 1% (w/v) to about 4% (w/v). In some embodiments, the sorbitol is present at about 2% (w/v) to about 4% (w/v). In some embodiments, the sorbitol is present at about 1% (w/v) to about 3% (w/v). In some embodiments, the sorbitol is present at about 1% (w/v) to about 2% (w/v).

In some embodiments, the sorbitol is present at about 1% (w/v) to about 10% (w/v). In some embodiments, the sorbitol is present at about 2% (w/v) to about 10% (w/v). In some embodiments, the sorbitol is present at about 3% (w/v) to about 10% (w/v). In some embodiments, the sorbitol is present at about 4% (w/v) to about 10% (w/v). In some embodiments, the sorbitol is present at about 5% (w/v) to about 10% (w/v). In some embodiments, the sorbitol is present at about 6% (w/v) to about 10% (w/v). In some embodiments, the sorbitol is present at about 7% (w/v) to about 10% (w/v). In some embodiments, the sorbitol is present at about 8% (w/v) to about 10% (w/v). In some embodiments, the sorbitol is present at about 9% (w/v) to about 10% (w/v).

In some embodiments, the sorbitol is present at about 1% (w/v). In some embodiments, the sorbitol is present at about 2% (w/v). In some embodiments, the sorbitol is present at about 3% (w/v). In some embodiments, the sorbitol is present at about 4% (w/v). In some embodiments, the sorbitol is present at about 5% (w/v). In some embodiments, the sorbitol is present at about 6% (w/v). In some embodiments, the sorbitol is present at about 7% (w/v). In some embodiments, the sorbitol is present at about 8% (w/v). In some embodiments, the sorbitol is present at about 9% (w/v). In some embodiments, the sorbitol is present at about 10% (w/v).

In some embodiments, the sugar is mannitol.

In some embodiments, the mannitol is present at about 1% (w/v) to about 10% (w/v), or any value or range in-between. In some embodiments, the mannitol is present at about 1% (w/v) to about 9% (w/v). In some embodiments, the mannitol is present at about 1% (w/v) to about 8% (w/v). In some embodiments, the mannitol is present at about 1% (w/v) to about 7% (w/v). In some embodiments, the mannitol is present at about 1% (w/v) to about 6% (w/v). In some embodiments, the mannitol is present at about 1% (w/v) to about 5% (w/v). In some embodiments, the mannitol is present at about 1% (w/v) to about 4% (w/v). In some embodiments, the mannitol is present at about 2% (w/v) to about 4% (w/v). In some embodiments, the mannitol is present at about 1% (w/v) to about 3% (w/v). In some embodiments, the mannitol is present at about 1% (w/v) to about 2% (w/v).

In some embodiments, the mannitol is present at about 1% (w/v) to about 10% (w/v). In some embodiments, the mannitol is present at about 2% (w/v) to about 10% (w/v). In some embodiments, the mannitol is present at about 3% (w/v) to about 10% (w/v). In some embodiments, the mannitol is present at about 4% (w/v) to about 10% (w/v). In some embodiments, the mannitol is present at about 5% (w/v) to about 10% (w/v). In some embodiments, the mannitol is present at about 6% (w/v) to about 10% (w/v). In some embodiments, the mannitol is present at about 7% (w/v) to about 10% (w/v). In some embodiments, the mannitol is present at about 8% (w/v) to about 10% (w/v). In some embodiments, the mannitol is present at about 9% (w/v) to about 10% (w/v).

In some embodiments, the mannitol is present at about 1% (w/v). In some embodiments, the mannitol is present at about 2% (w/v). In some embodiments, the mannitol is present at about 3% (w/v). In some embodiments, the mannitol is present at about 4% (w/v). In some embodiments, the mannitol is present at about 5% (w/v). In some embodiments, the mannitol is present at about 6% (w/v). In some embodiments, the mannitol is present at about 7% (w/v). In some embodiments, the mannitol is present at about 8% (w/v). In some embodiments, the mannitol is present at about 9% (w/v). In some embodiments, the mannitol is present at about 10% (w/v).

In some embodiments, the pharmaceutically acceptable salt is sodium chloride and the sugar is sucrose.

In some embodiments, the pharmaceutically acceptable salt is sodium chloride and the sugar is trehalose.

In some embodiments, the pharmaceutically acceptable salt is sodium chloride and the sugar is mannose.

In some embodiments, the pharmaceutically acceptable salt is sodium chloride and the sugar is glucose.

In some embodiments, the pharmaceutically acceptable salt is sodium chloride and the sugar is sorbitol.

In some embodiments, the pharmaceutically acceptable salt is sodium chloride and the sugar is mannitol.

In some embodiments, the pharmaceutically acceptable salt is potassium chloride and the sugar is sucrose.

In some embodiments, the pharmaceutically acceptable salt is potassium chloride and the sugar is trehalose.

In some embodiments, the pharmaceutically acceptable salt is potassium chloride and the sugar is mannose.

In some embodiments, the pharmaceutically acceptable salt is potassium chloride and the sugar is glucose.

In some embodiments, the pharmaceutically acceptable salt is potassium chloride and the sugar is sorbitol.

In some embodiments, the pharmaceutically acceptable salt is potassium chloride and the sugar is mannitol.

In some embodiments, the pharmaceutically acceptable salt is magnesium chloride and the sugar is sucrose.

In some embodiments, the pharmaceutically acceptable salt is magnesium chloride and the sugar is trehalose.

In some embodiments, the pharmaceutically acceptable salt is magnesium chloride and the sugar is mannose.

In some embodiments, the pharmaceutically acceptable salt is magnesium chloride and the sugar is glucose.

In some embodiments, the pharmaceutically acceptable salt is magnesium chloride and the sugar is sorbitol.

In some embodiments, the pharmaceutically acceptable salt is magnesium chloride and the sugar is mannitol.

In some embodiments, the pharmaceutically acceptable salt is calcium chloride and the sugar is sucrose.

In some embodiments, the pharmaceutically acceptable salt is calcium chloride and the sugar is trehalose.

In some embodiments, the pharmaceutically acceptable salt is calcium chloride and the sugar is mannose.

In some embodiments, the pharmaceutically acceptable salt is calcium chloride and the sugar is glucose.

In some embodiments, the pharmaceutically acceptable salt is calcium chloride and the sugar is sorbitol.

In some embodiments, the pharmaceutically acceptable salt is calcium chloride and the sugar is mannitol.

In some embodiments, the protein excipient is selected from, but not limited to, myoglobulin, serum albumin, bovine serum albumin, human serum albumin, immunoglobulin, immunoglobulin fragments, fibronectin, vitronectin, or a combination thereof. In some embodiments, the protein excipient comprises serum albumin. In some embodiments, the protein excipient comprises human serum albumin. In some embodiments, the human serum albumin is recombinant human serum albumin. In some embodiments, the human serum albumin is non-recombinant human serum albumin. In some embodiments, the non-recombinant human serum albumin is US Pharmacopeia (USP) grade non-recombinant human serum albumin. In some embodiments, the non-recombinant human serum albumin is FDA approved non-recombinant human serum albumin.

In some embodiments, the protein excipient is recombinant human serum albumin. In some embodiments, the protein excipient is non-recombinant human serum albumin.

In some embodiments, the protein excipient is present at about 0.1% (w/v) to about 3% (w/v), or any value or range in-between. In some embodiments, the protein excipient is present at about 0.2% (w/v) to about 3% (w/v). In some embodiments, the protein excipient is present at about 0.3% (w/v) to about 3% (w/v). In some embodiments, the protein excipient is present at about 0.4% (w/v) to about 3% (w/v). In some embodiments, the protein excipient is present at about 0.5% (w/v) to about 3% (w/v). In some embodiments, the protein excipient is present at about 0.6% (w/v) to about 3% (w/v). In some embodiments, the protein excipient is present at about 0.7% (w/v) to about 3% (w/v). In some embodiments, the protein excipient is present at about 0.8% (w/v) to about 3% (w/v). In some embodiments, the protein excipient is present at about 0.9% (w/v) to about 3% (w/v). In some embodiments, the protein excipient is present at about 1% (w/v) to about 3% (w/v). In some embodiments, the protein excipient is present at about 1.2% (w/v) to about 3% (w/v). In some embodiments, the protein excipient is present at about 1.4% (w/v) to about 3% (w/v). In some embodiments, the protein excipient is present at about 1.5% (w/v) to about 3% (w/v). In some embodiments, the protein excipient is present at about 1.6% (w/v) to about 3% (w/v). In some embodiments, the protein excipient is present at about 1.8% (w/v) to about 3% (w/v). In some embodiments, the protein excipient is present at about 2% (w/v) to about 3% (w/v). In some embodiments, the protein excipient is present at about 2.2% (w/v) to about 3% (w/v). In some embodiments, the protein excipient is present at about 2.4% (w/v) to about 3% (w/v). In some embodiments, the protein excipient is present at about 2.5% (w/v) to about 3% (w/v). In some embodiments, the protein excipient is present at about 2.6% (w/v) to about 3% (w/v). In some embodiments, the protein excipient is present at about 2.8% (w/v) to about 3% (w/v).

In some embodiments, the protein excipient is present at about 0.1% (w/v) to about 3% (w/v). In some embodiments, the protein excipient is present at about 0.1% (w/v) to about 2.8% (w/v). In some embodiments, the protein excipient is present at about 0.1% (w/v) to about 2.6% (w/v). In some embodiments, the protein excipient is present at about 0.1% (w/v) to about 2.5% (w/v). In some embodiments, the protein excipient is present at about 0.1% (w/v) to about 2.4% (w/v). In some embodiments, the protein excipient is present at about 0.1% (w/v) to about 2.2% (w/v). In some embodiments, the protein excipient is present at about 0.1% (w/v) to about 2% (w/v). In some embodiments, the protein excipient is present at about 0.1% (w/v) to about 1.8% (w/v). In some embodiments, the protein excipient is present at about 0.1% (w/v) to about 1.6% (w/v). In some embodiments, the protein excipient is present at about 0.1% (w/v) to about 1.5% (w/v). In some embodiments, the protein excipient is present at about 0.1% (w/v) to about 1.4% (w/v). In some embodiments, the protein excipient is present at about 0.1% (w/v) to about 1.2% (w/v). In some embodiments, the protein excipient is present at about 0.1% (w/v) to about 1% (w/v). In some embodiments, the protein excipient is present at about 0.1% (w/v) to about 0.9% (w/v). In some embodiments, the protein excipient is present at about 0.1% (w/v) to about 0.8% (w/v). In some embodiments, the protein excipient is present at about 0.1% (w/v) to about 0.7% (w/v). In some embodiments, the protein excipient is present at about 0.1% (w/v) to about 0.6% (w/v). In some embodiments, the protein excipient is present at about 0.1% (w/v) to about 0.5% (w/v). In some embodiments, the protein excipient is present at about 0.1% (w/v) to about 0.4% (w/v). In some embodiments, the protein excipient is present at about 0.1% (w/v) to about 0.3% (w/v). In some embodiments, the protein excipient is present at about 0.1% (w/v) to about 0.2% (w/v).

In some embodiments, the protein excipient is present at about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3% (w/v).

In some embodiments, the protein excipient is present at about 0.1% (w/v). In some embodiments, the protein excipient is present at about 0.2% (w/v). In some embodiments, the protein excipient is present at about 0.3% (w/v). In some embodiments, the protein excipient is present at about 0.4% (w/v). In some embodiments, the protein excipient is present at about 0.5% (w/v). In some embodiments, the protein excipient is present at about 0.6% (w/v). In some embodiments, the protein excipient is present at about 0.7% (w/v). In some embodiments, the protein excipient is present at about 0.8% (w/v). In some embodiments, the protein excipient is present at about 0.9% (w/v). In some embodiments, the protein excipient is present at about 1% (w/v). In some embodiments, the protein excipient is present at about 1.1% (w/v). In some embodiments, the protein excipient is present at about 1.2% (w/v). In some embodiments, the protein excipient is present at about 1.3% (w/v). In some embodiments, the protein excipient is present at about 1.4% (w/v). In some embodiments, the protein excipient is present at about 1.5% (w/v). In some embodiments, the protein excipient is present at about 1.6% (w/v). In some embodiments, the protein excipient is present at about 1.7% (w/v). In some embodiments, the protein excipient is present at about 1.8% (w/v). In some embodiments, the protein excipient is present at about 1.9% (w/v). In some embodiments, the protein excipient is present at about 2.0% (w/v). In some embodiments, the protein excipient is present at about 2.1% (w/v). In some embodiments, the protein excipient is present at about 2.2% (w/v). In some embodiments, the protein excipient is present at about 2.3% (w/v). In some embodiments, the protein excipient is present at about 2.4% (w/v). In some embodiments, the protein excipient is present at about 2.5% (w/v). In some embodiments, the protein excipient is present at about 2.6% (w/v). In some embodiments, the protein excipient is present at about 2.7% (w/v). In some embodiments, the protein excipient is present at about 2.8% (w/v). In some embodiments, the protein excipient is present at about 2.9% (w/v). In some embodiments, the protein excipient is present at about 3.0% (w/v).

In some embodiments, the protein excipient is present at about 0.5%. In some embodiments, the protein excipient is present at about 1%.

In some embodiments, the protein excipient is a recombinant human serum albumin. The recombinant human serum albumin may be produced from any source. Methods of making recombinant human serum albumin are known in the art, and any such method is within the scope of the present application. In some embodiments, human serum albumin is recombinantly produced from a bacteria source (e.g. bacteria cells). In some embodiments, recombinant human serum albumin is recombinantly produced from an insect source (e.g. insect cells). In some embodiments, recombinant human serum albumin is recombinantly produced from a mammalian source (e.g. mammalian cells). In some embodiments, recombinant human serum albumin is recombinantly produced from a plant source. For example, the albumin can be produced from a plant, such as monocot. One non-limiting example of a monocot that can be used is rice. Examples of such production systems are found in U.S. Pat. Nos. 10,618,951; 7,304,208; and 8,686,225, which are hereby incorporated by reference in their entirety. In some embodiments, the albumin comprises a plant glycosyl group, which can be referred to as a plant glycosylated human serum albumin protein.

In some embodiments, the recombinant human serum albumin is any recombinant human serum albumin. In some embodiments, the recombinant human serum albumin is plant glycosylated human serum albumin. In some embodiments, the human serum albumin is free of blood (e.g., human blood) or animal contaminants. This can be achieved by producing the HSA in a plant, such as a monocot or rice as provided for herein. In some embodiments, the recombinant human serum albumin is sold under the names of Exbumin®(recombinant human albumin-Invitria), Cellastim S (recombinant human serum albumin-Invitria), Albagen (recombinant human albumin-Albumin Bioscience), Recombumin (recombinant human albumin-Albumedix), or Optibumin (recombinant human albumin-Invitria). In some embodiments, the recombinant human serum albumin is Exbumin®. In some embodiments, the recombinant human serum albumin is Cellastim S. In some embodiments, the recombinant human serum albumin is Albagen. In some embodiments, the recombinant human serum albumin is Recombumin. In some embodiments, the recombinant human serum albumin is Optibumin.

In some embodiments, the recombinant human serum albumin is present at about 0.1% (w/v) to about 3% (w/v), or any value or range in-between. In some embodiments, the recombinant human serum albumin is present at about 0.2% (w/v) to about 3% (w/v). In some embodiments, the recombinant human serum albumin is present at about 0.3% (w/v) to about 3% (w/v). In some embodiments, the recombinant human serum albumin is present at about 0.4% (w/v) to about 3% (w/v). In some embodiments, the recombinant human serum albumin is present at about 0.5% (w/v) to about 3% (w/v). In some embodiments, the recombinant human serum albumin is present at about 0.6% (w/v) to about 3% (w/v). In some embodiments, the recombinant human serum albumin is present at about 0.7% (w/v) to about 3% (w/v). In some embodiments, the recombinant human serum albumin is present at about 0.8% (w/v) to about 3% (w/v). In some embodiments, the recombinant human serum albumin is present at about 0.9% (w/v) to about 3% (w/v). In some embodiments, the recombinant human serum albumin is present at about 1% (w/v) to about 3% (w/v). In some embodiments, the recombinant human serum albumin is present at about 1.2% (w/v) to about 3% (w/v). In some embodiments, the recombinant human serum albumin is present at about 1.4% (w/v) to about 3% (w/v). In some embodiments, the recombinant human serum albumin is present at about 1.5% (w/v) to about 3% (w/v). In some embodiments, the recombinant human serum albumin is present at about 1.6% (w/v) to about 3% (w/v). In some embodiments, the recombinant human serum albumin is present at about 1.8% (w/v) to about 3% (w/v). In some embodiments, the recombinant human serum albumin is present at about 2% (w/v) to about 3% (w/v). In some embodiments, the recombinant human serum albumin is present at about 2.2% (w/v) to about 3% (w/v). In some embodiments, the recombinant human serum albumin is present at about 2% (w/v) to about 3% (w/v). In some embodiments, the recombinant human serum albumin is present at about 2.4% (w/v) to about 3% (w/v). In some embodiments, the recombinant human serum albumin is present at about 2.5% (w/v) to about 3% (w/v). In some embodiments, the recombinant human serum albumin is present at about 2.6% (w/v) to about 3% (w/v). In some embodiments, the recombinant human serum albumin is present at about 2.8% (w/v) to about 3% (w/v).

In some embodiments, the recombinant human serum albumin is present at about 0.1% (w/v) to about 3% (w/v). In some embodiments, the recombinant human serum albumin is present at about 0.1% (w/v) to about 2.8% (w/v). In some embodiments, the recombinant human serum albumin is present at about 0.1% (w/v) to about 2.6% (w/v). In some embodiments, the recombinant human serum albumin is present at about 0.1% (w/v) to about 2.5% (w/v). In some embodiments, the recombinant human serum albumin is present at about 0.1% (w/v) to about 2.4% (w/v). In some embodiments, the recombinant human serum albumin is present at about 0.1% (w/v) to about 2.2% (w/v). In some embodiments, the recombinant human serum albumin is present at about 0.1% (w/v) to about 2.0% (w/v). In some embodiments, the recombinant human serum albumin is present at about 0.1% (w/v) to about 1.8% (w/v). In some embodiments, the recombinant human serum albumin is present at about 0.1% (w/v) to about 1.6% (w/v). In some embodiments, the recombinant human serum albumin is present at about 0.1% (w/v) to about 1.5% (w/v). In some embodiments, the recombinant human serum albumin is present at about 0.1% (w/v) to about 1.4% (w/v). In some embodiments, the recombinant human serum albumin is present at about 0.1% (w/v) to about 1.2% (w/v). In some embodiments, the recombinant human serum albumin is present at about 0.1% (w/v) to about 1.0% (w/v). In some embodiments, the recombinant human serum albumin is present at about 0.1% (w/v) to about 0.9% (w/v). In some embodiments, the recombinant human serum albumin is present at about 0.1% (w/v) to about 0.8% (w/v). In some embodiments, the recombinant human serum albumin is present at about 0.1% (w/v) to about 0.7% (w/v). In some embodiments, the recombinant human serum albumin is present at about 0.1% (w/v) to about 0.6% (w/v). In some embodiments, the recombinant human serum albumin is present at about 0.1% (w/v) to about 0.5% (w/v). In some embodiments, the recombinant human serum albumin is present at about 0.1% (w/v) to about 0.4% (w/v). In some embodiments, the recombinant human serum albumin is present at about 0.1% (w/v) to about 0.3% (w/v). In some embodiments, the recombinant human serum albumin is present at about 0.1% (w/v) to about 0.2% (w/v).

In some embodiments, the recombinant human serum albumin is present at about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3% (w/v).

In some embodiments, the recombinant human serum albumin is present at about 0.1% (w/v). In some embodiments, the recombinant human serum albumin is present at about 0.2% (w/v). In some embodiments, the recombinant human serum albumin is present at about 0.3% (w/v). In some embodiments, the recombinant human serum albumin is present at about 0.4% (w/v). In some embodiments, the recombinant human serum albumin is present at about 0.5% (w/v). In some embodiments, the recombinant human serum albumin is present at about 0.6% (w/v). In some embodiments, the recombinant human serum albumin is present at about 0.7% (w/v). In some embodiments, the recombinant human serum albumin is present at about 0.8% (w/v). In some embodiments, the recombinant human serum albumin is present at about 0.9% (w/v). In some embodiments, the recombinant human serum albumin is present at about 1% (w/v). In some embodiments, the recombinant human serum albumin is present at about 1.1% (w/v). In some embodiments, the recombinant human serum albumin is present at about 1.2% (w/v). In some embodiments, the recombinant human serum albumin is present at about 1.3% (w/v). In some embodiments, the recombinant human serum albumin is present at about 1.4% (w/v). In some embodiments, the recombinant human serum albumin is present at about 1.5% (w/v). In some embodiments, the recombinant human serum albumin is present at about 1.6% (w/v). In some embodiments, the recombinant human serum albumin is present at about 1.7% (w/v). In some embodiments, the recombinant human serum albumin is present at about 1.8% (w/v). In some embodiments, the recombinant human serum albumin is present at about 1.9% (w/v). In some embodiments, the recombinant human serum albumin is present at about 2% (w/v). In some embodiments, the recombinant human serum albumin is present at about 2.1% (w/v). In some embodiments, the recombinant human serum albumin is present at about 2.2% (w/v). In some embodiments, the recombinant human serum albumin is present at about 2.3% (w/v). In some embodiments, the recombinant human serum albumin is present at about 2.4% (w/v). In some embodiments, the recombinant human serum albumin is present at about 2.5% (w/v). In some embodiments, the recombinant human serum albumin is present at about 2.6% (w/v). In some embodiments, the recombinant human serum albumin is present at about 2.7% (w/v). In some embodiments, the recombinant human serum albumin is present at about 2.8% (w/v). In some embodiments, the recombinant human serum albumin is present at about 2.9% (w/v). In some embodiments, the recombinant human serum albumin is present at about 3% (w/v).

In some embodiments, the recombinant human serum albumin is present at about 0.5% (w/v). In some embodiments, the recombinant human serum albumin is present at about 1% (w/v).

In some embodiments, the protein excipient is a plant recombinant human serum albumin. In some embodiments, the plant recombinant human serum albumin is any plant recombinant human serum albumin as provided for herein. In some embodiments, the plant recombinant human serum albumin is Exbumin®. In some embodiments, the protein excipient is Exbumin®.

In some embodiments, the Exbumin® is present at about 0.1% (w/v) to 3% (w/v), or any value or range in-between. In some embodiments, the Exbumin® is present at about 0.2% (w/v) to about 3% (w/v). In some embodiments, the Exbumin® is present at about 0.3% (w/v) to about 3% (w/v). In some embodiments, the Exbumin® is present at about 0.4% (w/v) to about 3% (w/v). In some embodiments, the Exbumin® is present at about 0.5% (w/v) to about 3% (w/v). In some embodiments, the Exbumin® is present at about 0.6% (w/v) to about 3% (w/v). In some embodiments, the Exbumin® is present at about 0.7% (w/v) to about 3% (w/v). In some embodiments, the Exbumin® is present at about 0.8% (w/v) to about 3% (w/v). In some embodiments, the Exbumin® is present at about 0.9% (w/v) to about 3% (w/v). In some embodiments, the Exbumin® is present at about 1% (w/v) to about 3% (w/v). In some embodiments, the Exbumin® is present at about 1.2% (w/v) to about 3% (w/v). In some embodiments, the Exbumin® is present at about 1.4% (w/v) to about 3% (w/v). In some embodiments, the Exbumin® is present at about 1.5% (w/v) to about 3% (w/v). In some embodiments, the Exbumin® is present at about 1.6% (w/v) to about 3% (w/v). In some embodiments, the Exbumin® is present at about 1.8% (w/v) to about 3% (w/v). In some embodiments, the Exbumin® is present at about 2% (w/v) to about 3% (w/v). In some embodiments, the Exbumin® is present at about 2.2% (w/v) to about 3% (w/v). In some embodiments, the Exbumin® is present at about 2.4% (w/v) to about 3% (w/v). In some embodiments, the Exbumin® is present at about 2.5% (w/v) to about 3% (w/v). In some embodiments, the Exbumin® is present at about 2.6% (w/v) to about 3% (w/v). In some embodiments, the Exbumin® is present at about 2.8% (w/v) to about 3% (w/v).

In some embodiments, the Exbumin® is present at about 0.1% (w/v) to about 3% (w/v). In some embodiments, the Exbumin® is present at about 0.1% (w/v) to about 2.8% (w/v). In some embodiments, the Exbumin® is present at about 0.1% (w/v) to about 2.6% (w/v). In some embodiments, the Exbumin® is present at about 0.1% (w/v) to about 2.5% (w/v). In some embodiments, the Exbumin® is present at about 0.1% (w/v) to about 2.4% (w/v). In some embodiments, the Exbumin® is present at about 0.1% (w/v) to about 2.2% (w/v). In some embodiments, the Exbumin® is present at about 0.1% (w/v) to about 2% (w/v). In some embodiments, the Exbumin® is present at about 0.1% (w/v) to about 1.8% (w/v). In some embodiments, the Exbumin® is present at about 0.1% (w/v) to about 1.6% (w/v). In some embodiments, the Exbumin® is present at about 0.1% (w/v) to about 1.5% (w/v). In some embodiments, the Exbumin® is present at about 0.1% (w/v) to about 1.4% (w/v). In some embodiments, the Exbumin® is present at about 0.1% (w/v) to about 1.2% (w/v). In some embodiments, the Exbumin® is present at about 0.1% (w/v) to about 1% (w/v). In some embodiments, the Exbumin® is present at about 0.1% (w/v) to about 0.9% (w/v). In some embodiments, the Exbumin® is present at about 0.1% (w/v) to about 0.8% (w/v). In some embodiments, the Exbumin® is present at about 0.1% (w/v) to about 0.7% (w/v). In some embodiments, the Exbumin® is present at about 0.1% (w/v) to about 0.6% (w/v). In some embodiments, the Exbumin® is present at about 0.1% (w/v) to about 0.5% (w/v). In some embodiments, the Exbumin® is present at about 0.1% (w/v) to about 0.4% (w/v). In some embodiments, the Exbumin® is present at about 0.1% (w/v) to about 0.3% (w/v). In some embodiments, the Exbumin® is present at about 0.1% (w/v) to about 0.2% (w/v).

In some embodiments, the Exbumin® is present at about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3% (w/v).

In some embodiments, the Exbumin® is present at about 0.1% (w/v). In some embodiments, the Exbumin® is present at about 0.2% (w/v). In some embodiments, the Exbumin® is present at about 0.3% (w/v). In some embodiments, the Exbumin® is present at about 0.4% (w/v). In some embodiments, the Exbumin® is present at about 0.5% (w/v). In some embodiments, the Exbumin® is present at about 0.6% (w/v). In some embodiments, the Exbumin® is present at about 0.7% (w/v). In some embodiments, the Exbumin® is present at about 0.8% (w/v). In some embodiments, the Exbumin® is present at about 0.9% (w/v). In some embodiments, the Exbumin® is present at about 1% (w/v). In some embodiments, the Exbumin® is present at about 1.1% (w/v). In some embodiments, the Exbumin® is present at about 1.2% (w/v). In some embodiments, the Exbumin® is present at about 1.3% (w/v). In some embodiments, the Exbumin® is present at about 1.4% (w/v). In some embodiments, the Exbumin® is present at about 1.5% (w/v). In some embodiments, the Exbumin® is present at about 1.6% (w/v). In some embodiments, the Exbumin® is present at about 1.7% (w/v). In some embodiments, the Exbumin® is present at about 1.8% (w/v). In some embodiments, the Exbumin® is present at about 1.9% (w/v). In some embodiments, the Exbumin® is present at about 2.0% (w/v). In some embodiments, the Exbumin® is present at about 2.1% (w/v). In some embodiments, the Exbumin® is present at about 2.2% (w/v). In some embodiments, the Exbumin® is present at about 2.3% (w/v). In some embodiments, the Exbumin® is present at about 2.4% (w/v). In some embodiments, the Exbumin® is present at about 2.5% (w/v). In some embodiments, the Exbumin® is present at about 2.6% (w/v). In some embodiments, the Exbumin® is present at about 2.7% (w/v). In some embodiments, the Exbumin® is present at about 2.8% (w/v). In some embodiments, the Exbumin® is present at about 2.9% (w/v). In some embodiments, the Exbumin® is present at about 3.0% (w/v).

In some embodiments, Exbumin® is present at about 0.5% (w/v). In some embodiments, Exbumin® is present at about 1% (w/v).

In some embodiments, the protein excipient is non-recombinant human serum albumin. In some embodiments, the non-recombinant human serum albumin is any non-recombinant human serum albumin. In some embodiments, the non-recombinant human serum albumin is FDA approved, USP grade non-recombinant human serum albumin. The FDA approved, USP grade non-recombinant human serum albumin may be obtained via any appropriate commercial vendor, such as but not limited to, Nova, Octapharma, Grifols Bio Supplies, and the like.

In some embodiments, the non-recombinant human serum albumin is present at about 0.1% (w/v) to about 3% (w/v), or any value or range in-between. In some embodiments, the non-recombinant human serum albumin is present at about 0.2% (w/v) to about 3% (w/v). In some embodiments, the non-recombinant human serum albumin is present at about 0.3% (w/v) to about 3% (w/v). In some embodiments, the non-recombinant human serum albumin is present at about 0.4% (w/v) to about 3% (w/v). In some embodiments, the non-recombinant human serum albumin is present at about 0.5% (w/v) to about 3% (w/v). In some embodiments, the non-recombinant human serum albumin is present at about 0.6% (w/v) to about 3% (w/v). In some embodiments, the non-recombinant human serum albumin is present at about 0.7% (w/v) to about 3% (w/v). In some embodiments, the non-recombinant human serum albumin is present at about 0.8% (w/v) to about 3% (w/v). In some embodiments, the non-recombinant human serum albumin is present at about 0.9% (w/v) to about 3% (w/v). In some embodiments, the non-recombinant human serum albumin is present at about 1% (w/v) to about 3% (w/v). In some embodiments, the non-recombinant human serum albumin is present at about 1.2% (w/v) to about 3% (w/v). In some embodiments, the non-recombinant human serum albumin is present at about 1.4% (w/v) to about 3% (w/v). In some embodiments, the non-recombinant human serum albumin is present at about 1.5% (w/v) to about 3% (w/v). In some embodiments, the non-recombinant human serum albumin is present at about 1.6% (w/v) to about 3% (w/v). In some embodiments, the non-recombinant human serum albumin is present at about 1.8% (w/v) to about 3% (w/v). In some embodiments, the non-recombinant human serum albumin is present at about 2% (w/v) to about 3% (w/v). In some embodiments, the non-recombinant human serum albumin is present at about 2.2% (w/v) to about 3% (w/v). In some embodiments, the non-recombinant human serum albumin is present at about 2% (w/v) to about 3% (w/v). In some embodiments, the non-recombinant human serum albumin is present at about 2.4% (w/v) to about 3% (w/v). In some embodiments, the non-recombinant human serum albumin is present at about 2.5% (w/v) to about 3% (w/v). In some embodiments, the non-recombinant human serum albumin is present at about 2.6% (w/v) to about 3% (w/v). In some embodiments, the non-recombinant human serum albumin is present at about 2.8% (w/v) to about 3% (w/v).

In some embodiments, the non-recombinant human serum albumin is present at about 0.1% (w/v) to about 3% (w/v). In some embodiments, the non-recombinant human serum albumin is present at about 0.1% (w/v) to about 2.8% (w/v). In some embodiments, the non-recombinant human serum albumin is present at about 0.1% (w/v) to about 2.6% (w/v). In some embodiments, the non-recombinant human serum albumin is present at about 0.1% (w/v) to about 2.5% (w/v). In some embodiments, the non-recombinant human serum albumin is present at about 0.1% (w/v) to about 2.4% (w/v). In some embodiments, the non-recombinant human serum albumin is present at about 0.1% (w/v) to about 2.2% (w/v). In some embodiments, the non-recombinant human serum albumin is present at about 0.1% (w/v) to about 2.0% (w/v). In some embodiments, the non-recombinant human serum albumin is present at about 0.1% (w/v) to about 1.8% (w/v). In some embodiments, the non-recombinant human serum albumin is present at about 0.1% (w/v) to about 1.6% (w/v). In some embodiments, the non-recombinant human serum albumin is present at about 0.1% (w/v) to about 1.5% (w/v). In some embodiments, the non-recombinant human serum albumin is present at about 0.1% (w/v) to about 1.4% (w/v). In some embodiments, the non-recombinant human serum albumin is present at about 0.1% (w/v) to about 1.2% (w/v). In some embodiments, the non-recombinant human serum albumin is present at about 0.1% (w/v) to about 1.0% (w/v). In some embodiments, the non-recombinant human serum albumin is present at about 0.1% (w/v) to about 0.9% (w/v). In some embodiments, the non-recombinant human serum albumin is present at about 0.1% (w/v) to about 0.8% (w/v). In some embodiments, the non-recombinant human serum albumin is present at about 0.1% (w/v) to about 0.7% (w/v). In some embodiments, the non-recombinant human serum albumin is present at about 0.1% (w/v) to about 0.6% (w/v). In some embodiments, the non-recombinant human serum albumin is present at about 0.1% (w/v) to about 0.5% (w/v). In some embodiments, the non-recombinant human serum albumin is present at about 0.1% (w/v) to about 0.4% (w/v). In some embodiments, the non-recombinant human serum albumin is present at about 0.1% (w/v) to about 0.3% (w/v). In some embodiments, the non-recombinant human serum albumin is present at about 0.1% (w/v) to about 0.2% (w/v).

In some embodiments, the non-recombinant human serum albumin is present at about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3% (w/v).

In some embodiments, the non-recombinant human serum albumin is present at about 0.1% (w/v). In some embodiments, the non-recombinant human serum albumin is present at about 0.2% (w/v). In some embodiments, the non-recombinant human serum albumin is present at about 0.3% (w/v). In some embodiments, the non-recombinant human serum albumin is present at about 0.4% (w/v). In some embodiments, the non-recombinant human serum albumin is present at about 0.5% (w/v). In some embodiments, the non-recombinant human serum albumin is present at about 0.6% (w/v). In some embodiments, the non-recombinant human serum albumin is present at about 0.7% (w/v). In some embodiments, the non-recombinant human serum albumin is present at about 0.8% (w/v). In some embodiments, the non-recombinant human serum albumin is present at about 0.9% (w/v). In some embodiments, the non-recombinant human serum albumin is present at about 1% (w/v). In some embodiments, the non-recombinant human serum albumin is present at about 1.1% (w/v). In some embodiments, the non-recombinant human serum albumin is present at about 1.2% (w/v). In some embodiments, the non-recombinant human serum albumin is present at about 1.3% (w/v). In some embodiments, the non-recombinant human serum albumin is present at about 1.4% (w/v). In some embodiments, the non-recombinant human serum albumin is present at about 1.5% (w/v). In some embodiments, the non-recombinant human serum albumin is present at about 1.6% (w/v). In some embodiments, the non-recombinant human serum albumin is present at about 1.7% (w/v). In some embodiments, the non-recombinant human serum albumin is present at about 1.8% (w/v). In some embodiments, the non-recombinant human serum albumin is present at about 1.9% (w/v). In some embodiments, the non-recombinant human serum albumin is present at about 2% (w/v). In some embodiments, the non-recombinant human serum albumin is present at about 2.1% (w/v). In some embodiments, the non-recombinant human serum albumin is present at about 2.2% (w/v). In some embodiments, the non-recombinant human serum albumin is present at about 2.3% (w/v). In some embodiments, the non-recombinant human serum albumin is present at about 2.4% (w/v). In some embodiments, the non-recombinant human serum albumin is present at about 2.5% (w/v). In some embodiments, the non-recombinant human serum albumin is present at about 2.6% (w/v). In some embodiments, the non-recombinant human serum albumin is present at about 2.7% (w/v). In some embodiments, the non-recombinant human serum albumin is present at about 2.8% (w/v). In some embodiments, the non-recombinant human serum albumin is present at about 2.9% (w/v). In some embodiments, the non-recombinant human serum albumin is present at about 3% (w/v).

In some embodiments, the non-recombinant human serum albumin is present at about 0.5% (w/v). In some embodiments, the non-recombinant human serum albumin is present at about 1% (w/v).

In various embodiments, a pharmaceutical composition comprises: a lentiviral vector; a pharmaceutically acceptable buffer; a pharmaceutically acceptable salt; a sugar; and a protein excipient, wherein the pharmaceutical composition has a pH of about 6.0 to about 8.0.

In various embodiments, a pharmaceutical composition comprises: a lentiviral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In various embodiments, a pharmaceutical composition comprises: a lentiviral vector; a pharmaceutically acceptable buffer at a concentration of about 5 mM to about 50 mM; a pharmaceutically acceptable salt at a concentration of about 100 mM to about 400 mM; a sugar in an amount of about 2% (w/v) to about 5% (w/v); and (v) a protein excipient in an amount of about 0.1% (w/v) to about 2% (w/v), wherein the pharmaceutical composition has a pH of about 6.0 to about 8.0.

In various embodiments, a pharmaceutical composition comprises: a lentiviral vector; a pharmaceutically acceptable buffer at a concentration of about 5 mM to about 40 mM; a pharmaceutically acceptable salt at a concentration of about 100 mM to about 300 mM; a sugar in an amount of about 3% (w/v) to about 5% (w/v); and a protein excipient in an amount of about 0.1% (w/v) to about 1% (w/v), wherein the pharmaceutical composition has a pH of about 6.0 to about 8.0.

In various embodiments, a pharmaceutical composition comprises: a lentiviral vector; a pharmaceutically acceptable buffer at a concentration of about 10 mM to about 30 mM; a pharmaceutically acceptable salt at a concentration of about 100 mM to about 200 mM; a sugar in an amount of about 3% (w/v) to about 5% (w/v); and a protein excipient in an amount of about 0.1% (w/v) to about 1% (w/v), wherein the pharmaceutical composition has a pH of about 6.0 to about 8.0.

In various embodiments, a pharmaceutical composition comprises: a lentiviral vector; a pharmaceutically acceptable buffer at a concentration of about 10 mM to about 30 mM; a pharmaceutically acceptable salt at a concentration of about 100 mM to about 200 mM; a sugar in an amount of about 3% (w/v) to about 5% (w/v); and a protein excipient in an amount of about 0.1% (w/v) to about 1% (w/v), wherein the pharmaceutical composition has a pH of about 6.5 to about 7.5.

In various embodiments, a pharmaceutical composition comprises: a lentiviral vector; a pharmaceutically acceptable buffer at a concentration of about 20 mM; a pharmaceutically acceptable salt at a concentration of about 150 mM; a sugar in an amount of about 4% (w/v); and a protein excipient in an amount of about 0.5% (w/v), wherein the pharmaceutical composition has a pH of about 7.0.

In some embodiments, the lentiviral vector is a pseudotyped lentiviral vector. Exemplary viral structural proteins that may be used for pseudotyping the lentiviral vectors provided for herein may be found at least in WO2023/064884, WO2023/114698, WO2023/114884, WO2023/154858, and WO2024/026284, each of which are incorporated herein by reference in their entirety. In some embodiments, the lentiviral vector is a VSV-G pseudotyped lentivirus. In some embodiments, the VSV-G pseudotyped lentivirus is as provided for herein.

In some embodiments, the VSV-G pseudotyped lentivirus is present at a concentration of about 1.0E⁺⁰⁵ to 1.0E⁺⁰⁹ TUs/mL.

In some embodiments, the VSV-G pseudotyped lentivirus is present at a concentration of about 1.0E⁺⁰⁵ to about 1.0E⁺⁰⁹ TUs/mL, about 1.0E⁺⁰⁵ to about 1.0E⁺⁰⁸ TUs/mL, about 1.0E⁺⁰⁵ to about 1.0E⁺⁰⁷ TUs/mL, about 1.0E⁺⁰⁵ to about 1.0E⁺⁰⁶ TUs/mL, about 1.0E⁺⁰⁶ to about 1.0E⁺⁰⁹ TUs/mL, about 1.0E⁺⁰⁶ to about 1.0E⁺⁰⁸ TUs/mL, about 1.0E⁺⁰⁶ to about 1.0E⁺⁰⁷ TUs/mL, about 1.0E⁺⁰⁷ to about 1.0E⁺⁰⁹ TUs/mL, about 1.0E⁺⁰⁷ to about 1.0E⁺⁰⁸ TUs/mL, or about 1.0E⁺⁰⁸ to about 1.0E⁺⁰⁹ TUs/mL.

In some embodiments, the VSV-G pseudotyped lentivirus is present at a concentration of about 1.0E⁺⁰⁵ to about 1.0E⁺⁰⁹ TUs/mL. In some embodiments, the VSV-G pseudotyped lentivirus is present at a concentration of about 1.0E⁺⁰⁵ to about 1.0E⁺⁰⁸ TUs/mL. In some embodiments, the VSV-G pseudotyped lentivirus is present at a concentration of about 1.0E⁺⁰⁵ to about 1.0E⁺⁰⁷ TUs/mL. In some embodiments, the VSV-G pseudotyped lentivirus is present at a concentration of about 1.0E⁺⁰⁵ to about 1.0E⁺⁰⁶ TUs/mL. In some embodiments, the VSV-G pseudotyped lentivirus is present at a concentration of about 1.0E⁺⁰⁶ to about 1.0E⁺⁰⁹ TUs/mL. In some embodiments, the VSV-G pseudotyped lentivirus is present at a concentration of about 1.0E⁺⁰⁶ to about 1.0E⁺⁰⁸ TUs/mL. In some embodiments, the VSV-G pseudotyped lentivirus is present at a concentration of about 1.0E⁺⁰⁶ to about 1.0E⁺⁰⁷ TUs/mL. In some embodiments, the VSV-G pseudotyped lentivirus is present at a concentration of about 1.0E⁺⁰⁷ to about 1.0E⁺⁰⁹ TUs/mL. In some embodiments, the VSV-G pseudotyped lentivirus is present at a concentration of about 1.0E⁺⁰⁷ to about 1.0E⁺⁰⁸ TUs/mL. In some embodiments, the VSV-G pseudotyped lentivirus is present at a concentration of about 1.0E⁺⁰⁸ to about 1.0E⁺⁰⁹ TUs/mL.

In some embodiments, the VSV-G pseudotyped lentivirus is present at a concentration of at least 1.0E⁺⁰⁵. In some embodiments, the VSV-G pseudotyped lentivirus is present at a concentration of at least 2.0E⁺⁰⁵. In some embodiments, the VSV-G pseudotyped lentivirus is present at a concentration of at least 3.0E⁺⁰⁵. In some embodiments, the VSV-G pseudotyped lentivirus is present at a concentration of at least 4.0E⁺⁰⁵. In some embodiments, the VSV-G pseudotyped lentivirus is present at a concentration of at least 5.0E⁺⁰⁵. In some embodiments, the VSV-G pseudotyped lentivirus is present at a concentration of at least 6.0E⁺⁰⁵. In some embodiments, the VSV-G pseudotyped lentivirus is present at a concentration of at least 7.0E⁺⁰⁵. In some embodiments, the VSV-G pseudotyped lentivirus is present at a concentration of at least 8.0E⁺⁰⁵. In some embodiments, the VSV-G pseudotyped lentivirus is present at a concentration of at least 9.0E⁺⁰⁵. In some embodiments, the VSV-G pseudotyped lentivirus is present at a concentration of at least 1.0E⁺⁰⁶. In some embodiments, the VSV-G pseudotyped lentivirus is present at a concentration of at least 2.0E⁺⁰⁶. In some embodiments, the VSV-G pseudotyped lentivirus is present at a concentration of at least 3.0E⁺⁰⁶. In some embodiments, the VSV-G pseudotyped lentivirus is present at a concentration of at least 4.0E⁺⁰⁶. In some embodiments, the VSV-G pseudotyped lentivirus is present at a concentration of at least 5.0E⁺⁰⁶. In some embodiments, the VSV-G pseudotyped lentivirus is present at a concentration of at least 6.0E⁺⁰⁶. In some embodiments, the VSV-G pseudotyped lentivirus is present at a concentration of at least 7.0E⁺⁰⁶. In some embodiments, the VSV-G pseudotyped lentivirus is present at a concentration of at least 8.0E⁺⁰⁶. In some embodiments, the VSV-G pseudotyped lentivirus is present at a concentration of at least 9.0E⁺⁰⁶. In some embodiments, the VSV-G pseudotyped lentivirus is present at a concentration of at least 1.0E⁺⁰⁷. In some embodiments, the VSV-G pseudotyped lentivirus is present at a concentration of at least 2.0E⁺⁰⁷. In some embodiments, the VSV-G pseudotyped lentivirus is present at a concentration of at least 3.0E⁺⁰⁷. In some embodiments, the VSV-G pseudotyped lentivirus is present at a concentration of at least 4.0E⁺⁰⁷. In some embodiments, the VSV-G pseudotyped lentivirus is present at a concentration of at least 5.0E⁺⁰⁷. In some embodiments, the VSV-G pseudotyped lentivirus is present at a concentration of at least 6.0E⁺⁰⁷. In some embodiments, the VSV-G pseudotyped lentivirus is present at a concentration of at least 7.0E⁺⁰⁷. In some embodiments, the VSV-G pseudotyped lentivirus is present at a concentration of at least 8.0E⁺⁰⁷. In some embodiments, the VSV-G pseudotyped lentivirus is present at a concentration of at least 9.0E⁺⁰⁷. In some embodiments, the VSV-G pseudotyped lentivirus is present at a concentration of at least 1.0E⁺⁰⁸. In some embodiments, the VSV-G pseudotyped lentivirus is present at a concentration of at least 2.0E⁺⁰⁸. In some embodiments, the VSV-G pseudotyped lentivirus is present at a concentration of at least 3.0E⁺⁰⁸. In some embodiments, the VSV-G pseudotyped lentivirus is present at a concentration of at least 4.0E⁺⁰⁸. In some embodiments, the VSV-G pseudotyped lentivirus is present at a concentration of at least 5.0E⁺⁰⁸. In some embodiments, the VSV-G pseudotyped lentivirus is present at a concentration of at least 6.0E⁺⁰⁸. In some embodiments, the VSV-G pseudotyped lentivirus is present at a concentration of at least 7.0E⁺⁰⁸. In some embodiments, the VSV-G pseudotyped lentivirus is present at a concentration of at least 8.0E⁺⁰⁸. In some embodiments, the VSV-G pseudotyped lentivirus is present at a concentration of at least 9.0E⁺⁰⁸. In some embodiments, the VSV-G pseudotyped lentivirus is present at a concentration of at least 1.0E⁺⁰⁹.

In some embodiments, a pharmaceutical composition is provided, the pharmaceutical composition comprising a VSV-G pseudotyped lentivirus; about 10 mM to about 30 mM of L-Histidine; about 100 mM to about 400 mM of sodium chloride; about 1% (w/v) to about 5% (w/v) of sucrose; and about 0.1% (w/v) to about 3% (w/v) of non-recombinant human serum albumin, wherein the pharmaceutical composition has a pH of about 5.5 to about 8.0.

In some embodiments, the VSV-G pseudotyped lentivirus is present at a concentration as provided for herein. In some embodiments, the VSV-G pseudotyped lentivirus is present at a concentration of about 1.0E⁺⁰⁵ to about 1.0E⁺⁰⁹ as provided for herein.

In some embodiments, the L-Histidine is present at a concentration of about 10 mM to about 30 mM, or any value or range therein. In some embodiments, the L-Histidine is present at a concentration of about 15 mM to about 30 mM. In some embodiments, the L-Histidine is present at a concentration of about 20 mM to about 30 mM. In some embodiments, the L-Histidine is present at a concentration of about 25 mM to about 30 mM. In some embodiments, the L-Histidine is present at a concentration of about 10 mM to about 25 mM. In some embodiments, the L-Histidine is present at a concentration of about 10 mM to about 20 mM. In some embodiments, the L-Histidine is present at a concentration of about 10 mM to about 15 mM.

In some embodiments, the L-Histidine is present at a concentration of about 10 mM. In some embodiments, the L-Histidine is present at a concentration of about 11 mM. In some embodiments, the L-Histidine is present at a concentration of about 12 mM. In some embodiments, the L-Histidine is present at a concentration of about 13 mM. In some embodiments, the L-Histidine is present at a concentration of about 14 mM. In some embodiments, the L-Histidine is present at a concentration of about 15 mM. In some embodiments, the L-Histidine is present at a concentration of about 16 mM. In some embodiments, the L-Histidine is present at a concentration of about 17 mM. In some embodiments, the L-Histidine is present at a concentration of about 18 mM. In some embodiments, the L-Histidine is present at a concentration of about 19 mM. In some embodiments, the L-Histidine is present at a concentration of about 20 mM. In some embodiments, the L-Histidine is present at a concentration of about 25 mM. In some embodiments, the L-Histidine is present at a concentration of about 30 mM.

In some embodiments, the pharmaceutical composition has a pH of about 5.5 to about 8, or any value or range therein. In some embodiments, the pharmaceutical composition has a pH of about 6 to about 8. In some embodiments, the pharmaceutical composition has a pH of about 6.5 to about 8. In some embodiments, the pharmaceutical composition has a pH of about 7 to about 8. In some embodiments, the pharmaceutical composition has a pH of about 7.5 to about 8. In some embodiments, the pharmaceutical composition has a pH of about 6 to about 7.5. In some embodiments, the pharmaceutical composition has a pH of about 6 to about 7. In some embodiments, the pharmaceutical composition has a pH of about 6 to about 6.5.

In some embodiments, the pharmaceutical composition has a pH of about 6. In some embodiments, the pharmaceutical composition has a pH of about 6.5. In some embodiments, the pharmaceutical composition has a pH of about 7. In some embodiments, the pharmaceutical composition has a pH of about 7.5. In some embodiments, the pharmaceutical composition has a pH of about 8.

In some embodiments, the L-Histidine is present at about 10 mM to about 25 mM and the pharmaceutical composition has a pH of about 6 to about 7.

In some embodiments, the L-Histidine is present at about 14 mM and the pharmaceutical composition has a pH of about 7.0.

In some embodiments, the L-Histidine is present at about 20 mM and the pharmaceutical composition has a pH of about 7.0.

In some embodiments, the sodium chloride is present at a concentration of about 100 mM to about 400 mM, or any value or range therein. In some embodiments, the sodium chloride is present at a concentration of about 100 mM to about 390 mM. In some embodiments, the sodium chloride is present at a concentration of about 100 mM to about 380 mM. In some embodiments, the sodium chloride is present at a concentration of about 100 mM to about 370 mM. In some embodiments, the sodium chloride is present at a concentration of about 100 mM to about 360 mM. In some embodiments, the sodium chloride is present at a concentration of about 100 mM to about 350 mM. In some embodiments, the sodium chloride is present at a concentration of about 100 mM to about 340 mM. In some embodiments, the sodium chloride is present at a concentration of about 100 mM to about 330 mM. In some embodiments, the sodium chloride is present at a concentration of about 100 mM to about 320 mM. In some embodiments, the sodium chloride is present at a concentration of about 100 mM to about 310 mM. In some embodiments, the sodium chloride is present at a concentration of about 100 mM to about 300 mM. In some embodiments, the sodium chloride is present at a concentration of about 100 mM to about 290 mM. In some embodiments, the sodium chloride is present at a concentration of about 100 mM to about 280 mM. In some embodiments, the sodium chloride is present at a concentration of about 100 mM to about 270 mM. In some embodiments, the sodium chloride is present at a concentration of about 100 mM to about 260 mM. In some embodiments, the sodium chloride is present at a concentration of about 100 mM to about 250 mM. In some embodiments, the sodium chloride is present at a concentration of about 100 mM to about 240 mM. In some embodiments, the sodium chloride is present at a concentration of about 100 mM to about 230 mM. In some embodiments, the sodium chloride is present at a concentration of about 100 mM to about 220 mM. In some embodiments, the sodium chloride is present at a concentration of about 100 mM to about 210 mM. In some embodiments, the sodium chloride is present at a concentration of about 100 mM to about 200 mM. In some embodiments, the sodium chloride is present at a concentration of about 100 mM to about 190 mM. In some embodiments, the sodium chloride is present at a concentration of about 100 mM to about 180 mM. In some embodiments, the sodium chloride is present at a concentration of about 100 mM to about 170 mM. In some embodiments, the sodium chloride is present at a concentration of about 100 mM to about 160 mM. In some embodiments, the sodium chloride is present at a concentration of about 100 mM to about 150 mM. In some embodiments, the sodium chloride is present at a concentration of about 100 mM to about 140 mM. In some embodiments, the sodium chloride is present at a concentration of about 100 mM to about 130 mM. In some embodiments, the sodium chloride is present at a concentration of about 100 mM to about 120 mM. In some embodiments, the sodium chloride is present at a concentration of about 100 mM to about 110 mM.

In some embodiments, the sodium chloride is present at a concentration of about 110 mM to about 400 mM. In some embodiments, the sodium chloride is present at a concentration of about 120 mM to about 400 mM. In some embodiments, the sodium chloride is present at a concentration of about 130 mM to about 400 mM. In some embodiments, the sodium chloride is present at a concentration of about 140 mM to about 400 mM. In some embodiments, the sodium chloride is present at a concentration of about 150 mM to about 400 mM. In some embodiments, the sodium chloride is present at a concentration of about 160 mM to about 400 mM. In some embodiments, the sodium chloride is present at a concentration of about 170 mM to about 400 mM. In some embodiments, the sodium chloride is present at a concentration of about 180 mM to about 400 mM. In some embodiments, the sodium chloride is present at a concentration of about 190 mM to about 400 mM. In some embodiments, the sodium chloride is present at a concentration of about 200 mM to about 400 mM. In some embodiments, the sodium chloride is present at a concentration of about 210 mM to about 400 mM. In some embodiments, the sodium chloride is present at a concentration of about 220 mM to about 400 mM. In some embodiments, the sodium chloride is present at a concentration of about 230 mM to about 400 mM. In some embodiments, the sodium chloride is present at a concentration of about 240 mM to about 400 mM. In some embodiments, the sodium chloride is present at a concentration of about 250 mM to about 400 mM. In some embodiments, the sodium chloride is present at a concentration of about 260 mM to about 400 mM. In some embodiments, the sodium chloride is present at a concentration of about 270 mM to about 400 mM. In some embodiments, the sodium chloride is present at a concentration of about 280 mM to about 400 mM. In some embodiments, the sodium chloride is present at a concentration of about 290 mM to about 400 mM. In some embodiments, the sodium chloride is present at a concentration of about 300 mM to about 400 mM. In some embodiments, the sodium chloride is present at a concentration of about 310 mM to about 400 mM. In some embodiments, the sodium chloride is present at a concentration of about 320 mM to about 400 mM. In some embodiments, the sodium chloride is present at a concentration of about 330 mM to about 400 mM. In some embodiments, the sodium chloride is present at a concentration of about 340 mM to about 400 mM. In some embodiments, the sodium chloride is present at a concentration of about 350 mM to about 400 mM. In some embodiments, the sodium chloride is present at a concentration of about 360 mM to about 400 mM. In some embodiments, the sodium chloride is present at a concentration of about 370 mM to about 400 mM. In some embodiments, the sodium chloride is present at a concentration of about 380 mM to about 400 mM. In some embodiments, the sodium chloride is present at a concentration of about 390 mM to about 400 mM.

In some embodiments, the sodium chloride is present at a concentration of about 100 mM to about 200 mM. In some embodiments, the sodium chloride is present at a concentration of about 125 mM to about 175 mM.

In some embodiments, the sodium chloride is present at a concentration of about 100 mM. In some embodiments, the sodium chloride is present at a concentration of about 110 mM. In some embodiments, the sodium chloride is present at a concentration of about 120 mM. In some embodiments, the sodium chloride is present at a concentration of about 125 mM. In some embodiments, the sodium chloride is present at a concentration of about 130 mM. In some embodiments, the sodium chloride is present at a concentration of about 140 mM. In some embodiments, the sodium chloride is present at a concentration of about 150 mM. In some embodiments, the sodium chloride is present at a concentration of about 160 mM. In some embodiments, the sodium chloride is present at a concentration of about 170 mM. In some embodiments, the sodium chloride is present at a concentration of about 175 mM. In some embodiments, the sodium chloride is present at a concentration of about 180 mM. In some embodiments, the sodium chloride is present at a concentration of about 190 mM. In some embodiments, the sodium chloride is present at a concentration of about 200 mM.

In some embodiments, sodium chloride is present at a concentration of about 150 mM.

In some embodiments, sucrose is present at about 1% (w/v) to about 5% (w/v), or any value or range therein. In some embodiments, sucrose is present at about 1% (w/v) to about 4% (w/v). In some embodiments, sucrose is present at about 1% (w/v) to about 3% (w/v). In some embodiments, sucrose is present at about 1% (w/v) to about 2% (w/v). In some embodiments, sucrose is present at about 2% (w/v) to about 5% (w/v). In some embodiments, sucrose is present at about 3% (w/v) to about 5% (w/v). In some embodiments, sucrose is present at about 4% (w/v) to about 5% (w/v). In some embodiments, sucrose is present at about 1% (w/v). In some embodiments, sucrose is present at about 2% (w/v). In some embodiments, sucrose is present at about 3% (w/v). In some embodiments, sucrose is present at about 4% (w/v). In some embodiments, sucrose is present at about 5% (w/v).

In some embodiments, sucrose is present at about 4% (w/v).

In some embodiments, the non-recombinant human serum albumin is present at about 0.1% (w/v) to about 3% (w/v), or any value or range in-between. In some embodiments, the non-recombinant human serum albumin is present at about 0.2% (w/v) to about 3% (w/v). In some embodiments, the non-recombinant human serum albumin is present at about 0.3% (w/v) to about 3% (w/v). In some embodiments, the non-recombinant human serum albumin is present at about 0.4% (w/v) to about 3% (w/v). In some embodiments, the non-recombinant human serum albumin is present at about 0.5% (w/v) to about 3% (w/v). In some embodiments, the non-recombinant human serum albumin is present at about 0.6% (w/v) to about 3% (w/v). In some embodiments, the non-recombinant human serum albumin is present at about 0.7% (w/v) to about 3% (w/v). In some embodiments, the non-recombinant human serum albumin is present at about 0.8% (w/v) to about 3% (w/v). In some embodiments, the non-recombinant human serum albumin is present at about 0.9% (w/v) to about 3% (w/v). In some embodiments, the non-recombinant human serum albumin is present at about 1% (w/v) to about 3% (w/v). In some embodiments, the non-recombinant human serum albumin is present at about 1.1% (w/v) to about 3% (w/v). In some embodiments, the non-recombinant human serum albumin is present at about 1.2% (w/v) to about 3% (w/v). In some embodiments, the non-recombinant human serum albumin is present at about 1.3% (w/v) to about 3% (w/v). In some embodiments, the non-recombinant human serum albumin is present at about 1.4% (w/v) to about 3% (w/v). In some embodiments, the non-recombinant human serum albumin is present at about 1.5% (w/v) to about 3% (w/v). In some embodiments, the non-recombinant human serum albumin is present at about 1.6% (w/v) to about 3% (w/v). In some embodiments, the non-recombinant human serum albumin is present at about 1.7% (w/v) to about 3% (w/v). In some embodiments, the non-recombinant human serum albumin is present at about 1.8% (w/v) to about 3% (w/v). In some embodiments, the non-recombinant human serum albumin is present at about 1.9% (w/v) to about 3% (w/v). In some embodiments, the non-recombinant human serum albumin is present at about 2% (w/v) to about 3% (w/v). In some embodiments, the non-recombinant human serum albumin is present at about 2.1% (w/v) to about 3% (w/v). In some embodiments, the non-recombinant human serum albumin is present at about 2.2% (w/v) to about 3% (w/v). In some embodiments, the non-recombinant human serum albumin is present at about 2.3% (w/v) to about 3% (w/v). In some embodiments, the non-recombinant human serum albumin is present at about 2.4% (w/v) to about 3% (w/v). In some embodiments, the non-recombinant human serum albumin is present at about 2.5% (w/v) to about 3% (w/v). In some embodiments, the non-recombinant human serum albumin is present at about 2.6% (w/v) to about 3% (w/v). In some embodiments, the non-recombinant human serum albumin is present at about 2.7% (w/v) to about 3% (w/v). In some embodiments, the non-recombinant human serum albumin is present at about 2.8% (w/v) to about 3% (w/v). In some embodiments, the non-recombinant human serum albumin is present at about 2.9% (w/v) to about 3% (w/v).

In some embodiments, the non-recombinant human serum albumin is present at about 0.1% (w/v) to about 3.0% (w/v). In some embodiments, the non-recombinant human serum albumin is present at about 0.1% (w/v) to about 2.9% (w/v). In some embodiments, the non-recombinant human serum albumin is present at about 0.1% (w/v) to about 2.8% (w/v). In some embodiments, the non-recombinant human serum albumin is present at about 0.1% (w/v) to about 2.7% (w/v). In some embodiments, the non-recombinant human serum albumin is present at about 0.1% (w/v) to about 2.6% (w/v). In some embodiments, the non-recombinant human serum albumin is present at about 0.1% (w/v) to about 2.5% (w/v). In some embodiments, the non-recombinant human serum albumin is present at about 0.1% (w/v) to about 2.4% (w/v). In some embodiments, the non-recombinant human serum albumin is present at about 0.1% (w/v) to about 2.3% (w/v). In some embodiments, the non-recombinant human serum albumin is present at about 0.1% (w/v) to about 2.2% (w/v). In some embodiments, the non-recombinant human serum albumin is present at about 0.1% (w/v) to about 2.1% (w/v). In some embodiments, the non-recombinant human serum albumin is present at about 0.1% (w/v) to about 2.0% (w/v). In some embodiments, the non-recombinant human serum albumin is present at about 0.1% (w/v) to about 1.9% (w/v). In some embodiments, the non-recombinant human serum albumin is present at about 0.1% (w/v) to about 1.8% (w/v). In some embodiments, the non-recombinant human serum albumin is present at about 0.1% (w/v) to about 1.7% (w/v). In some embodiments, the non-recombinant human serum albumin is present at about 0.1% (w/v) to about 1.6% (w/v). In some embodiments, the non-recombinant human serum albumin is present at about 0.1% (w/v) to about 1.5% (w/v). In some embodiments, the non-recombinant human serum albumin is present at about 0.1% (w/v) to about 1.4% (w/v). In some embodiments, the non-recombinant human serum albumin is present at about 0.1% (w/v) to about 1.3% (w/v). In some embodiments, the non-recombinant human serum albumin is present at about 0.1% (w/v) to about 1.2% (w/v). In some embodiments, the non-recombinant human serum albumin is present at about 0.1% (w/v) to about 1.1% (w/v). In some embodiments, the non-recombinant human serum albumin is present at about 0.1% (w/v) to about 1.0% (w/v). In some embodiments, the non-recombinant human serum albumin is present at about 0.1% (w/v) to about 0.9% (w/v). In some embodiments, the non-recombinant human serum albumin is present at about 0.1% (w/v) to about 0.8% (w/v). In some embodiments, the non-recombinant human serum albumin is present at about 0.1% (w/v) to about 0.7% (w/v). In some embodiments, the non-recombinant human serum albumin is present at about 0.1% (w/v) to about 0.6% (w/v). In some embodiments, the non-recombinant human serum albumin is present at about 0.1% (w/v) to about 0.5% (w/v). In some embodiments, the non-recombinant human serum albumin is present at about 0.1% (w/v) to about 0.4% (w/v). In some embodiments, the non-recombinant human serum albumin is present at about 0.1% (w/v) to about 0.3% (w/v). In some embodiments, the non-recombinant human serum albumin is present at about 0.1% (w/v) to about 0.2% (w/v).

In some embodiments, the non-recombinant human serum albumin is present at about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3% (w/v).

In some embodiments, the non-recombinant human serum albumin is present at about 0.1% (w/v). In some embodiments, the non-recombinant human serum albumin is present at about 0.2% (w/v). In some embodiments, the non-recombinant human serum albumin is present at about 0.3% (w/v). In some embodiments, the non-recombinant human serum albumin is present at about 0.4% (w/v). In some embodiments, the non-recombinant human serum albumin is present at about 0.5% (w/v). In some embodiments, the non-recombinant human serum albumin is present at about 0.6% (w/v). In some embodiments, the non-recombinant human serum albumin is present at about 0.7% (w/v). In some embodiments, the non-recombinant human serum albumin is present at about 0.8% (w/v). In some embodiments, the non-recombinant human serum albumin is present at about 0.9% (w/v). In some embodiments, the non-recombinant human serum albumin is present at about 1% (w/v). In some embodiments, the non-recombinant human serum albumin is present at about 1.1% (w/v). In some embodiments, the non-recombinant human serum albumin is present at about 1.2% (w/v). In some embodiments, the non-recombinant human serum albumin is present at about 1.3% (w/v). In some embodiments, the non-recombinant human serum albumin is present at about 1.4% (w/v). In some embodiments, the non-recombinant human serum albumin is present at about 1.5% (w/v). In some embodiments, the non-recombinant human serum albumin is present at about 1.6% (w/v). In some embodiments, the non-recombinant human serum albumin is present at about 1.7% (w/v). In some embodiments, the non-recombinant human serum albumin is present at about 1.8% (w/v). In some embodiments, the non-recombinant human serum albumin is present at about 1.9% (w/v). In some embodiments, the non-recombinant human serum albumin is present at about 2% (w/v). In some embodiments, the non-recombinant human serum albumin is present at about 2.1% (w/v). In some embodiments, the non-recombinant human serum albumin is present at about 2.2% (w/v). In some embodiments, the non-recombinant human serum albumin is present at about 2.3% (w/v). In some embodiments, the non-recombinant human serum albumin is present at about 2.4% (w/v). In some embodiments, the non-recombinant human serum albumin is present at about 2.5% (w/v). In some embodiments, the non-recombinant human serum albumin is present at about 2.6% (w/v). In some embodiments, the non-recombinant human serum albumin is present at about 2.7% (w/v). In some embodiments, the non-recombinant human serum albumin is present at about 2.8% (w/v). In some embodiments, the non-recombinant human serum albumin is present at about 2.9% (w/v). In some embodiments, the non-recombinant human serum albumin is present at about 3% (w/v).

In some embodiments, the non-recombinant human serum albumin is present at about 0.5% (w/v). In some embodiments, the non-recombinant human serum albumin is present at about 1% (w/v).

In some embodiments, the lentiviral vector is a VSV-G pseudotyped lentivirus. In some embodiments, the VSV-G pseudotyped lentivirus is as provided for herein. In some embodiments, the VSV-G pseudotyped lentivirus comprises a VSV-G polypeptide comprising a mutation that corresponds to a mutation at position 182 of SEQ ID NO: 2, as provided for herein. In some embodiments, the VSV-G pseudotyped lentivirus comprises a VSV-G polypeptide comprising a mutation that corresponds to a mutation at position 182, 214, and/or 352 of SEQ ID NO: 2, as provided for herein. In some embodiments, the mutation at position 182 is as provided for herein. In some embodiments, the mutation at position 182 is 182A as provided for herein. In some embodiments, the mutation at position 182 is 182E as provided for herein. In some embodiments, the mutation at position 182 is 182D as provided for herein. In some embodiments, the mutation at position 214 is as provided for herein. In some embodiments, the mutation at position 214 is 214N as provided for herein. In some embodiments, the mutation at position 352 is as provided for herein. In some embodiments, the mutation at position 352 is 352A as provided for herein.

In some embodiments, a pharmaceutical composition is provided, the pharmaceutical composition comprising a VSV-G pseudotyped lentivirus comprising a VSV-G polypeptide comprising a mutation that corresponds to a mutation at position 182, 214, and/or 352 of SEQ ID NO: 2; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a mutation that corresponds to a mutation at position 182, 214, and/or 352 of SEQ ID NO: 2, wherein the mutation at position 182 corresponds to 182A, 182E, or 182D; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a mutation that corresponds to a mutation at position 182, 214, and/or 352 of SEQ ID NO: 2, wherein the mutation at position 182 corresponds to 182E; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a mutation that corresponds to a mutation at position 182, 214, and/or 352 of SEQ ID NO: 2, wherein the mutation at position 182 corresponds to 182D; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a I182E, T214N, and/or T352A mutation as compared to SEQ ID NO: 2; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a I182E, T214N, and T352A mutation as compared to SEQ ID NO: 2; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a I182D, T214N, and/or T352A mutation as compared to SEQ ID NO: 2; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising a I182D, T214N, and T352A mutation as compared to SEQ ID NO: 2; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22, SEQ ID NO: 23, SEQ ID NO: 24, or SEQ ID NO: 25; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22, SEQ ID NO: 23, SEQ ID NO: 24, or SEQ ID NO: 25; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22, SEQ ID NO: 23, SEQ ID NO: 24, or SEQ ID NO: 25; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22, SEQ ID NO: 23, SEQ ID NO: 24, or SEQ ID NO: 25; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5.

In some embodiments, a pharmaceutical composition is provided, the pharmaceutical composition comprising a VSV-G pseudotyped lentivirus comprising a VSV-G polypeptide comprising an amino acid sequence of SEQ ID NO: 22; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising an amino acid sequence of SEQ ID NO: 23; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 23; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 23; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5.

In some embodiments, a pharmaceutical composition is provided, the pharmaceutical composition comprising a VSV-G pseudotyped lentivirus comprising a VSV-G polypeptide comprising an amino acid sequence of SEQ ID NO: 23; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising an amino acid sequence of SEQ ID NO: 24; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 24; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 24; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5.

In some embodiments, a pharmaceutical composition is provided, the pharmaceutical composition comprising a VSV-G pseudotyped lentivirus comprising a VSV-G polypeptide comprising an amino acid sequence of SEQ ID NO: 24; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

In some embodiments, a pharmaceutical composition is provided, the pharmaceutical composition comprising a VSV-G pseudotyped lentivirus comprising a VSV-G polypeptide comprising an amino acid sequence of SEQ ID NO: 25; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 25; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0.

In some embodiments, a pharmaceutical composition is provided, the pharmaceutical composition comprising a VSV-G pseudotyped lentivirus comprising a VSV-G polypeptide comprising an amino acid sequence of SEQ ID NO: 25; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5.

In some embodiments, a pharmaceutical composition is provided, the pharmaceutical composition comprising a VSV-G pseudotyped lentivirus comprising a VSV-G polypeptide comprising an amino acid sequence of SEQ ID NO: 25; L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5. In some embodiments, the non-recombinant human serum albumin is as provided for herein.

As provided for herein, the pharmaceutical composition can have a pH of about 5.5 to about 8.0, or about 6.0 to about 8.0, or any value or range in between. The composition can have a pharmaceutically acceptable buffer, such as those provided for herein, to maintain the pH. When preparing the composition, the pH of the composition can be adjusted to the selected pH by the addition of an acid or base as appropriate. For example, in some embodiments, HCl or another appropriate acid can be added to adjust the pH to make it more acidic, and NaOH or another appropriate base can be added to adjust the pH to be more basic such that the composition has a final pH of about 5.5 to about 8.0, or about 6.0 to about 8.0, or any value or range therein as provided for herein. Appropriate acids are known in the art and include, but are not limited to, hydrochloric acid (HCl), acetic acid (C₂H₄O₂), and sulfuric acid (H₂SO₄). Appropriate bases are known in the art and include, but are not limited to sodium hydroxide (NaOH), potassium hydroxide (KOH), trisodium phosphate (Na₃PO₄), potassium carbonate (K₂CO₃), and sodium carbonate (Na₂CO₃). The acids and bases provided herein are exemplary only and are not meant to be limiting in any way. Appropriate acids and bases are known in the art and any such acid or base is within the scope of the present disclosure. In some embodiments, the acid is hydrochloric acid. In some embodiments, the base is sodium hydroxide.

### Viral Vectors

In some embodiments, the lentiviral vector of the present disclosure is a pseudotyped viral vector. In some embodiments, the pseudotyped viral vector is a VSV-G pseudotyped lentivirus. In some embodiments, the VSV-G psuedotyped lentivirus comprises a VSV-G polypeptide or protein.

In some embodiments, the VSV-G psuedotyped lentivirus comprises a VSV-G protein comprising a mutation at position 198 as compared to SEQ ID NO: 1 or at position 182 as compared to SEQ ID NO: 2. SEQ ID NO: 1 is the full length protein and SEQ ID NO: 2 is the ectodomain of the VSV-G protein. The 16-mer signal peptide of MKCLLYLAFLFIGVNC (SEQ ID NO: 65) as shown at the N-terminus of SEQ ID NO: 1 is cleaved leaving a protein of SEQ ID NO: 2. Thus, although a mutation may be referred to in the context of SEQ ID NO: 2, it should be understood to also be made in the context of SEQ ID NO: 1, which contains the leader sequence, and thus would be a position number that is 16 more than the position recited for SEQ ID NO: 2. In some embodiments, the mutation inhibits or decreases the binding of the VSV-G protein to the LDL receptor (LDL-R). In some embodiments, the mutation is a I182D mutation as compared to SEQ ID NO: 2. In some embodiments, the mutation is a I182E mutation as compared to SEQ ID NO: 2.

In some embodiments, the VSV-G psuedotyped lentivirus comprises a VSV-G protein comprising a mutation at position 198 as compared to SEQ ID NO: 10 or at position 182 as compared to SEQ ID NO: 11. SEQ ID NO: 10 is the full length protein and SEQ ID NO: 11 is the ectodomain of the VSV-G protein. The 16-mer signal peptide of MLSYLIFALVVSPILG (SEQ ID NO: 66) as shown at the N-terminus of SEQ ID NO: 10 is cleaved leaving a protein of SEQ ID NO: 11. Thus, although a mutation may be referred to in the context of SEQ ID NO: 11, it should be understood to also be made in the context of SEQ ID NO: 10, which contains the leader sequence, and thus would be a position number that is 16 more than the position recited for SEQ ID NO: 11. In some embodiments, the mutation inhibits or decreases the binding of the VSV-G protein to the LDL receptor (LDL-R). In some embodiments, the mutation is a T182D mutation as compared to SEQ ID NO: 11. In some embodiments, the mutation is a T182E mutation as compared to SEQ ID NO: 11.

In some embodiments, the VSV-G psuedotyped lentivirus comprises a VSV-G protein comprising a mutation at position 198 as compared to SEQ ID NO: 12 or at position 182 as compared to SEQ ID NO: 13. SEQ ID NO: 12 is the full length protein and SEQ ID NO: 13 is the ectodomain of the VSV-G protein. The 16-mer signal peptide of MLRLFLFCFLALGAHS (SEQ ID NO: 67) as shown at the N-terminus of SEQ ID NO: 12 is cleaved leaving a protein of SEQ ID NO: 13. Thus, although a mutation may be referred to in the context of SEQ ID NO: 13, it should be understood to also be made in the context of SEQ ID NO: 12, which contains the leader sequence, and thus would be a position number that is 16 more than the position recited for SEQ ID NO: 13. In some embodiments, the mutation inhibits or decreases the binding of the VSV-G protein to the LDL receptor (LDL-R). In some embodiments, the mutation is a A182D mutation as compared to SEQ ID NO: 13. In some embodiments, the mutation is a A182E mutation as compared to SEQ ID NO: 13.

In some embodiments, the VSV-G psuedotyped lentivirus comprises a VSV-G protein comprising a mutation at position 203 as compared to SEQ ID NO: 14 or at position 182 as compared to SEQ ID NO: 15. SEQ ID NO: 14 is the full length protein and SEQ ID NO: 15 is the ectodomain of the VSV-G protein. The 21-mer signal peptide of MKMKMVIAGLILCIGILPAIG (SEQ ID NO: 68) as shown at the N-terminus of SEQ ID NO: 14 is cleaved leaving a protein of SEQ ID NO: 15. Thus, although a mutation may be referred to in the context of SEQ ID NO: 15, it should be understood to also be made in the context of SEQ ID NO: 14, which contains the leader sequence, and thus would be a position number that is 21 more than the position recited for SEQ ID NO: 15. In some embodiments, the mutation inhibits or decreases the binding of the VSV-G protein to the LDL receptor (LDL-R). In some embodiments, the mutation is a V182D mutation as compared to SEQ ID NO: 15. In some embodiments, the mutation is a V182E mutation as compared to SEQ ID NO: 15.

In some embodiments, the VSV-G psuedotyped lentivirus comprises a VSV-G protein comprising a mutation at position 199 as compared to SEQ ID NO: 16 or at position 182 as compared to SEQ ID NO: 17. SEQ ID NO: 16 is the full length protein and SEQ ID NO: 17 is the ectodomain of the VSV-G protein. The 17-mer signal peptide of MTPAFILCMLLAGSSWA (SEQ ID NO: 69) as shown at the N-terminus of SEQ ID NO: 16 is cleaved leaving a protein of SEQ ID NO: 17. Thus, although a mutation may be referred to in the context of SEQ ID NO: 17, it should be understood to also be made in the context of SEQ ID NO: 16, which contains the leader sequence, and thus would be a position number that is 17 more than the position recited for SEQ ID NO: 17. In some embodiments, the mutation inhibits or decreases the binding of the VSV-G protein to the LDL receptor (LDL-R). In some embodiments, the mutation is a V182D mutation as compared to SEQ ID NO: 17. In some embodiments, the mutation is a V182E mutation as compared to SEQ ID NO: 17.

In some embodiments, the VSV-G psuedotyped lentivirus comprises a VSV-G protein comprising a mutation at position 199 as compared to SEQ ID NO: 18 or at position 182 as compared to SEQ ID NO: 19. SEQ ID NO: 18 is the full length protein and SEQ ID NO: 19 is the ectodomain of the VSV-G protein. The 17-mer signal peptide of MNFLLLTFIVLPLCSHA (SEQ ID NO: 70) as shown at the N-terminus of SEQ ID NO: 18 is cleaved leaving a protein of SEQ ID NO: 19. Thus, although a mutation may be referred to in the context of SEQ ID NO: 19, it should be understood to also be made in the context of SEQ ID NO: 18, which contains the leader sequence, and thus would be a position number that is 17 more than the position recited for SEQ ID NO: 19. In some embodiments, the mutation inhibits or decreases the binding of the VSV-G protein to the LDL receptor (LDL-R). In some embodiments, the mutation is a V182D mutation as compared to SEQ ID NO: 19. In some embodiments, the mutation is a V182E mutation as compared to SEQ ID NO: 19.

In some embodiments, the VSV-G psuedotyped lentivirus comprises a VSV-G protein comprising a mutation at position 199 as compared to SEQ ID NO: 20 or at position 182 as compared to SEQ ID NO: 21. SEQ ID NO: 20 is the full length protein and SEQ ID NO: 21 is the ectodomain of the VSV-G protein. The 17-mer signal peptide of MLVLYLLLSLLALGAQC (SEQ ID NO: 71) as shown at the N-terminus of SEQ ID NO: 20 is cleaved leaving a protein of SEQ ID NO: 21. Thus, although a mutation may be referred to in the context of SEQ ID NO: 21, it should be understood to also be made in the context of SEQ ID NO: 20, which contains the leader sequence, and thus would be a position number that is 17 more than the position recited for SEQ ID NO: 21. In some embodiments, the mutation inhibits or decreases the binding of the VSV-G protein to the LDL receptor (LDL-R). In some embodiments, the mutation is a I182D mutation as compared to SEQ ID NO: 21. In some embodiments, the mutation is a I182E mutation as compared to SEQ ID NO: 21.

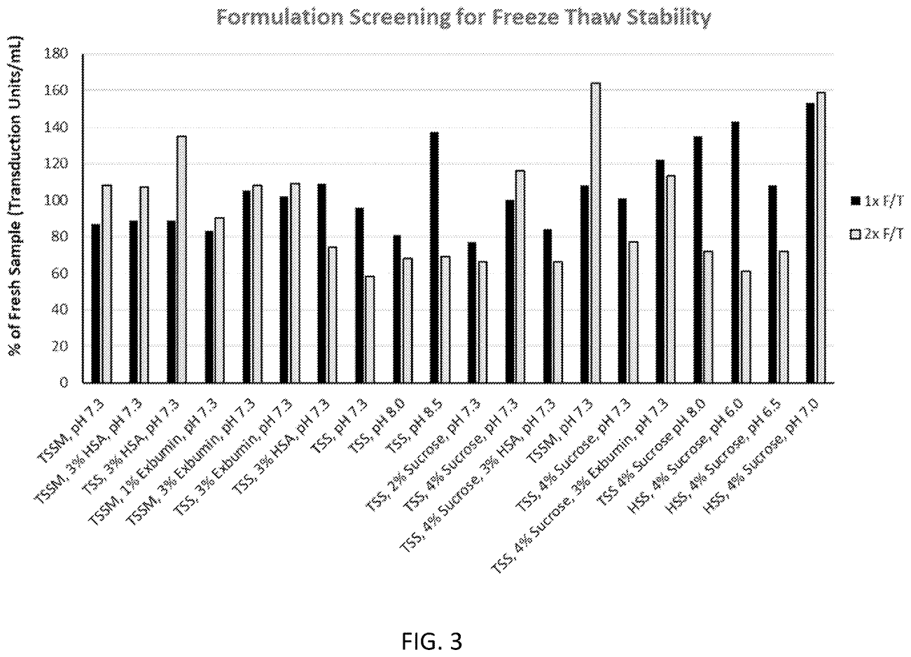

As used herein, when a protein or polypeptide is said to have a mutation as compared to a reference sequence, such comparison is based on an alignment such as using BlastP or ClustalW or ClutalOmega alignment software using default parameters. For example, position 182 can be found in SEQ ID NO: 2 and also as compared to the other strains as illustrated in FIG. 3. FIG. 3 illustrates a clustal alignment of the wild-type sequences of the ectodomains of the various strains of the VSV-G protein. The residue that is bolded and underlined are the residues that align to position 182 of SEQ ID NO: 2 of the various strains. SEQ ID NO: 2 refers to ectodomain of the VSV-G protein of the Indiana strain. SEQ ID NO: 11 refers to ectodomain of the VSV-G protein of the New Jersey strain. SEQ ID NO: 13 refers to ectodomain of the VSV-G protein of the Marraba strain. SEQ ID NO: 15 refers to ectodomain of the VSV-G protein of the Carajas strain. SEQ ID NO: 17 refers to ectodomain of the VSV-G protein of the Alagoa strain. SEQ ID NO: 19 refers to ectodomain of the VSV-G protein of the Cocal strain. SEQ ID NO: 21 refers to ectodomain of the VSV-G protein of the Morreton strain. Accordingly, the residue that aligns to residues 182 as compared to SEQ ID NO: 2 can also be mutated as provided for herein.

In some embodiments, the mutation at position 182 as compared to SEQ ID NO: 2 is not an alanine. In some embodiments, the mutation at position 182 as compared to SEQ ID NO: 2 is not a valine.

In some embodiments, the mutation at position 182 as compared to SEQ ID NO: 2 is I182S, I182H, I182T, I182Q, or I182N. In some embodiments, the mutation at position 182 as compared to SEQ ID NO: 11 is T182S, T182H, T182Q, or T182N. In some embodiments, the mutation at position 182 as compared to SEQ ID NO: 13 is A182S, A182H, A182T, A182Q, or A182N. In some embodiments, the mutation at position 182 as compared to SEQ ID NO: 15 is V182S, V182H, V182T, V182Q, or V182N. In some embodiments, the mutation at position 182 as compared to SEQ ID NO: 17 is V182S, V182H, V182T, V182Q, or V182N. In some embodiments, the mutation at position 182 as compared to SEQ ID NO: 19 is V182S, V182H, V182T, V182Q, or V182N. In some embodiments, the mutation at position 182 as compared to SEQ ID NO: 21 is I182S, I182H, I182T, I182Q, or I182N. In some embodiments, the mutation at position 182 is not a hydrophobic residue. In some embodiments, the mutation at position 182 is a charged residue. In some embodiments, the mutation at position 182 is a negatively charged residue.

Although, the mutations may be described in reference to SEQ ID NO: 1 or SEQ ID NO: 2, which is the VSV-G protein from the Indiana strain, the mutation can also be used in other strains of the VSV-G protein. For example, the mutation can be made in the New Jersey Strain of VSV-G, the Marraba strain of VSV-G, the Carajas strain of VSV-G, the Alagoa strain of VSV-G, the Cocal strain of VSV-G, or the Morreton strain of VSV-G. In some embodiments, the sequences of each are as provided herein. Examples of these can be found, for example in U.S. Patent Application Publication No. 20200216502, which is hereby incorporated by reference. For example, the wild-type full length or ectodomain of the New Jersey Strain of VSV-G are SEQ ID NO: 10 and SEQ ID NO: 11, respectively, the wild-type full length or ectodomain of Marraba strain of VSV-G are SEQ ID NO: 12 and SEQ ID NO: 13, respectively, the wild-type full length or ectodomain of Carajas strain of VSV-G are SEQ ID NO: 14 and SEQ ID NO: 15, respectively, the wild-type full length or ectodomain of Alagoa strain of VSV-G are SEQ ID NO: 16 and SEQ ID NO: 17, respectively, the wild-type full length or ectodomain of Cocal strain of VSV-G are SEQ ID NO: 18 and SEQ ID NO: 19, respectively, or the wild-type full length or ectodomain of Morreton strain of VSV-G are SEQ ID NO: 20 and SEQ ID NO: 21, respectively.

A VSV-G protein comprising a mutation at position 182 as compared to SEQ ID NO: 2 can also comprise other mutations, such as those described in U.S. Patent Application Publication No. 20200216502, which is hereby incorporated by reference in its entirety. For example, the VSV-G protein can comprise a mutation at a position that corresponds to positions of 8, 47, 209 and/or 354 of SEQ ID NO: 2.

In some embodiments, the substitution at position 8 is by any amino acid different from the amino acid indicated at that position in the sequence SEQ ID NO: 2, except Y. In some embodiments, the substitution at position 209 is by any amino acid different from the amino acid indicated at that position in the sequence SEQ ID NO: 2, except H. In some embodiments, the substitution at position 47 is by any amino acid different from the amino acid indicated at that position in the sequence SEQ ID NO: 2, except K or R. In some embodiments, the substitution at position 354 is by any amino acid different from the amino acid indicated at that position in the sequence SEQ ID NO: 2, except K or R.

In some embodiments, the substitution is at position 47 or at position 354, or at both positions 47 and 354 are substituted by A, G, F or Q. In some embodiments, the substitution is A or Q.

In some embodiments, the substitution at position 8 is an alanine, i.e., H8A.

In some embodiments, the substitution at position 47 is Q or N, i.e., K47Q or K47N.

In some embodiments, the protein comprises a mutation (substitution) at position 10. In some embodiments, the substitution/mutation is Q10A, Q10R, or Q10K.

In some embodiments, a protein comprising a mutation at position 182 as compared to SEQ ID NO: 2 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 2 (or SEQ ID NO: 1 if using the full length protein). In some embodiments, the polypeptide comprises a I182D or I182E mutation. In some embodiments, the VSV-G protein comprises a I182S, I182H, I182T, I182Q, or I182N mutation.

In some embodiments, a protein comprising a mutation at position 182 as compared to SEQ ID NO: 11 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 11 (or SEQ ID NO: 10 if using the full length protein). In some embodiments, the polypeptide comprises a T182D or T182E mutation. In some embodiments, the VSV-G protein comprises a T182S, T182H, T182Q, or T182N mutation.

In some embodiments, a protein comprising a mutation at position 182 as compared to SEQ ID NO: 13 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 13 (or SEQ ID NO: 12 if using the full length protein). In some embodiments, the polypeptide comprises a A182D or A182E mutation. In some embodiments, the VSV-G protein comprises a A182S, A182H, A182T, A182Q, or A182N mutation.

In some embodiments, a protein comprising a mutation at position 182 as compared to SEQ ID NO: 15 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 15 (or SEQ ID NO: 14 if using the full length protein). In some embodiments, the polypeptide comprises a V182D or V182E mutation. In some embodiments, the VSV-G protein comprises a V182S, V182H, V182T, V182Q, or V182N mutation.

In some embodiments, a protein comprising a mutation at position 182 as compared to SEQ ID NO: 17 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 17 (or SEQ ID NO: 16 if using the full length protein). In some embodiments, the polypeptide comprises a V182D or V182E mutation. In some embodiments, the VSV-G protein comprises a V182S, V182H, V182T, V182Q, or V182N mutation.

In some embodiments, a protein comprising a mutation at position 182 as compared to SEQ ID NO: 19 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 19 (or SEQ ID NO: 18 if using the full length protein). In some embodiments, the polypeptide comprises a V182D or V182E mutation. In some embodiments, the VSV-G protein comprises a V182S, V182H, V182T, V182Q, or V182N mutation.

In some embodiments, a protein comprising a mutation at position 182 as compared to SEQ ID NO: 21 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 21 (or SEQ ID NO: 20 if using the full length protein). In some embodiments, the polypeptide comprises a I182D or I182E mutation. In some embodiments, the VSV-G protein comprises a I182S, I182H, I182T, I182Q, or I182N mutation.

### Viral Glycoproteins

The mutant VSV-G proteins can be used, for example, to pseudotype a virus, such as, but not limited to a lentivirus. Accordingly, in some embodiments, a viral vector comprising a mutant VSV-G protein as provided herein is provided. In some embodiments, the viral vector comprises a VSV-G protein comprising a mutation at position 198 as compared to SEQ ID NO: 1. In some embodiments, a protein comprising a mutation at position 182 as compared to SEQ ID NO: 2 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 2 (or SEQ ID NO: 1 if using the full length protein). In some embodiments, the polypeptide comprises a I182D or I182E mutation as compared to SEQ ID NO: 2. In some embodiments, the VSV-G protein comprises a I182S, I182H, I182T, I182Q, or I182N mutation.

In some embodiments, the viral vector comprises a VSV-G protein comprising a mutation at position 198 as compared to SEQ ID NO: 10. In some embodiments, a protein comprising a mutation at position 182 as compared to SEQ ID NO: 11 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 11 (or SEQ ID NO: 10 if using the full length protein). In some embodiments, the polypeptide comprises a T182D or T182E mutation as compared to SEQ ID NO: 11. In some embodiments, the VSV-G protein comprises a T182S, T182H, T182Q, or T182N mutation.

In some embodiments, the viral vector comprises a VSV-G protein comprising a mutation at position 198 as compared to SEQ ID NO: 12. In some embodiments, a protein comprising a mutation at position 182 as compared to SEQ ID NO: 13 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 13 (or SEQ ID NO: 12 if using the full length protein). In some embodiments, the polypeptide comprises a A182D or A182E mutation as compared to SEQ ID NO: 13. In some embodiments, the VSV-G protein comprises a A182S, A182H, A182T, A182Q, or A182N mutation.

In some embodiments, the viral vector comprises a VSV-G protein comprising a mutation at position 203 as compared to SEQ ID NO: 14. In some embodiments, a protein comprising a mutation at position 182 as compared to SEQ ID NO: 15 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 15 (or SEQ ID NO: 14 if using the full length protein). In some embodiments, the polypeptide comprises a V182D or V182E mutation as compared to SEQ ID NO: 15. In some embodiments, the VSV-G protein comprises a V182S, V182H, V182T, V182Q, or V182N mutation.

In some embodiments, the viral vector comprises a VSV-G protein comprising a mutation at position 199 as compared to SEQ ID NO: 16. In some embodiments, a protein comprising a mutation at position 182 as compared to SEQ ID NO: 17 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 17 (or SEQ ID NO: 16 if using the full length protein). In some embodiments, the polypeptide comprises a V182D or V182E mutation as compared to SEQ ID NO: 17. In some embodiments, the VSV-G protein comprises a V182S, V182H, V182T, V182Q, or V182N mutation.

In some embodiments, the viral vector comprises a VSV-G protein comprising a mutation at position 199 as compared to SEQ ID NO: 18. In some embodiments, a protein comprising a mutation at position 182 as compared to SEQ ID NO: 19 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 19 (or SEQ ID NO: 18 if using the full length protein). In some embodiments, the polypeptide comprises a V182D or V182E mutation as compared to SEQ ID NO: 19. In some embodiments, the VSV-G protein comprises a V182S, V182H, V182T, V182Q, or V182N mutation.

In some embodiments, the viral vector comprises a VSV-G protein comprising a mutation at position 199 as compared to SEQ ID NO: 20. In some embodiments, a protein comprising a mutation at position 182 as compared to SEQ ID NO: 21 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 21 (or SEQ ID NO: 20 if using the full length protein). In some embodiments, the polypeptide comprises a I182D or I182E mutation as compared to SEQ ID NO: 21. In some embodiments, the VSV-G protein comprises a I182S, I182H, I182T, I182Q, or I182N mutation.

In some embodiments, the VSV-G protein further comprises a mutation at position that corresponds to positions 214 and/or 352 of SEQ ID NO: 2. In some embodiments, the residue that corresponds to position 214 of SEQ ID NO: 2 is T214. In some embodiments, the residue that corresponds to position 352 of SEQ ID NO: is T352. In some embodiments, the VSV-G protein comprises mutation that corresponds to T214N mutation as compared to SEQ ID NO: 2. In some embodiments, the VSV-G protein comprises mutation that corresponds to T352A mutation as compared to SEQ ID NO: 2. In some embodiments, the VSV-G protein comprises a T214N and T352A mutations as compared to SEQ ID NO: 2. These mutations can be combined with any other mutations as provided for herein. In some embodiments, the T214N and/or T352A mutations are combined with the I182E or I182D mutations. In some embodiments, a VSV-G protein comprises an amino acid sequence of SEQ ID NO: 22 and SEQ ID NO: 23, which combines the I182D or I182E, respectively, with the T214N and T352A mutations. The sequences are also illustrated below with the leader sequences, which are removed during protein processing.

VSV-G Protein_I196D, T230N and T368A
mutations (with leader sequence and
adjusted numbering)
(SEQ ID NO: 24)
MKCLLYLAFLFIGVNCKFTIVFPHNQKGNWKNVPSNYHYCPS
SSDLNWHNDLIGTALQVKMPKSHKAIQADGWMCHASKWVTTC
DFRWYGPKYITHSIRSFTPSVEQCKESIEQTKQGTWLNPGFP
PQSCGYATVIDAEAVIVQVTPHHVLVDEYTGEWVDSQFINGK
CSNYICPTVHNSTTWHSDYKVKGLCDSNLDSMDITFFSEDGE
LSSLGKEGTGFRSNYFAYENGGKACKMQYCKHWGVRLPSGVW
FEMADKDLFAAARFPECPEGSSISAPSQTSVDVSLIQDVERI
LDYSLCQETWSKIRAGLPISPVDLSYLAPKNPGTGPAFTIIN
GTLKYFETRYIRVDIAAPILSRMVGMISGTTAERELWDDWAP
YEDVEIGPNGVLRTSSGYKFPLYMIGHGMLDSDLHLSSKAQV
FEHPHIQDAASQLPDDESLFFGDTGLSKNPIELVEGWFSSWK
SSIASFFFIIGLIIGLFLVLRVGIHLCIKLKHTKKRQIYTDI
EMNRLGK
VSV-G Protein_I182D, T214N and T352A
mutations (without leader sequence)
(SEQ ID NO: 22)
KFTIVFPHNQKGNWKNVPSNYHYCPSSSDLNWHNDLIGTALQ
VKMPKSHKAIQADGWMCHASKWVTTCDFRWYGPKYITHSIRS
FTPSVEQCKESIEQTKQGTWLNPGFPPQSCGYATVTDAEAVI
VQVTPHHVLVDEYTGEWVDSQFINGKCSNYICPTVHNSTTWH
SDYKVKGLCDSNLDSMDITFFSEDGELSSLGKEGTGFRSNYF
AYENGGKACKMQYCKHWGVRLPSGVWFEMADKDLFAAARFPE
CPEGSSISAPSQTSVDVSLIQDVERILDYSLCQETWSKIRAG
LPISPVDLSYLAPKNPGTGPAFTIINGTLKYFETRYIRVDIA
APILSRMVGMISGTTAERELWDDWAPYEDVEIGPNGVLRTSS
GYKFPLYMIGHGMLDSDLHLSSKAQVFEHPHIQDAASQLPDD
ESLFFGDTGLSKNPIELVEGWFSSWKSSIASFFFIIGLIIGL
FLVLRVGIHLCIKLKHTKKRQIYTDIEMNRLGK
VSV-G Protein with I196E, T230N and T368A
mutations (with leader sequence and
adjusted numbering)
(SEQ ID NO: 25)
MKCLLYLAFLFIGVNCKFTIVFPHNQKGNWKNVPSNYHYCPS
SSDLNWHNDLIGTALQVKMPKSHKAIQADGWMCHASKWVTTC
DFRWYGPKYITHSIRSFTPSVEQCKESIEQTKQGTWLNPGFP
PQSCGYATVIDAEAVIVQVTPHHVLVDEYTGEWVDSQFINGK
CSNYICPTVHNSTTWHSDYKVKGLCDSNLESMDITFFSEDGE
LSSLGKEGTGFRSNYFAYENGGKACKMQYCKHWGVRLPSGVW
FEMADKDLFAAARFPECPEGSSISAPSQTSVDVSLIQDVERI
LDYSLCQETWSKIRAGLPISPVDLSYLAPKNPGTGPAFTIIN
GTLKYFETRYIRVDIAAPILSRMVGMISGTTAERELWDDWAP
YEDVEIGPNGVLRTSSGYKFPLYMIGHGMLDSDLHLSSKAQV
FEHPHIQDAASQLPDDESLFFGDTGLSKNPIELVEGWFSSWK
SSIASFFFIIGLIIGLFLVLRVGIHLCIKLKHTKKRQIYTDI
EMNRLGK
VSV-G Protein with I182E, T214N and T352A
mutations (without leader sequences)
(SEQ ID NO: 23)
KFTIVFPHNQKGNWKNVPSNYHYCPSSSDLNWHNDLIGTALQ
VKMPKSHKAIQADGWMCHASKWVTTCDFRWYGPKYITHSIRS
FTPSVEQCKESIEQTKQGTWLNPGFPPQSCGYATVTDAEAVI
VQVTPHHVLVDEYTGEWVDSQFINGKCSNYICPTVHNSTTWH
SDYKVKGLCDSNLESMDITFFSEDGELSSLGKEGTGFRSNYF
AYENGGKACKMQYCKHWGVRLPSGVWFEMADKDLFAAARFPE
CPEGSSISAPSQTSVDVSLIQDVERILDYSLCQETWSKIRAG
LPISPVDLSYLAPKNPGTGPAFTIINGILKYFETRYIRVDIA
APILSRMVGMISGTTAERELWDDWAPYEDVEIGPNGVLRTSS
GYKFPLYMIGHGMLDSDLHLSSKAQVFEHPHIQDAASQLPDD
ESLFFGDTGLSKNPIELVEGWFSSWKSSIASFFFIIGLIIGL
FLVLRVGIHLCIKLKHTKKRQIYTDIEMNRLGK

In some embodiments, the VSV-G protein comprising a mutation at position 182 as compared to SEQ ID NO: 2 further comprises a mutation at position that corresponds to positions 38 and/or 320 or SEQ ID NO: 2. In some embodiments, the residue that corresponds to position 38 of SEQ ID NO: 2 is T38. In some embodiments, the residue that corresponds to position 320 of SEQ ID NO: 2 is T320. In some embodiments, the VSV-G protein comprises mutation that corresponds to T38A mutation as compared to SEQ ID NO: 2. In some embodiments, the VSV-G protein comprises mutation that corresponds to T320A mutation as compared to SEQ ID NO: 2. In some embodiments, the VSV-G protein comprises a T38A and T320A mutations as compared to SEQ ID NO: 2. These mutations can be combined with any other mutations as provided for herein.

In some embodiments, the other strains of the VSV-G protein as described herein can further comprise one or more mutations corresponding to any of the other mutations as compared to SEQ ID NO: 2 and as provided for herein. For example, the other strains of the VSV-G protein as described herein can also comprises the mutations that correspond to T38A, T214N, T320A, and/or T352A in SEQ ID NO: 2. In some embodiments, the other strains of the VSV-G protein as described herein can also comprises the mutations that correspond to T214N and/or T352A in SEQ ID NO: 2 and as illustrated in SEQ ID NO: 22 and SEQ ID NO: 23.

In some embodiments, the composition comprises a mutation as described in Hwang et al., Gene Ther 2013 August; 20 (8): 807-15. (Epub 2013 Jan. 31), which is hereby incorporated by reference in its entirety. For example, the mutations can be. at positions 230, 368, 66, and/or 162 that corresponds to SEQ ID NO: 1. The positions will be 16 positions less as compared to SEQ ID NO: 2, when the leader sequence is removed. In some embodiments, the mutations at those positions are, for example, T230N, T368A, K66T, S162T, or any combination thereof. In some embodiments, the VSV-G protein comprises a T230N and a T368A mutation. In some embodiments, the VSV-G polypeptide comprises a K66T, S162T, T230N, and a T368A. These positions are those that correspond to the positions in the full length protein (SEQ ID NO: 1). In some embodiments, the VSV-G protein comprises T230N mutation, a T368A mutation, a K66T mutation, a S162T mutation, or any combination thereof. In some embodiments, the VSV-G protein further comprises one or more mutations in addition to the mutation that corresponds to position 182 of SEQ ID NO: 2, such as those described in U.S. Patent Application Publication No. 20200216502, which is hereby incorporated by reference in its entirety. For example, the VSV-G protein can further comprise a mutation at a position that corresponds to positions of 8, 47, 209 and/or 354 of SEQ ID NO: 2.

In some embodiments, the substitution at position 8 is by any amino acid different from the amino acid indicated at that position in the sequence SEQ ID NO: 2, except Y. In some embodiments, the substitution at position 209 is by any amino acid different from the amino acid indicated at that position in the sequence SEQ ID NO: 2, except H. In some embodiments, the substitution at position 47 is by any amino acid different from the amino acid indicated at that position in the sequence SEQ ID NO: 2, except K or R. In some embodiments, the substitution at position 354 is by any amino acid different from the amino acid indicated at that position in the sequence SEQ ID NO: 2, except K or R. In some embodiments, the substitution is at position 47 or at position 354, or at both positions 47 and 354 are substituted by A, G, For Q. In some embodiments, the substitution is A or Q. In some embodiments, the substitution at position 8 is an alanine, i.e., H8A. In some embodiments, the substitution at position 47 is Q or N, i.e., K47Q or K47N. In some embodiments, the protein comprises a mutation (substitution) at position 10. In some embodiments, the substitution/mutation is Q10A, Q10R, or Q10K.

Additionally, in some embodiments, instead of the VSV-G protein or mutant thereof, the viruses can be pseudotyped with other viral structural proteins. Suitable examples of alternate viral structural proteins may be found at least in WO2023/064884, WO2023/114698, WO2023/114884, and WO2023/154858, each of which are incorporated by reference in their entirety.

### Targeting Moieties

In some embodiments, the viral vector comprises a targeting moiety. The targeting moiety can be used to target the viral vector comprising the mutant VSV-G protein to a cell that expresses the target to which the targeting moiety binds to. In some embodiments, the targeting moiety is an antibody, a scFv antibody, an antigen binding domain, an ankyrin repeat (e.g., DARPIN), a VHH domain antibody, a nanobody, single domain antibody, a FN3 domain, or any combination thereof. The targeting moiety can be attached to the viral surface through an IgG Fc stalk. In some embodiments, the stalk comprises a transmembrane domain. In some embodiments, the transmembrane domain comprises the CD28 transmembrane domain. In some embodiments, the targeting moiety is attached (fused or linked) an envelope glycoprotein G or H of a virus of the Paramyxoviridae family, such as a morbillivirus, such as Measles virus, or a henipavirus, such as Nipah virus, Cedar virus, or Hendra virus. In some embodiments, the targeting moiety can be attached (fused or linked) to a glycoprotein of a virus of the Rhabdoviridae family, such as a vesicular stomatitis New Jersey virus, a vesicular stomatitis Indiana virus, a vesicular stomatitis Alagoas virus, a vesicular stromatitis Maraba virus, a vesicular stomatitis Carajas virus, Parainfluenza virus, Spodoptera frugiperda rhabdovirus isolate Sf G, Drosophila obscura sigmavirus 10A, Wuhan insect virus 7, Perch virus, or Spring viremia of carp virus. In some embodiments, the VSV protein is the mutated proteins, such as those provided for herein. In some embodiments, the targeting moiety is attached to a glycoprotein of a virus of the Filoviridae family, such as Ebola virus or a glycoprotein of a virus of the Arenaviridae family, such as Machupo virus.

In some embodiments, the targeting moiety is a scFv. In some embodiments, the targeting moiety is a single domain antibody. In some embodiments, the targeting moiety is a VHH.

In some embodiments, the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Asialoglycoprotein receptor (ASGPR); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CAIX); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLII; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudin18.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMI; G protein coupled receptor class C group 5 member D (GPRC5D); G protein-coupled receptor 20 (GPR20); GAD; Ganglioside G2 (GD2); Ganglioside GD3 (aNeu5Ac (2-8) aNeu5Ac (2-3) bDGalp (1-4) bDGlcp (1-1) Cer); Ganglioside GM3 (aNeu5Ac (2-3) bDClalp (1-4) bDGlcp (1-1) Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVI-Tax; Human papilloma virus E6 (HPV E6); Human papilloma virus E7 (HPV E7); Human Telomerase reverse transcriptase (hTERT); IgE; IL13Ra2; ILI IRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-IlRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis (Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLACI); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Tim1-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1). In some embodiments, the targeting moiety binds to CD7. In some embodiments, the targeting moiety binds to CD8.

In some embodiments, the targeting moiety binds to a target that is present on a cell, such as an immune cell. In some embodiments, the cell is an immune cell, such as, but not limited to, T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD20+ cancer cell; CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte. In some embodiments, the cell is a T cell. In some embodiments, the cell is a B cell. In some embodiments, the cell is a CD7+ T cell and/or CD8+ T cell.

In some embodiments, the targeting moiety (a polypeptide) can bind to CD7.

In some embodiments, the polypeptide binds to CD7. In some embodiments, the polypeptide that binds to CD7 is an antibody which binds to non-human primate CD7. In some embodiments, the polypeptide that binds to CD7 is an antibody which binds to human CD7. The sequence of human CD7 (UniProtKB P09564) is as follows (SEQ ID NO: 29):

(SEQ ID NO: 29)
MAGPPRLLLLPLLLALARGLPGALAAQEVQQSPHCTTVPVGASVNITCS
TSGGLRGIYLRQLGPQPQDIIYYEDGVVPTTDRRFRGRIDFSGSQDNLT
ITMHRLQLSDTGTYTCQAITEVNVYGSGTLVLVTEEQSQGWHRCSDAPP
RASALPAPPTGSALPDPQTASALPDPPAASALPAALAVISFLLGLGLGV
ACVLARTQIKKLCSWRDKNSAACVVYEDMSHSRCNTLSSPNQYQ

In some embodiments, the CD7 antibody comprises a Fc region. The Fc region can be linked to the heavy or light chain of the antibody. The Fc region may be fused directly to the heavy or light chain of the antibody or may be fused indirectly to the heavy or light chain of the antibody via, for example, a peptide linker as provided for herein. In some embodiments, the Fc region is an IgG Fc. In some embodiments, the IgG is selected from IgG1, IgG2, IgG3, or IgG4. In some embodiments, the IgG fc is IgG1 Fc. In some embodiments, the antibody comprises an Fc constant region of SEQ ID NO: 26 as set forth below:

(SEQ ID NO: 26)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKENWYVDGVEVHNAKTKPREEQYNSTYRVVSVLIVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the IgG fc is IgG2 Fc. In some embodiments, the antibody comprises an Fc constant region of SEQ ID NO: 27 as set forth below:

(SEQ ID NO: 27)
STKGPSVFPLAPCSRSTSESTAALGCLVKDYFPEPVTVSWNSGALTSGVHTFPAVLQ
SSGLYSLSSVVTVPSSSLGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPRE
EQFNSTFRVVSVLTVVHQDWLNGKEYKCKVSNKGLPAPIEKTISKTKGQPREPQVYT
LPPSREEMTKNQVSLTCLVKGFYPSDIAVEWESNGQPENNYKTTPPMLDSDGSFFLY
SKLTVDKSRWQQGNVFSCSVMHEALHNHYTQKSLSLSPGK

In some embodiments, the IgG fc is IgG4 Fc. In some embodiments, the antibody comprises an Fc constant region of SEQ ID NO: 28 as set forth below:

(SEQ ID NO: 28)
STKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFL
GGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK

In some embodiments, the IgG Fc is a variant of an IgG1 Fc protein (SEQ ID NO: 26). In some embodiments, the variant IgG1 Fc protein comprises one or more of the mutations that corresponds to those selected from the group consisting of: L234A, L235A, N297A, P329G, I253A, H310A, and H435A of SEQ ID NO: 26. Any of the mutations L234A, L235A, N297A, P329G, I253A, H310A, and H435A of SEQ ID NO: 26 may be present or absent and the mutations may be combined in any combination. In some embodiments, the variant IgG1 Fc protein comprises a mutation that corresponds to L234A and L235A of SEQ ID NO: 26. In some embodiments, the variant IgG1 Fc protein comprises a mutation that corresponds to N297A of SEQ ID NO: 26. In some embodiments, the variant IgG1 Fc protein comprises a mutation that corresponds to P329G of SEQ ID NO: 26. In some embodiments, the variant IgG1 Fc protein comprises a mutation that corresponds to L234A, L235A, N297A, and P329G of SEQ ID N: 83. In some embodiments, the variant IgG1 Fc protein comprises a mutation that corresponds to I253A of SEQ ID NO: 26. In some embodiments, the variant IgG1 Fc protein comprises a mutation that corresponds to H310A of SEQ ID NO: 26. In some embodiments, the variant IgG1 Fc protein comprises a mutation that corresponds to H435A of SEQ ID NO: 26. In some embodiments, the variant IgG1 Fc protein comprises a mutation that corresponds to I253A, H310A, and H435A of SEQ ID NO: 26.

In some embodiments, the IgG Fc is a variant of an IgG2 Fc protein (SEQ ID NO: 27). In some embodiments, the variant IgG2 Fc protein comprises one or more mutations selected from the group consisting of: N297A, P329G, I253A, H310A, and H435A as those position correspond to SEQ ID NO: 27. Any of the mutations N297A, P329G, I253A, H310A, and H435A of SEQ ID NO: 27 may be present or absent and the mutations may be combined in any combination. In some embodiments, the variant IgG2 Fc protein comprises a mutation that corresponds to N297A of SEQ ID NO: 27. In some embodiments, the variant IgG2 Fc protein comprises a mutation that corresponds to P329G of SEQ ID NO: 27. In some embodiments, the variant IgG2 Fc protein comprises a mutation that corresponds to N297A and P329G of SEQ ID NO: 27. In some embodiments, the variant IgG2 Fc protein comprises a mutation that corresponds to I253A of SEQ ID NO: 27. In some embodiments, the variant IgG2 Fc protein comprises a mutation that corresponds to H310A of SEQ ID NO: 27. In some embodiments, the variant IgG2 Fc protein comprises a mutation that corresponds to H435A of SEQ ID NO: 27. In some embodiments, the variant IgG2 Fc protein comprises a mutation that corresponds to I253A, H310A, and H435A of SEQ ID NO: 27.

In some embodiments, the IgG Fc protein is a variant of an IgG4 Fc protein (SEQ ID NO: 28). In some embodiments, the variant IgG4 Fc protein comprises one or more mutations selected from the group consisting of: S228P, L235E, N297A, P329G, I253A, H310A, and H435A as those positions correspond to SEQ ID NO: 28. Any of the mutations S228P, L235E, N297A, P329G, 1253A, H310A, and H435A of SEQ ID NO: 28 may be present or absent and the mutations may be combined in any combination. In some embodiments, the variant IgG4 Fc protein comprises a mutation that corresponds to S228P of SEQ ID NO: 28. In some embodiments, the variant IgG4 Fc protein comprises a mutation that corresponds to L235E of SEQ ID NO: 28. In some embodiments, the variant IgG4 Fc protein comprises a mutation that corresponds to N297A of SEQ ID NO: 28. In some embodiments, the variant IgG4 Fc protein comprises a mutation that corresponds to P329G of SEQ ID NO: 28. In some embodiments, the variant IgG4 Fc protein comprises a mutation that corresponds to S228P, L235E, N297A, and P329G of SEQ ID NO: 28. In some embodiments, the variant IgG4 Fc protein comprises a mutation that corresponds to I253A of SEQ ID NO: 28. In some embodiments, the variant IgG4 Fc protein comprises a mutation that corresponds to H310A of SEQ ID NO: 28. In some embodiments, the variant IgG4 Fc protein comprises a mutation that corresponds to H435A of SEQ ID NO: 28. In some embodiments, the variant IgG4 Fc protein comprises a mutation that corresponds to I253A, H310A, and H435A of SEQ ID NO: 28.

In some embodiments, the Fc region comprises a variant Fc polypeptide. In some embodiments, the variant Fc polypeptide is a variant Fc polypeptide as provided for in PCT Publication No. WO2024026284 which is hereby incorporated by reference in its entirety. In some embodiments, the variant Fc polypeptide comprises an amino acid sequence that is at least 85%, 90%, 91%, 92%, 93%, 94%, 95%, 96%, 97%, 98%, or 99% identical to the amino acid sequence of SEQ ID NO: 82:

(SEQ ID NO: 82)
EPKSCDKTHTCPPCPAPEAAGGPSVFLFPPKPKDTLMASRTPEVTCVVV
DVSHEDPEVKFNWYVDGVEVHNAKTKPREEQYASTYRVVSVLTVLAQDW
LNGKEYKCKVSNKALGAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVESCSVMHEALHNAYTQKSLSLSPGK

In some embodiments, the variant Fc polypeptide comprises an amino acid sequence of SEQ ID NO: 82.

In some embodiments, the targeting moiety binds to CD7 and comprises an HCDR1, HCDR2, HCDR3, LCDR1, LCDR2, LCDR3, VH, and VL as recited in Table 1 and Table 2 below:

TABLE 1
CD7 targeting moiety CD7AB1 CDR sequences

| Numbering | | | | | | |
|---|---|---|---|---|---|---|
| System | HCDR1 | HCDR2 | HCDR3 | LCDR1 | LCDR2 | LCDR3 |
| Chothia | GYPFTSY | DPNSGD | SPYYSNDN | RASQSIGTS | YASESIS | QQSNSWP |
| | (SEQ ID | (SEQ ID | SMDY | IH (SEQ | (SEQ ID | TT (SEQ |
| | NO: 30) | NO: 31) | (SEQ ID | ID NO: | NO: 34) | ID NO: |
| | | | NO: 32) | 33) | | 35) |
| Kabat | SYWIH | RIDPNSGDT | SEQ ID | SEQ ID | SEQ ID | SEQ ID |
| | (SEQ ID | KYNEKFKN | NO: 32 | NO: 33 | NO: 34 | NO: 35 |
| | NO: 36) | (SEQ ID | | | | |
| | | NO: 37) | | | | |
| IMGT | GYPFTSY | IDPNSGDT | ARSPYYSN | QSIGTS | YA | SEQ ID |
| | W (SEQ | (SEQ ID | DNSMDY | (SEQ ID | | NO: 35 |
| | ID NO: | NO: 39) | (SEQ ID | NO: 41) | | |
| | 38) | | NO: 40) | | | |

TABLE 2
CD7 targeting moiety CD7AB1 Variable Regions

| SEQ ID NO: | AB ID NO: | Region | Sequence |
|---|---|---|---|
| 42 | CD7AB1 | VH | QVQLQQPGAELVKPGASVKLSCKASGYPFTSYWIHWVKQRPGRGLEWL |
| | | | GRIDPNSGDTKYNEKFKNKATLTVDKSSTTAYMQLSSLTSEDSAVYYC |
| | | | ARSPYYSNDNSMDYWGQGTSVTVSS |
| 43 | CD7AB1 | VL | DILLTQSPAILSVSPGERVSFSCRASQSIGTSIHWYQQRINDSPRLLI |
| | | | KYASESISGIPSRFSGSGSGTDFTLSINSVESEDIADYYCQQSNSWPT |
| | | | TFGGGTKLEIKR |

The VH and the VL sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 64), wherein each n is independently 1-4. In some embodiments, n is 1. In some embodiments, n is 2. In some embodiments, n is 3. In some embodiments, n is 4. In some embodiments, the variable regions are not linked with a peptide linker. In some embodiments, the polypeptide comprises SEQ ID NO: 42 and SEQ ID NO: 43. In some embodiments, the targeting moiety comprises a linked peptide represented by a formula of V_{L}-Z-V_{H}, wherein Z is a peptide linker. In some embodiments, the targeting moiety comprises a heavy chain variable region as set forth in SEQ ID NO: 42 linked via a linker sequence GGGGSGGGGSGGGGSGGGGS (SEQ ID NO: 72) to a light chain variable region as set forth in SEQ ID NO: 43. In some embodiments, a targeting moiety comprising a V_{L} linked via a peptide linker to a V_{H} has the sequence as set forth below:

(SEQ ID NO: 44)
DILLTQSPAILSVSPGERVSFSCRASQSIGTSIHWYQQRINDSPRLLIKYASESISG
IPSRFSGSGSGTDFTLSINSVESEDIADYYCQQSNSWPTTFGGGTKLEIKRGGGGSG
GGGSGGGGSGGGGSQVQLQQPGAELVKPGASVKLSCKASGYPFTSYWIHWVKQRPGR
GLEWLGRIDPNSGDTKYNEKFKNKATLTVDKSSTTAYMQLSSLTSEDSAVYYCARSP
YYSNDNSMDYWGQGTSVTVSS

In some embodiments, the targeting moiety comprises a linked peptide represented by a formula of V_{H}-Z-V_{L}, wherein Z is a peptide linker. In some embodiments, the targeting moiety comprises a heavy chain variable region as set forth in SEQ ID NO: 42 linked via a linker sequence GGGGSGGGGSGGGGSGGGGS (SEQ ID NO: 72) to a light chain variable region as set forth in SEQ ID NO: 43. In some embodiments, a targeting moiety comprising a V_{H} linked via a peptide linker to a V_{L} has the sequence as set forth below:

(SEQ ID NO: 45)
QVQLQQPGAELVKPGASVKLSCKASGYPFTSYWIHWVKQRPGRGLEWLGRIDPNSGD
TKYNEKFKNKATLTVDKSSTTAYMQLSSLTSEDSAVYYCARSPYYSNDNSMDYWGQG
TSVTVSSGGGGSGGGGSGGGGSGGGGSDILLTQSPAILSVSPGERVSFSCRASQSIG
TSIHWYQQRINDSPRLLIKYASESISGIPSRFSGSGSGTDFTLSINSVESEDIADYY
CQQSNSWPTTFGGGTKLEIKR

In some embodiments, the targeting moiety (a polypeptide) can bind to CD8.

In some embodiments, the polypeptide binds to CD8. In some embodiments, the polypeptide binds to CD8-alpha. In some embodiments, the polypeptide binds to CD8-beta. In some embodiments, the polypeptide binds to CD8 heterodimer. In some embodiments, the CD8 heterodimer comprises CD8-alpha and CD8-beta subunits. In some embodiments, the polypeptide binds to CD8-alpha homodimer. In some embodiments, the polypeptide that binds to CD8 is an antibody which binds to non-human primate CD8. In some embodiments, the antibody that binds to non-human primate CD8 is an antibody which binds to non-human primate CD8-alpha. In some embodiments, the antibody that binds to non-human primate CD8 is an antibody which binds to non-human primate CD8-beta. In some embodiments, the antibody that binds to non-human primate CD8 is an antibody which binds to non-human primate CD8-alpha homodimer. In some embodiments, the antibody that binds to non-human primate CD8 is an antibody which binds to non-human primate CD8 heterodimer. In some embodiments, the polypeptide that binds to CD8 is an antibody which binds to human CD8. In some embodiments, the antibody that binds to human CD8 is an antibody which binds to human CD8-alpha. In some embodiments, the antibody that binds to human CD8 is an antibody which binds to human CD8-beta. In some embodiments, the antibody that binds to human CD8 is an antibody which binds to human CD8-alpha homodimer. In some embodiments, the antibody that binds to human CD8 is an antibody which binds to human CD8 heterodimer. The sequence of human CD8-alpha (UniProtKB Q8TAW8) is as follows (SEQ ID NO: 46):

(SEQ ID NO: 46)
MALPVTALLLPLALLLHAARPSQFRVSPLDRTWNLGETVELKCQVLLSNPTSGCSWL
FQPRGAAASPTFLLYLSQNKPKAAEGLDTQRFSGKRLGDTFVLILSDFRRENEGCYF
CSALSNSIMYFSHFVPVFLPAKPTTTPAPRPPTPAPTIASQPLSLRPEACRPAAGGA
VHTRGLDFACDIYIWAPLAGTCGVLLLSLVITLYCNHRNRRRVCKCPRPVVKSGDKP
SLSARYV

The sequence of human CD8-beta (UniProtKB Q8TD28) is as follows (SEQ ID NO: 47):

(SEQ ID NO: 47)
MRPRLWLLLAAQLTVLHGNSVLQQTPAYIKVQTNKMVMLSCEAKISLSNMRIYWLRQ
RQAPSSDSHHEFLALWDSAKGTIHGEEVEQEKIAVERDASRFILNLTSVKPEDSGIY
FCMIVGSPELTFGKGTQLSVVDFLPTTAQPTKKSTLKKRVCRLPRPETQKGPLCSPI
TLGLLVAGVLVLLVSLGVAIHLCCRRRRARLREMKQLYK

In some embodiments, the CD8 antibody comprises a Fc region. The Fc region can be linked to the heavy or light chain of the antibody. The Fc region may be fused directly to the heavy or light chain of the antibody or may be fused indirectly to the heavy or light chain of the antibody via, for example, a peptide linker as provided for herein. In some embodiments, the Fc region is an IgG Fc as provided for herein. In some embodiments, the IgG is selected from IgG1, IgG2, IgG3, or IgG4. In some embodiments, the IgG fc is IgG1 Fc as provided for herein. In some embodiments, the antibody comprises an Fc constant region of SEQ ID NO: 26. In some embodiments, the IgG fc is IgG2 Fc as provided for herein. In some embodiments, the antibody comprises an Fc constant region of SEQ ID NO: 27. In some embodiments, the IgG fc is IgG4 Fc as provided for herein. In some embodiments, the antibody comprises an Fc constant region of SEQ ID NO: 28.

In some embodiments, the targeting moiety binds to CD8 and comprises an HCDR1, HCDR2, HCDR3, LCDR1, LCDR2, LCDR3, VH, and VL as recited in Table 3 and Table 4 below:

TABLE 3
CD8 targeting moiety CD8AB1 CDR sequences

| Numbering | | | | | | |
|---|---|---|---|---|---|---|
| System | HCDR1 | HCDR2 | HCDR3 | LCDR1 | LCDR2 | LCDR3 |
| Chothia | RYTFTDY | YPYNGG | DHRYNEGV | RASESVDGF | LASNLES | QQNNEDP |
| | (SEQ ID | (SEQ ID | SFDY | GNSEMN | (SEQ ID | YT (SEQ |
| | NO: 48) | NO: 49) | (SEQ ID | (SEQ ID | NO: 52) | ID NO: |
| | | | NO: 50) | NO: 51) | | 53) |
| Kabat | DYNLH | FIYPYNGGT | SEQ ID | SEQ ID | SEQ ID | SEQ ID |
| | (SEQ ID | GYNQKFKN | NO: 50 | NO: 51 | NO: 52 | NO: 53 |
| | NO: 54) | (SEQ ID | | | | |
| | | NO: 55) | | | | |
| IMGT | RYTFTDY | IYPYNGGT | ARDHRYNE | ESVDGFGNS | LA | SEQ ID |
| | N (SEQ | (SEQ ID | GVSFDY | F (SEQ ID | | NO: 53 |
| | ID NO: | NO: 57) | (SEQ ID | NO: 59) | | |
| | 56) | | NO: 58) | | | |

TABLE 4
CD8 targeting moiety CD8AB1 Variable Regions

| SEQ ID NO: | AB ID NO: | Region | Sequence |
|---|---|---|---|
| 60 | CD8AB1 | VH | EVQLQQSGPELVKPGASVKISCKASRYTFTDYNLHWVKLSHEKSLEWI |
| | | | GFIYPYNGGTGYNQKFKNKAKLTVDYSSSTAYMELRSLTSVDAAVYYC |
| | | | ARDHRYNEGVSFDYWGQGTTLTVSS |
| 61 | CD8AB1 | VL | NIVLTQSPASLAVSLGQRATISCRASESVDGFGNSFMNWYQQKPGQSP |
| | | | KLLIYLASNLESGVPARFSGSGSRTDFTLTIDPVEADDAATYYCQQNN |
| | | | EDPYTFGGGTKLEIKR |

The VH and the VL sequences can be in any format, including, but not limited to an scFv format where the VH and VL regions are linked with a peptide linker. Examples of peptide linkers that can be used to link various peptides provided for herein include, but are not limited to: (GGGGS) n (SEQ ID NO: 64), wherein each n is independently 1-4. In some embodiments, n is 1. In some embodiments, n is 2. In some embodiments, n is 3. In some embodiments, n is 4. In some embodiments, the variable regions are not linked with a peptide linker. In some embodiments, the polypeptide comprises SEQ ID NO: 60 and SEQ ID NO: 61.

In some embodiments, the targeting moiety comprises a linked peptide represented by a formula of V_{L}-Z-V_{H}, wherein Z is a peptide linker. In some embodiments, the targeting moiety comprises a heavy chain variable region as set forth in SEQ ID NO: 60 linked via a linker sequence GGGGSGGGGSGGGGSGGGGS (SEQ ID NO: 72) to a light chain variable region as set forth in SEQ ID NO: 61. In some embodiments, a targeting moiety comprising a V_{L} linked via a peptide linker to a V_{H} has the sequence as set forth below:

(SEQ ID NO: 62)
NIVLTQSPASLAVSLGQRATISCRASESVDGFGNSFMNWYQQKPGQSPKLLIYLASN
LESGVPARFSGSGSRTDFTLTIDPVEADDAATYYCQQNNEDPYTFGGGTKLEIKRGG
GGSGGGGSGGGGSGGGGSEVQLQQSGPELVKPGASVKISCKASRYTFTDYNLHWVKL
SHEKSLEWIGFIYPYNGGTGYNQKFKNKAKLTVDYSSSTAYMELRSLTSVDAAVYYC
ARDHRYNEGVSFDYWGQGTTLTVSS

In some embodiments, the targeting moiety comprises a linked peptide represented by a formula of V_{H}-Z-V_{L}, wherein Z is a peptide linker. In some embodiments, the targeting moiety comprises a heavy chain variable region as set forth in SEQ ID NO: 60 linked via a linker sequence GGGGSGGGGSGGGGSGGGGS (SEQ ID NO: 72) to a light chain variable region as set forth in SEQ ID NO: 61. In some embodiments, a targeting moiety comprising a V_{H} linked via a peptide linker to a V_{L} has the sequence as set forth below:

(SEQ ID NO: 63)
EVQLQQSGPELVKPGASVKISCKASRYTFTDYNLHWVKLSHEKSLEWIGFIYPYNGG
TGYNQKFKNKAKLTVDYSSSTAYMELRSLTSVDAAVYYCARDHRYNEGVSFDYWGQG
TTLTVSSGGGGSGGGGSGGGGSGGGGSNIVLTQSPASLAVSLGQRATISCRASESVD
GFGNSFMNWYQQKPGQSPKLLIYLASNLESGVPARFSGSGSRTDFTLTIDPVEADDA
ATYYCQQNNEDPYTFGGGTKLEIKR

In some embodiments, the targeting moiety comprises an amino acid sequence having at least 70% identity to SEQ ID NO: 44, or is substantially similar to SEQ ID NO: 44, or is an active fragment of SEQ ID NO: 44. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 44. In some embodiments, the targeting moiety comprises an amino acid sequence of SEQ ID NO: 44.

In some embodiments, the targeting moiety comprises an amino acid sequence having at least 70% identity to SEQ ID NO: 45, or is substantially similar to SEQ ID NO: 45, or is an active fragment of SEQ ID NO: 45. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 45. In some embodiments, the targeting moiety comprises an amino acid sequence of SEQ ID NO: 45.

In some embodiments, the targeting moiety comprises an amino acid sequence having at least 70% identity to SEQ ID NO: 62, or is substantially similar to SEQ ID NO: 62, or is an active fragment of SEQ ID NO: 62. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 62. In some embodiments, the targeting moiety comprises an amino acid sequence of SEQ ID NO: 62.

In some embodiments, the targeting moiety comprises an amino acid sequence having at least 70% identity to SEQ ID NO: 63, or is substantially similar to SEQ ID NO: 63, or is an active fragment of SEQ ID NO: 63. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 63. In some embodiments, the targeting moiety comprises an amino acid sequence of SEQ ID NO: 63.

In some embodiments, the targeting moiety as provided for herein is attached to the surface of the virus through a stalk portion, S₁. In some embodiments, the targeting moiety is represented by the formula T-S1, wherein Tis a targeting moiety as provided for herein and S₁ is the stalk portion. In some embodiments, the stalk portion, S1, is as provided in PCT Publication No. WO2024026284, which is hereby incorporated by reference in its entirety. In some embodiments, the stalk portion, S₁, comprises a variant Fc protein as provided for herein and is given by the formula L₁-Fc-L₂-X₁, wherein L₁ is a linker or absent; Fc is the variant Fc protein; L₂ is a linker or absent; and X₁ is a polypeptide comprising a transmembrane domain. Thus, in some embodiments, the formula representing the targeting moiety may also be written T-L₁-Fc-L₂-X₁. In some embodiments, the stalk portion, S₁, does not comprise a variant Fc region and is given by the formula L₃-X₁, wherein L₃ is a flexible polypeptide linker and X₁ is a polypeptide comprising a transmembrane domain. Thus, in some embodiments, the formula representing the targeting moiety may also be written T-L₃-X₁. In some embodiments, the polypeptide comprising a transmembrane domain, X₁, comprises a polypeptide having a formula of ECD-T_{M}-ICD, wherein ECD is an extracellular domain, or a fragment thereof, of a cell surface protein, or is absent, T_{M} is a transmembrane domain of a transmembrane protein, and ICD is an intracellular domain of a protein or is a protein that facilitates the incorporation of the targeting moiety into the envelope of the viral particle, or is absent. Accordingly, the formulas representing the targeting moieties linked to the stalk portions may also be written as T-L₁-Fc-L₂-ECD-T_{M}-ICD or T-L₃-ECD-T_{M}-ICD. Exemplary identities for L₁, L₂, L₃, Fc, ECD, T_{M}, and ICD may be found in PCT Publication No. WO2024026284, which is hereby incorporated by reference in its entirety.

In some embodiments, the targeting moiety comprises a formula of T-L₁-Fc-L₂-ECD-T_{M}-ICD. In some embodiments, T comprises an amino acid sequence having at least 90% identity to SEQ ID NO: 44, SEQ ID NO: 45, SEQ ID NO: 62, or SEQ ID NO: 63. In some embodiments, L₁ is present and comprises the amino acid sequence of SEQ ID NO: 72. In some embodiments, Fc comprises an amino acid sequence having at least 90% identity to SEQ ID NO: 82. In some embodiments, L₂ is absent. In some embodiments, ECD comprises an amino acid sequence having at least 90% identity to SEQ ID NO: 83:

(SEQ ID NO: 83)
KIEVMYPPPYLDNEKSNGTIIHVKGKHLCPSPLFPGPSKP

In some embodiments, T_{M} comprises an amino acid sequence having at least 90% identity to SEQ ID NO: 84:

(SEQ ID NO: 84)
FWVLVVVGGVLACYSLLVTVAFIIFWV

In some embodiments, ICD comprises an amino acid sequence comprising an env incorporation motif, wherein the env incorporation motif comprises an amino acid sequence of SEQ ID NO: 85:

(SEQ ID NO: 85)
NRVRQGYS

In some embodiments, the targeting moiety comprises a formula of T-L₁-Fc-L₂-ECD-T_{M}-ICD. In some embodiments, T comprises an amino acid sequence of SEQ ID NO: 44, SEQ ID NO: 45, SEQ ID NO: 62, or SEQ ID NO: 63. In some embodiments, L₁ is present and comprises the amino acid sequence of SEQ ID NO: 72. In some embodiments, Fc comprises an amino acid sequence of SEQ ID NO: 82. In some embodiments, L₂ is absent. In some embodiments, ECD comprises an amino acid sequence of SEQ ID NO: 83. In some embodiments, T_{M} comprises an amino acid sequence of SEQ ID NO: 84. In some embodiments, ICD comprises an amino acid sequence comprising an env incorporation motif, wherein the env incorporation motif comprises an amino acid sequence of SEQ ID NO: 85.

In some embodiments, the targeting moiety comprises a formula of T-L₁-Fc-L₂-ECD-T_{M}-ICD. In some embodiments, T comprises an amino acid sequence of SEQ ID NO: 44 or SEQ ID NO: 45. In some embodiments, L₁ is present and comprises the amino acid sequence of SEQ ID NO: 72. In some embodiments, Fc comprises an amino acid sequence of SEQ ID NO: 82. In some embodiments, L₂ is absent. In some embodiments, ECD comprises an amino acid sequence of SEQ ID NO: 83. In some embodiments, T_{M} comprises an amino acid sequence of SEQ ID NO: 84. In some embodiments, ICD comprises an amino acid sequence comprising an env incorporation motif, wherein the env incorporation motif comprises an amino acid sequence of SEQ ID NO: 85.

In some embodiments, the targeting moiety comprises a formula of T-L₁-Fc-L₂-ECD-T_{M}-ICD. In some embodiments, T comprises an amino acid sequence of SEQ ID NO: 45. In some embodiments, L₁ is present and comprises the amino acid sequence of SEQ ID NO: 72. In some embodiments, Fc comprises an amino acid sequence of SEQ ID NO: 82. In some embodiments, L₂ is absent. In some embodiments, ECD comprises an amino acid sequence of SEQ ID NO: 83. In some embodiments, T_{M} comprises an amino acid sequence of SEQ ID NO: 84. In some embodiments, ICD comprises an amino acid sequence comprising an env incorporation motif, wherein the env incorporation motif comprises an amino acid sequence of SEQ ID NO: 85.

In some embodiments, the targeting moiety comprises a formula of T-L₃-ECD-T_{M}-ICD. In some embodiments, T comprises an amino acid sequence having at least 90% identity to SEQ ID NO: 44, SEQ ID NO: 45, SEQ ID NO: 62, or SEQ ID NO: 63. In some embodiments, L₃ comprises an amino acid sequence of SEQ ID NO: 64, wherein n is 1, 2, or 4. In some embodiments, ECD comprises an amino acid sequence having at least 90% identity to SEQ ID NO: 86:

(SEQ ID NO: 86)
FVPVFLPAKPTTTPAPRPPTPAPTIASQPLSLRPEACRPAAGGAVHTRGL
DFACD

In some embodiments, T_{M} comprises an amino acid sequence having at least 90% identity to SEQ ID NO: 87:

(SEQ ID NO: 87)
IYIWAPLAGTCGVLLLSLVITLYCNHRN

In some embodiments, ICD comprises an amino acid sequence comprising an env incorporation motif, wherein the env incorporation motif comprises an amino acid sequence of SEQ ID NO: 85 or SEQ ID NO: 88:

(SEQ ID NO: 88)
GGTETSQVAPA

In some embodiments, the targeting moiety comprises a formula of T-L₃-ECD-T_{M}-ICD. In some embodiments, T comprises an amino acid sequence of SEQ ID NO: 44, SEQ ID NO: 45, SEQ ID NO: 62, or SEQ ID NO: 63. In some embodiments, L₃ comprises an amino acid sequence of SEQ ID NO: 64, wherein n is 1, 2, or 4. In some embodiments, ECD comprises an amino acid sequence of SEQ ID NO: 86. In some embodiments, T_{M} comprises an amino acid sequence of SEQ ID NO: 87. In some embodiments, ICD comprises an amino acid sequence comprising an env incorporation motif, wherein the env incorporation motif comprises an amino acid sequence of SEQ ID NO: 85 or SEQ ID NO: 88.

In some embodiments, the targeting moiety comprises a formula of T-L₃-ECD-T_{M}-ICD. In some embodiments, T comprises an amino acid sequence of SEQ ID NO: 44 or SEQ ID NO: 45. In some embodiments, L₃ comprises an amino acid sequence of SEQ ID NO: 64, wherein n is 1, 2, or 4. In some embodiments, ECD comprises an amino acid sequence of SEQ ID NO: 86. In some embodiments, T_{M} comprises an amino acid sequence of SEQ ID NO: 87. In some embodiments, ICD comprises an amino acid sequence comprising an env incorporation motif, wherein the env incorporation motif comprises an amino acid sequence of SEQ ID NO: 85 or SEQ ID NO: 88.

In some embodiments, the targeting moiety comprises a formula of T-L₃-ECD-T_{M}-ICD. In some embodiments, T comprises an amino acid sequence of SEQ ID NO: 45. In some embodiments, L₃ comprises an amino acid sequence of SEQ ID NO: 64, wherein n is 1, 2, or 4. In some embodiments, ECD comprises an amino acid sequence of SEQ ID NO: 86. In some embodiments, T_{M} comprises an amino acid sequence of SEQ ID NO: 87. In some embodiments, ICD comprises an amino acid sequence comprising an env incorporation motif, wherein the env incorporation motif comprises an amino acid sequence of SEQ ID NO: 85 or SEQ ID NO: 88.

In some embodiments, the targeting moiety comprising a formula of T-S₁ comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 89:

(SEQ ID NO: 89)
METDTLLLWVLLLWVPGSTGDSAQVQLQQPGAELVKPGASVKLSCKASGY
PFTSYWIHWVKQRPGRGLEWLGRIDPNSGDTKYNEKFKNKATLTVDKSST
TAYMQLSSLTSEDSAVYYCARSPYYSNDNSMDYWGQGTSVTVSSGGGGSG
GGGSGGGGSGGGGSDILLTQSPAILSVSPGERVSFSCRASQSIGTSIHWY
QQRTNDSPRLLIKYASESISGIPSRFSGSGSGTDFTLSINSVESEDIADY
YCQQSNSWPTTFGGGTKLEIKRASGGGGSGGGGSGGGGSEPKSCDKTHTC
PPCPAPEAAGGPSVFLFPPKPKDTLMASRTPEVTCVVVDVSHEDPEVKFN
WYVDGVEVHNAKTKPREEQYASTYRVVSVLTVLAQDWLNGKEYKCKVSNK
ALGAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCS
VMHEALHNAYTQKSLSLSPGKKIEVMYPPPYLDNEKSNGTIIHVKGKHLC
PSPLFPGPSKPFWVLVVVGGVLACYSLLVTVAFIIFWVRSKRSRLLHSDY
MNRVRQGYS

In some embodiments, the targeting moiety comprising a formula of T-S₁ comprises an amino acid sequence having 90% identity to SEQ ID NO: 89. In some embodiments, the targeting moiety comprising a formula of T-S₁ comprises an amino acid sequence having 95% identity to SEQ ID NO: 89. In some embodiments, the targeting moiety comprising a formula of T-S1 comprises an amino acid sequence having 98% identity to SEQ ID NO: 89. In some embodiments, the targeting moiety comprising a formula of T-S₁ comprises an amino acid sequence of SEQ ID NO: 89.

In some embodiments, the viral vector comprising a mutant VSV-G protein as provided for herein and comprising a targeting moiety as provided for herein further comprises a nucleic acid molecule encoding for a heterologous molecule of interest or “cargo.” For example, heterologous molecule of interest is meant to refer to any product that may be encoded by a nucleic acid molecule. As non-limiting examples, “cargo” or “heterologous molecule of interest” may refer to an siRNA, an shRNA, a peptide, a polypeptide, a protein, a viral payload, a viral genome, or a combination thereof. In some embodiments, the polypeptide is a chimeric antigen receptor (“CAR”).

A “chimeric antigen receptor” or “CAR” as used herein refers to an antigen-binding domain that is fused, directly, or indirectly (e.g. via a hinge or transmembrane domain to an intracellular signaling domain capable of activating or stimulating an immune cell. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, this scFv is fused to a transmembrane domain and then to an intracellular signaling domain. However, the antigen binding domain can be any molecule that can bind to the to target on the cell. For example, the antigen binding domain of a CAR can be an antibody, a scFv antibody, an antigen binding domain, an ankyrin repeat (e.g. DARPIN), a VHH domain antibody, a nanobody, single domain antibody, a FN3 domain, or any combination thereof. In some embodiments, a CAR includes those that solely provide CD3ζ signals upon antigen binding. In some embodiments, the CAR includes those that provide both costimulation (e.g. CD28 or CD137) and activation (CD3 ζ). In some embodiments, the CARs include those that provide multiple costimulation (e.g. CD28 and CD137) and activation (CD3ζ). In various embodiments, the CAR is selected to have high affinity or avidity for the antigen. In some embodiments, the CAR comprises the 4-1BB domain as well. These are merely illustrative in nature and are not limiting to the present embodiments and any chimeric antigen receptor can be delivered in conjunction with the viral particles and vectors provided for herein. These are non-limiting examples of CARs and any CAR construct could be encoded for by the nucleic acid molecule.

In some embodiments, the antigen-binding domain of the CAR comprises a V_{H} domain, a V_{L} domain, or a V_{H} and a V_{L} domain. In some embodiments, the V_{H} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, or any value or range in-between.

(SEQ ID NO: 73)
EVQLVESGGGLVQPGRSLRLSCAASGFTENDYAMHWVRQAPGKGLEWVST
ISWNSGSIGYADSVKGRFTISRDNAKKSLYLQMNSLRAEDTALYYCAKDI
QYGNYYYGMDVWGQGTTVTVSS

In some embodiments, the V_{H} domain comprises an amino acid sequence having at least 90% identity to SEQ ID NO: 73. In some embodiments, the V_{H} domain comprises an amino acid sequence having at least 95% identity to SEQ ID NO: 73. In some embodiments, the V_{H} domain comprises an amino acid sequence having at least 98% identity to SEQ ID NO: 73. In some embodiments, the V_{H} domain comprises an amino acid sequence having at least 99% identity to SEQ ID NO: 73. In some embodiments, the V_{H} domain comprises an amino acid sequence having the sequence of SEQ ID NO: 73.

In some embodiments, the V_{L} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 74, or any value or range in-between.

(SEQ ID NO: 74)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPITFGQ
GTRLEIK

In some embodiments, the V_{L} domain comprises an amino acid sequence having at least 90% identity to SEQ ID NO: 74. In some embodiments, the V_{L} domain comprises an amino acid sequence having at least 95% identity to SEQ ID NO: 74. In some embodiments, the V_{L} domain comprises an amino acid sequence having at least 98% identity to SEQ ID NO: 74. In some embodiments, the V_{L} domain comprises an amino acid sequence having at least 99% identity to SEQ ID NO: 74. In some embodiments, the V_{L} domain comprises an amino acid sequence having the sequence of SEQ ID NO: 74.

In some embodiments, the antigen-binding domain of the CAR comprises a V_{H} domain and a V_{L} domain. In some embodiments, the V_{H} and V_{L} domain are not linked by a linker peptide. In some embodiments, the V_{H} and V_{L} domain are linked by a linker peptide, such as those as provided for herein, including but not limited to: (GGGGS) n (SEQ ID NO: 64), wherein each n is independently 1-5. In some embodiment n is 1. In some embodiment n is 2. In some embodiment n is 3. In some embodiment n is 4. In some embodiment n is 5.

In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 74. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, and comprises a V_{L} having at least 90% identity to SEQ ID NO: 74. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, and comprises a V_{L} having at least 95% identity to SEQ ID NO: 74. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, and comprises a V_{L} having at least 98% identity to SEQ ID NO: 74. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, and comprises a V_{L} having at least 99% identity to SEQ ID NO: 74. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 73, and comprises a V_{L} having the sequence of SEQ ID NO: 74. In some embodiments, the antigen-binding domain of the CAR comprising a V_{H} domain and a V_{L} domain comprises a V_{H} domain having at least 90% identity to SEQ ID NO: 73, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 74. In some embodiments, the antigen-binding domain of the CAR comprising a V_{H} domain and a V_{L} domain comprises a V_{H} domain having at least 95% identity to SEQ ID NO: 73, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 74. In some embodiments, the antigen-binding domain of the CAR comprising a V_{H} domain and a V_{L} domain comprises a V_{H} domain having at least 98% identity to SEQ ID NO: 73, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 74. In some embodiments, the antigen-binding domain of the CAR comprising a V_{H} domain and a V_{L} domain comprises a V_{H} domain having at least 99% identity to SEQ ID NO: 73, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 74. In some embodiments, the antigen-binding domain of the CAR comprising a V_{H} domain and a V_{L} domain comprises a V_{H} domain having an amino acid sequence of SEQ ID NO: 73, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 74. In some embodiments, the antigen-binding domain of the CAR comprising a V_{H} domain and a V_{L} domain comprises a V_{H} domain having at least 90% identity to SEQ ID NO: 73, and comprises a V_{L} having at least 90% identity to SEQ ID NO: 74. In some embodiments, the antigen-binding domain of the CAR comprising a V_{H} domain and a V_{L} domain comprises a V_{H} domain having at least 95% identity to SEQ ID NO: 73, and comprises a V_{L} having at least 90% identity to SEQ ID NO: 74. In some embodiments, the antigen-binding domain of the CAR comprising a V_{H} domain and a V_{L} domain comprises a V_{H} domain having at least 90% identity to SEQ ID NO: 73, and comprises a V_{L} having at least 95% identity to SEQ ID NO: 74. In some embodiments, the antigen-binding domain of the CAR comprising a V_{H} domain and a V_{L} domain comprises a V_{H} domain having at least 95% identity to SEQ ID NO: 73, and comprises a V_{L} having at least 95% identity to SEQ ID NO: 74. In some embodiments, the antigen-binding domain of the CAR comprising a V_{H} domain and a V_{L} domain comprises a V_{H} domain having at least 98% identity to SEQ ID NO: 73, and comprises a V_{L} having at least 98% identity to SEQ ID NO: 74. In some embodiments, the antigen-binding domain of the CAR comprising a V_{H} domain and a V_{L} domain comprises a V_{H} domain having at least 99% identity to SEQ ID NO: 73, and comprises a V_{L} having at least 99% identity to SEQ ID NO: 74. In some embodiments, the antigen-binding domain of the CAR comprising a V_{H} domain and a V_{L} domain comprises a V_{H} domain having an amino acid sequence of SEQ ID NO: 73, and comprises a V_{L} having an amino acid sequence of SEQ ID NO: 74.

In some embodiments, the antigen-binding domain of the CAR comprises a formula of V_{H}-Z-V_{L}, wherein V_{H} is a heavy chain variable region comprising the amino acid sequence of SEQ ID NO: 73, Z is a linker comprising the amino acid sequence GGGGSGGGGSGGGGS (SEQ ID NO: 77), and V_{L} is a light chain variable region comprising the amino acid sequence of SEQ ID NO: 74. In some embodiments, the antigen-binding domain of the CAR comprising a formula of V_{H}-Z-V_{L} has an amino acid sequence as set forth below:

(SEQ ID NO: 75)
EVQLVESGGGLVQPGRSLRLSCAASGFTENDYAMHWVRQAPGKGLEWVST
ISWNSGSIGYADSVKGRFTISRDNAKKSLYLQMNSLRAEDTALYYCAKDI
QYGNYYYGMDVWGQGTTVTVSSGGGGSGGGGSGGGGSEIVLTQSPATLSL
SPGERATLSCRASQSVSSYLAWYQQKPGQAPRLLIYDASNRATGIPARFS
GSGSGTDFTLTISSLEPEDFAVYYCQQRSNWPITFGQGTRLEIK

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 75. In some embodiments, the antigen-binding domain of the CAR comprises an amino acid sequence having at least 90% identity to a sequence of SEQ ID NO: 75. In some embodiments, the antigen-binding domain of the CAR comprises an amino acid sequence having at least 95% identity to a sequence of SEQ ID NO: 75. In some embodiments, the antigen-binding domain of the CAR comprises an amino acid sequence having at least 98% identity to a sequence of SEQ ID NO: 75. In some embodiments, the antigen-binding domain of the CAR comprises an amino acid sequence having at least 99% identity to a sequence of SEQ ID NO: 75. In some embodiments, the antigen-binding domain of the CAR comprises the amino acid sequence of SEQ ID NO: 75.

In some embodiments, the antigen-binding domain of the CAR comprises a formula of V_{L}-Z-V_{H}, wherein V_{L} is a light chain variable region comprising the amino acid sequence of SEQ ID NO: 74, Z is a linker comprising the amino acid sequence GGGGSGGGGSGGGGS (SEQ ID NO: 77), and V_{H} is a light chain variable region comprising the amino acid sequence of SEQ ID NO: 73. In some embodiments, the antigen-binding domain of the CAR comprising a formula of V_{L}-Z-V_{H} has an amino acid sequence as set forth below:

(SEQ ID NO: 76)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPITFGQ
GTRLEIKGGGGSGGGGSGGGGSEVQLVESGGGLVQPGRSLRLSCAASGFT
ENDYAMHWVRQAPGKGLEWVSTISWNSGSIGYADSVKGRFTISRDNAKKS
LYLQMNSLRAEDTALYYCAKDIQYGNYYYGMDVWGQGTTVTVSS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 76. In some embodiments, the antigen-binding domain of the CAR comprises an amino acid sequence having at least 90% identity to a sequence of SEQ ID NO: 76. In some embodiments, the antigen-binding domain of the CAR comprises an amino acid sequence having at least 95% identity to a sequence of SEQ ID NO: 76. In some embodiments, the antigen-binding domain of the CAR comprises an amino acid sequence having at least 98% identity to a sequence of SEQ ID NO: 76. In some embodiments, the antigen-binding domain of the CAR comprises an amino acid sequence having at least 99% identity to a sequence of SEQ ID NO: 76. In some embodiments, the antigen-binding domain of the CAR comprises the amino acid sequence of SEQ ID NO: 76.

In some embodiments, the antigen-binding domain of the CAR comprises a V_{H} domain, a V_{L} domain, or a V_{H} and a V_{L} domain. In some embodiments, the V_{H} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, or any value or range in-between.

(SEQ ID NO: 78)
DIVLTQSPAILSASPGEKVTMICRASSSVNYMDWYQKKPGSSPKPWIYAT
SNLASGVPARFSGSGSGTSYSLTISRVEAEDAATYYCQQWSENPPTFGGG
TKLEIKGSTS

In some embodiments, the V_{H} domain comprises an amino acid sequence having at least 90% identity to SEQ ID NO: 78. In some embodiments, the V_{H} domain comprises an amino acid sequence having at least 95% identity to SEQ ID NO: 78. In some embodiments, the V_{H} domain comprises an amino acid sequence having at least 98% identity to SEQ ID NO: 78. In some embodiments, the V_{H} domain comprises an amino acid sequence having at least 99% identity to SEQ ID NO: 78. In some embodiments, the V_{H} domain comprises an amino acid sequence having the sequence of SEQ ID NO: 78.

In some embodiments, the V_{L} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 79, or any value or range in-between.

(SEQ ID NO: 79)
EVQLQQSGAELVKPGASVKMSCKASGYTFTSYNMHWVKQTPGQGLEWIGA
IYPGNGDTSYNQKFKGKATLTADKSSSTAYMQLSSLTSEDSADYYCARSN
YYGSSYWFFDVWGAGTTVTVSS

In some embodiments, the V_{L} domain comprises an amino acid sequence having at least 90% identity to SEQ ID NO: 79. In some embodiments, the V_{L} domain comprises an amino acid sequence having at least 95% identity to SEQ ID NO: 79. In some embodiments, the V_{L} domain comprises an amino acid sequence having at least 98% identity to SEQ ID NO: 79. In some embodiments, the V_{L} domain comprises an amino acid sequence having at least 99% identity to SEQ ID NO: 79. In some embodiments, the V_{L} domain comprises an amino acid sequence having the sequence of SEQ ID NO: 79.

In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 79. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, and comprises a V_{L} having at least 90% identity to SEQ ID NO: 79. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, and comprises a V_{L} having at least 95% identity to SEQ ID NO: 79. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, and comprises a V_{L} having at least 98% identity to SEQ ID NO: 79. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, and comprises a V_{L} having at least 99% identity to SEQ ID NO: 79. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 78, and comprises a V_{L} having the sequence of SEQ ID NO: 79. In some embodiments, the antigen-binding domain of the CAR comprising a V_{H} domain and a V_{L} domain comprises a V_{H} domain having at least 90% identity to SEQ ID NO: 78, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 79. In some embodiments, the antigen-binding domain of the CAR comprising a V_{H} domain and a V_{L} domain comprises a V_{H} domain having at least 95% identity to SEQ ID NO: 78, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 79. In some embodiments, the antigen-binding domain of the CAR comprising a V_{H} domain and a V_{L} domain comprises a V_{H} domain having at least 98% identity to SEQ ID NO: 78, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 79. In some embodiments, the antigen-binding domain of the CAR comprising a V_{H} domain and a V_{L} domain comprises a V_{H} domain having at least 99% identity to SEQ ID NO: 78, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 79. In some embodiments, the antigen-binding domain of the CAR comprising a Vu domain and a V_{L} domain comprises a V_{H} domain having an amino acid sequence of SEQ ID NO: 78, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 79. In some embodiments, the antigen-binding domain of the CAR comprising a V_{H} domain and a V_{L} domain comprises a V_{H} domain having at least 90% identity to SEQ ID NO: 78, and comprises a V_{L} having at least 90% identity to SEQ ID NO: 79. In some embodiments, the antigen-binding domain of the CAR comprising a V_{H} domain and a V_{L} domain comprises a V_{H} domain having at least 95% identity to SEQ ID NO: 78, and comprises a V_{L} having at least 90% identity to SEQ ID NO: 79. In some embodiments, the antigen-binding domain of the CAR comprising a V_{H} domain and a V_{L} domain comprises a V_{H} domain having at least 90% identity to SEQ ID NO: 78, and comprises a V_{L} having at least 95% identity to SEQ ID NO: 79. In some embodiments, the antigen-binding domain of the CAR comprising a V_{H} domain and a V_{L} domain comprises a V_{H} domain having at least 95% identity to SEQ ID NO: 78, and comprises a V_{L} having at least 95% identity to SEQ ID NO: 79. In some embodiments, the antigen-binding domain of the CAR comprising a V_{H} domain and a V_{L} domain comprises a V_{H} domain having at least 98% identity to SEQ ID NO: 78, and comprises a V_{L} having at least 98% identity to SEQ ID NO: 79. In some embodiments, the antigen-binding domain of the CAR comprising a V_{H} domain and a V_{L} domain comprises a V_{H} domain having at least 99% identity to SEQ ID NO: 78, and comprises a V_{L} having at least 99% identity to SEQ ID NO: 79. In some embodiments, the antigen-binding domain of the CAR comprising a V_{H} domain and a V_{L} domain comprises a V_{H} domain having an amino acid sequence of SEQ ID NO: 78, and comprises a V_{L} having an amino acid sequence of SEQ ID NO: 79.

In some embodiments, the antigen-binding domain of the CAR comprises a formula of V_{H}-Z-V_{L}, wherein V_{H} is a heavy chain variable region comprising the amino acid sequence of SEQ ID NO: 78, Z is a linker comprising the amino acid sequence GGGGSGGGGSGGGGS (SEQ ID NO: 77), and V_{L} is a light chain variable region comprising the amino acid sequence of SEQ ID NO: 79. In some embodiments, the antigen-binding domain of the CAR comprising a formula of V_{H}-Z-V_{L} has an amino acid sequence as set forth below:

(SEQ ID NO: 80)
DIVLTQSPAILSASPGEKVTMICRASSSVNYMDWYQKKPGSSPKPWIYAT
SNLASGVPARFSGSGSGTSYSLTISRVEAEDAATYYCQQWSFNPPTFGGG
TKLEIKGSTSGGGGSGGGGSGGGGSSEVQLQQSGAELVKPGASVKMSCKA
SGYTFTSYNMHWVKQTPGQGLEWIGAIYPGNGDTSYNQKFKGKATLTADK
SSSTAYMQLSSLTSEDSADYYCARSNYYGSSYWFFDVWGAGTTVTVSS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 80. In some embodiments, the antigen-binding domain of the CAR comprises an amino acid sequence having at least 90% identity to a sequence of SEQ ID NO: 80. In some embodiments, the antigen-binding domain of the CAR comprises an amino acid sequence having at least 95% identity to a sequence of SEQ ID NO: 80. In some embodiments, the antigen-binding domain of the CAR comprises an amino acid sequence having at least 98% identity to a sequence of SEQ ID NO: 80. In some embodiments, the antigen-binding domain of the CAR comprises an amino acid sequence having at least 99% identity to a sequence of SEQ ID NO: 80. In some embodiments, the antigen-binding domain of the CAR comprises the amino acid sequence of SEQ ID NO: 80.

In some embodiments, the antigen-binding domain of the CAR comprises a formula of V_{L}-Z-V_{H}, wherein V_{L} is a light chain variable region comprising the amino acid sequence of SEQ ID NO: 79, Z is a linker comprising the amino acid sequence GGGGSGGGGSGGGGS (SEQ ID NO: 77), and V_{H} is a light chain variable region comprising the amino acid sequence of SEQ ID NO: 78. In some embodiments, the antigen-binding domain of the CAR comprising a formula of V_{L}-Z-V_{H} has an amino acid sequence as set forth below:

(SEQ ID NO: 81)
SEVQLQQSGAELVKPGASVKMSCKASGYTFTSYNMHWVKQTPGQGLEWIG
AIYPGNGDTSYNQKFKGKATLTADKSSSTAYMQLSSLTSEDSADYYCARS
NYYGSSYWFFDVWGAGTTVTVSSGGGGSGGGGSGGGGSDIVLTQSPAILS
ASPGEKVTMTCRASSSVNYMDWYQKKPGSSPKPWIYATSNLASGVPARFS
GSGSGTSYSLTISRVEAEDAATYYCQQWSFNPPTFGGGTKLEIKGSTS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 81. In some embodiments, the antigen-binding domain of the CAR comprises an amino acid sequence having at least 90% identity to a sequence of SEQ ID NO: 81. In some embodiments, the antigen-binding domain of the CAR comprises an amino acid sequence having at least 95% identity to a sequence of SEQ ID NO: 81. In some embodiments, the antigen-binding domain of the CAR comprises an amino acid sequence having at least 98% identity to a sequence of SEQ ID NO: 81. In some embodiments, the antigen-binding domain of the CAR comprises an amino acid sequence having at least 99% identity to a sequence of SEQ ID NO: 81. In some embodiments, the antigen-binding domain of the CAR comprises the amino acid sequence of SEQ ID NO: 81.

In some embodiments, the antigen-binding domain of the CAR comprises rituximab, ocrelizumab, obinutuzumab, ofatumumab, ibritumomab tiuxetan, tositumomab, or ublituximab. In some embodiment, the antigen-binding domain comprises rituximab. In some embodiment, the antigen-binding domain comprises ofatumumab. In some embodiments, the CAR comprises the 4-1BB domain as well. These are merely illustrative in nature and are not limiting to the present embodiments and any chimeric antigen receptor can be delivered in conjunction with the viral particles and vectors provided for herein. These are non-limiting examples of CARs and any CAR construct could be encoded for by the nucleic acid molecule.

In some embodiments, the CAR comprises an amino acid sequence that is at least 85%, 86%, 87%, 88%, 89%, 90%, 91%, 92%, 93%, 94%, 95%, 96%, 97%, 98%, 99%, or 100% identical to a sequence of SEQ ID NO: 90:

(SEQ ID NO: 90)
MALPVTALLLPLALLLHAARPGSEVQLVESGGGLVQPGRSLRLSCAASGE
TFNDYAMHWVRQAPGKGLEWVSTISWNSGSIGYADSVKGRFTISRDNAKK
SLYLQMNSLRAEDTALYYCAKDIQYGNYYYGMDVWGQGTTVTVSSGGGGS
GGGGSGGGGSEIVLTQSPATLSLSPGERATLSCRASQSVSSYLAWYQQKP
GQAPRLLIYDASNRATGIPARFSGSGSGTDFTLTISSLEPEDFAVYYCQQ
RSNWPITFGQGTRLEIKSGLDFVPVFLPAKPTTTPAPRPPTPAPTIASQP
LSLRPEACRPAAGGAVHTRGLDFACDIYIWAPLAGTCGVLLLSLVITLYC
NHRNKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR

or is substantially similar to SEQ ID NO: 90, or is an active fragment of SEQ ID NO: 90. In some embodiments, the CAR comprises an amino acid sequence that is at least 90% identical to a sequence of SEQ ID NO: 90. In some embodiments, the CAR comprises an amino acid sequence that is at least 95% identical to a sequence of SEQ ID NO: 90. In some embodiments, the CAR comprises an amino acid sequence that is at least 98% identical to a sequence of SEQ ID NO: 90. In some embodiments, the CAR comprises an amino acid sequence of SEQ ID NO: 90. In some embodiments, the sub domains of the CAR (e.g., the antigen binding domain, hinge domain, transmembrane domain, costimulatory domain, signaling domain) are as provided in PCT Publication No. WO2024026284, which is hereby incorporated by reference in its entirety.

In some embodiments, the pseudotyped viral vector further comprises a heterologous nucleic acid molecule encoding a cargo of interest. The nucleic acid molecule may be useful for modulating the expression of a target gene. In some embodiments, the cargo can be used to modulate the activity of a cell or express a protein that is trafficked to the surface of the target cell. Therefore, in some embodiments, the nucleic acid may comprise an siRNA or an shRNA. The nucleic acid may also encode for a cargo of interest. Therefore, in some embodiments, the cargo of interest may comprise a polypeptide or portion thereof, a protein or portion thereof, a chimeric antigen receptor or portion thereof, or a tumor antigen or a portion thereof. In some embodiments, the cargo of interest is an antibody that is produced by the virus, which can then be secreted by the cell that is infected with the virus. The term “protein” can refer to any polypeptide that carries a native function in a cellular environment. Therefore, in some embodiments, the protein encoded by the nucleic acid cargo of interest may comprise an enzyme, a nuclear receptor, a transporter, a ribosomal protein, a membrane bound protein, a cytoplasmic protein, a G-protein coupled receptor, a voltage gated ion channel, a secretory protein, a mitochondria protein, a cytokine, a chimeric antigen receptor, a tumor antigen, or a portion or chimeric species thereof.

Without being bound to any particular theory, the viral vector comprising the mutant VSV-G protein as provided for herein that comprises a targeting moiety can be used to express the heterologous molecule of interest in the target cell. Thus, for example, the CAR can be expressed in a T cell that is targeted by a viral vector pseudotyped with a VSV-G protein as provided for herein. Where the T cell is the intended target the viral vector can comprise a targeting moiety that binds to a target on the surface of a T cell, such as, but not limited to CD2, CD3, CD4, CD5, CD7 or CD8. In some embodiments, the target is CD2. In some embodiments, the target is CD3. In some embodiments, the target is CD4. In some embodiments, the target is CD5. In some embodiments, the target is CD6. In some embodiments, the target is CD7. In some embodiments, the target is CD8.

In some embodiments, the pseudotyped viral vector is a recombinant lentivirus. In some embodiments, the recombinant pseudotyped viral vector is replication competent. In some embodiments, the recombinant pseudotyped viral vector is replication incompetent.

### Exemplary Viral Vectors:

In some embodiments, a viral vector is provided, the viral vector comprising a heterologous viral glycoprotein and a targeting moiety, wherein the heterologous viral glycoprotein comprises an amino acid sequence selected from SEQ ID NO: 22, SEQ ID NO: 23, SEQ ID NO: 24, or SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a viral vector is provided, the viral vector comprising a heterologous viral glycoprotein and a targeting moiety, wherein the heterologous viral glycoprotein comprises an amino acid sequence selected from SEQ ID NO: 22 or SEQ ID NO: 23, and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a viral vector is provided, the viral vector comprising a heterologous viral glycoprotein and a targeting moiety, wherein the heterologous viral glycoprotein comprises an amino acid sequence of SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a viral vector is provided, the viral vector comprising a heterologous viral glycoprotein and a targeting moiety, wherein the heterologous viral glycoprotein comprises an amino acid sequence of SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

### Exemplary Pharmaceutical Compositions:

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence selected from SEQ ID NO: 22, SEQ ID NO: 23, SEQ ID NO: 24, or SEQ ID NO: 25 and wherein the targeting moiety comprises an amino acid sequence of SEQ ID NO: 89. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence selected from SEQ ID NO: 22, SEQ ID NO: 23, SEQ ID NO: 24, or SEQ ID NO: 25 and wherein the targeting moiety comprises an amino acid sequence of SEQ ID NO: 89. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence selected from SEQ ID NO: 22, SEQ ID NO: 23, SEQ ID NO: 24, or SEQ ID NO: 25 and wherein the targeting moiety comprises an amino acid sequence of SEQ ID NO: 89. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence selected from SEQ ID NO: 22, SEQ ID NO: 23, SEQ ID NO: 24, or SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence of SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about human serum albumin in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence selected from SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence of SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence selected from SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 24 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 24 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence of SEQ ID NO: 24 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence selected from SEQ ID NO: 24 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein and a targeting moiety, wherein the heterologous viral glycoprotein comprises an amino acid sequence of SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence of SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein and a targeting moiety, wherein the heterologous viral glycoprotein comprises an amino acid sequence selected from SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 20 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 14 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 22 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 20 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 14 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 23 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 20 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 24 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 14 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 24 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 20 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. At least 99% identity to SEQ ID NO: 90, or at least 100% identity to SEQ ID NO: 90.

In some embodiments, a pharmaceutical composition is provided, the pharmaceutical composition comprising a viral vector; a L-Histidine buffer at a concentration of about 14 mM; sodium chloride at a concentration of about 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein and a targeting moiety, wherein the heterologous viral glycoprotein comprises an amino acid sequence selected from SEQ ID NO: 25 and wherein the targeting moiety comprises an amino acid sequence of SEQ ID NO: 89. In some embodiments, the viral vector further comprises a nucleic acid molecule encoding a heterologous molecule of interest. In some embodiments, the heterologous molecule of interest is as provided for herein. In some embodiments, the heterologous molecule of interest is a CAR as provided for herein. In some embodiments, the CAR comprises an amino acid sequence having at least 85% identity to SEQ ID NO: 90, at least 90% identity to SEQ ID NO: 90, at least 95% identity to SEQ ID NO: 90. at least 99% identity to SEQ ID NO: 90, or at least 100% identity to SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence selected from SEQ ID NO: 22, SEQ ID NO: 23, SEQ ID NO: 24, or SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence selected from SEQ ID NO: 22, SEQ ID NO: 23, SEQ ID NO: 24, or SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence selected from SEQ ID NO: 22, SEQ ID NO: 23, SEQ ID NO: 24, or SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about human serum albumin in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence selected from SEQ ID NO: 22, SEQ ID NO: 23, SEQ ID NO: 24, or SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 22, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 22, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 22, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 22, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 23, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 23, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 23, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 23, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 24, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 24, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 24, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about human serum albumin in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 24, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a pharmaceutically acceptable buffer at a concentration of about 5 mM to about 100 mM; a pharmaceutically acceptable salt at a concentration of about 100 mM to about 800 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 5 mM to about 50 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.0 to about 8.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a pharmaceutically acceptable buffer at a concentration of about 10 mM to about 30 mM; a pharmaceutically acceptable salt at a concentration of about 100 mM to about 400 mM; a sugar in an amount of about 1% (w/v) to about 5% (w/v); and a protein excipient in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90. In some embodiments, the pharmaceutically acceptable buffer is as provided for herein. In some embodiments, the pharmaceutically acceptable salt is as provided for herein. In some embodiments, the sugar is as provided for herein. In some embodiments, the protein excipient is as provided for herein.

In some embodiments, a pharmaceutical composition is provided, the pharmaceutical composition comprising a viral vector; a L-Histidine buffer at a concentration of about 10 mM to about 30 mM; sodium chloride at a concentration of about 100 mM to about 400 mM; sucrose in an amount of about 1% (w/v) to about 5% (w/v); and non-recombinant human serum albumin in an amount of about 0.1% (w/v) to about 3% (w/v), wherein the pharmaceutical composition has a pH of about 6.5 to about 7.5, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 20 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 22, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 14 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 22, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 20 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 23, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 14 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 23, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 20 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 24, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 14 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 24, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 20 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90.

In some embodiments, a pharmaceutical composition is provided, the pharmaceutical composition comprising a viral vector; a L-Histidine buffer at a concentration of 14 mM; sodium chloride at a concentration of 150 mM; sucrose in an amount of 4% (w/v); and non-recombinant human serum albumin in an amount of 0.5% (w/v), wherein the pharmaceutical composition has a pH of about 7.0, wherein the viral vector comprises a heterologous viral glycoprotein, a targeting moiety, and a nucleic acid molecule encoding a heterologous molecule of interest, wherein the heterologous viral glycoprotein comprises an amino acid sequence of SEQ ID NO: 25, wherein the targeting moiety comprises an amino acid sequence of SEQ ID NO: 89, and wherein the heterologous molecule of interest comprises a CAR comprising an amino acid sequence of SEQ ID NO: 90.

### Methods

In some embodiments, methods of delivering a cargo of interest to a cell are provided. In some embodiments, the methods comprise contacting the cell with the pharmaceutical compositions as provided for herein.

In some embodiments, methods of delivering a cargo of interest to a cell in a subject are provided. In some embodiments, the methods comprise administering to the subject the pharmaceutical compositions as provided for herein. In some embodiments, the cargo is a chimeric antigen receptor or as otherwise provided for herein.

In some embodiments, methods for of delivering a chimeric antigen receptor to a T-cell in a subject are provided. In some embodiments, the methods comprising administering to the subject the pharmaceutical compositions as provided for herein, wherein the pseudotyped viral-like particle or viral vector of the pharmaceutical compositions comprise a heterologous nucleic acid molecule encoding the chimeric antigen receptor.

Methods of making the viruses, viral like particles, or viral vectors comprising a mutant VSV-G protein are also provided. In some embodiments, the methods comprise transfecting or transducing a packaging cell line with the nucleic acid molecules encoding a mutant VSV-G protein as provided for herein under conditions sufficient to produce the pseudotyped viral-like particles or viral vectors. In some embodiments, the methods comprise transfecting or transducing a packaging cell line with the plurality of nucleic acid molecules provided for herein under conditions sufficient to produce the pseudotyped viral-like particles or viral vectors. In some embodiments, methods further comprise isolating the pseudotyped viral-like particle or viral vector. In some embodiments, the nucleic acid molecules also comprise a nucleic acid molecule encoding a targeting moiety and/or a cargo that is to be delivered by the viral vector that is produced. In some embodiments, the method of making the viral like particles or vectors of the present disclosure are as provided in International Patent Application No. PCT/US2024/014002, which is hereby incorporated by reference in its entirety.

### Methods of Treating Cancer

Also provided for herein are methods of treating cancer in a subject. In some embodiments, the methods comprise administering to the subject a pharmaceutical composition as provided for herein, wherein the lentiviral vector of the pharmaceutical composition comprises a heterologous nucleic acid molecule encoding a chimeric antigen receptor as provided for herein.

In some embodiments, the methods comprise administering to the subject immune cells that have been transduced by the pseudotyped viral-like particle or viral vectors as provided for herein. In some embodiments, the immune cells are first isolated from the subject prior to transduction by the pseudotyped viral-like particle or viral vectors as provided for herein.

Also provided herein are methods of treating a disease in a subject in need thereof.

In some embodiments, the methods provided include, but are not limited to, methods of treating a disease in a subject in need thereof, comprising administering to the subject a pharmaceutical composition as provided for herein, wherein the lentiviral vector of the pharmaceutical composition comprises a heterologous nucleic acid molecule encoding a chimeric antigen receptor as provided for herein.

In certain embodiments, the disease is a cancer. In addition, the compositions provided for herein can be used in methods for the treatment of any condition related to a cancer, such as a cell-mediated immune response against a tumor cell(s), where it is desirable to treat or alleviate the disease. The types of cancers to be treated include, but are not limited to, carcinoma, blastoma, sarcoma, certain leukemia or lymphoid malignancies, benign and malignant tumors, malignancies e.g., sarcomas, carcinomas, and melanomas. Other exemplary cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by the methods disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

In certain exemplary embodiments, the compositions provided herein can be used in methods to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In some embodiments, methods of treating multiple myeloma are provided. In some embodiments, the multiple myeloma is refractory myeloma. In some embodiments, the multiple myeloma is relapsed myeloma.

In certain exemplary embodiments, the in vivo modified immune cells produced using the compositions provided herein are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In some embodiments, the melanoma is cutaneous melanoma In some embodiments, the melanoma is refractory melanoma. In some embodiments, the melanoma is relapsed melanoma.

In some embodiments, the compositions provided herein are used to treat a sarcoma, or a condition related to sarcoma. Examples of sarcoma or conditions related thereto include, without limitation, angiosarcoma, chondrosarcoma, chordoma, endotheliosarcoma, Ewing's sarcoma, fibrosarcoma, gastrointestinal stromal tumor, leiomyosarcoma, liposarcoma, lymphangiosarcoma, lymphangioendotheliosarcoma, mesothelioma, malignant peripheral nerve sheath tumor, myxosarcoma, osteogenic sarcoma, osteosarcoma, pleomorphic sarcoma, rhabdomyosarcoma, synovioma, synovial sarcoma, and other soft tissue sarcomas. In some embodiments, the sarcoma is synovial sarcoma. In some embodiments, the sarcoma is liposarcoma such as myxoid/round cell liposarcoma, differentiated/dedifferentiated liposarcoma, or pleomorphic liposarcoma. In some embodiments, the sarcoma is myxoid/round cell liposarcoma. In some embodiments, the sarcoma is refractory sarcoma. In some embodiments, the sarcoma is relapsed sarcoma.

In some embodiments, the subject has been treated with a therapeutic agent targeting the disease or condition, e.g. the tumor, prior to administration of the composition. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the composition is administered prophylactically, e.g., to reduce the likelihood of or prevent relapse. In some aspects, the subject has not received prior treatment with another therapeutic agent.

The administration of the compositions may be carried out in any convenient manner known to those of skill in the art. For example, the compositions may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, intraperitoneally, intranasally, intracranially, or intraosseously. In other instances, the compositions is injected directly into a site of a local disease site in the subject, a lymph node, an organ, a tumor, and the like.

For the prevention or treatment of disease, the appropriate dosage may depend on the type of disease to be treated, the severity and course of the disease, whether the composition is administered for preventive or therapeutic purposes, previous therapy, the subject's clinical history and response to the treatment, and the discretion of the attending physician. The composition is, in some embodiments, suitably administered to the subject at one time or over a series of treatments.

In some embodiments, the composition is administered as part of a combination treatment, such as simultaneously with or sequentially with, in any order, another therapeutic intervention, such as an antibody or produced cell or receptor or agent, such as a cytotoxic or therapeutic agent. The composition(s), in some embodiments, is co-administered with one or more additional therapeutic agents or in connection with another therapeutic intervention, either simultaneously or sequentially in any order. In some contexts, the composition is co-administered with another therapy sufficiently close in time such that the composition enhances the effect of one or more additional therapeutic agents, or vice versa. In some embodiments, the composition is administered prior to the one or more additional therapeutic agents. In some embodiments, the composition is administered after the one or more additional therapeutic agents. In some embodiments, the one or more additional agents includes a cytokine, such as IL-2, for example, to enhance persistence. In some embodiments, the methods comprise administration of a chemotherapeutic agent. In some embodiments, the methods do not comprise the administration of a chemotherapeutic agent.

In certain embodiments, the compositions may be administered to a subject in combination with an immune checkpoint antibody (e.g., an anti-PD1, anti-CTLA-4, or anti-PDL1 antibody). For example, viral vectors may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVO®) or an antigen-binding fragment thereof. In certain embodiments, the compositions may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the composition may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the viral vector. In certain embodiments, combination treatment comprising an immune checkpoint modulator may increase the therapeutic efficacy of a therapy comprising a composition as provided herein. The other therapeutic can be administered simultaneously, before, or after the compositions provided herein are administered to the subject.

In certain embodiments, the subject is provided a secondary treatment. Secondary treatments include but are not limited to chemotherapy, radiation, surgery, and medications. In some embodiments, the subject is not provided a secondary treatment.

In some embodiments, the methods are performed without a lymphodepletion step, such as the administration of cyclophosphamide and/or fludarabine.

In some embodiments, the subject can be administered a conditioning therapy after the administration of the compositions to kill certain immune cells that are not transduced with the CAR encoded by the compositions. This can be done by including a selection marker that is encoded by the nucleic acid cargo of interest. In some embodiments, the conditioning therapy comprises administering an effective amount of cyclophosphamide to the subject. In some embodiments, the conditioning therapy comprises administering an effective amount of fludarabine to the subject. In some embodiments, the conditioning therapy comprises administering an effective amount of a combination of cyclophosphamide and fludarabine to the subject.

In some embodiments, a specific dosage regimen of the present disclosure includes a lymphodepletion step after the administration of the composition. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

It is known in the art that one of the adverse effects of the use of CAR T cells can be the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, in some embodiments, the methods comprise, following the diagnosis of CRS, appropriate CRS management strategies to mitigate the physiological symptoms of uncontrolled inflammation without dampening the antitumor efficacy of the in vivo generated cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6 (6): 664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In some embodiments, methods of treating cancer in a subject in need thereof are provided, the methods comprising administering to the subject any of the compositions, such as the viral particle(s), provided herein.

The embodiments provided for herein can be used for many purposes, since the a lentiviral vectors of the pharmaceutical compositions provided for herein are capable of fusing with a target cell and can be used to deliver a gene or other heterologous sequence of interest.

### Enumerated Embodiments

The following examples are illustrative, but not limiting, of the compounds, compositions and methods described herein. Other suitable modifications and adaptations known to those skilled in the art are within the scope of the following embodiments.

1. A pharmaceutical composition comprising:
(i) a lentiviral vector;
(ii) a pharmaceutically acceptable buffer at a concentration of about 5 mM to about 100 mM;
(iii) a pharmaceutically acceptable salt at a concentration of about 100 mM to about 800 mM;
(iv) a sugar in an amount of about 1% (w/v) to about 5% (w/v); and
(v) a protein excipient in an amount of about 0.1% (w/v) to about 3% (w/v), wherein the pharmaceutical composition has a pH of about 6.0 to about 8.0.

2. The pharmaceutical composition of embodiment 1, wherein the lentiviral vector is present at a concentration of about 1.0E⁺⁰⁵ to about 1.0E⁺⁰⁹ TUs/mL.

3. The pharmaceutical composition of any one of embodiments 1-2, wherein the lentiviral vector is present at a concentration of about 1.0E⁺⁰⁵ to about 1.0E⁺⁰⁹ TUs/mL, about 1.0E⁺⁰⁵ to about 1.0E⁺⁰⁸ TUs/mL, about 1.0E⁺⁰⁵ to about 1.0E⁺⁰⁷ TUs/mL, about 1.0E⁺⁰⁵ to about 1.0E⁺⁰⁶ TUs/mL, about 1.0E⁺⁰⁶ to about 1.0E⁺⁰⁹ TUs/mL, about 1.0E⁺⁰⁶ to about 1.0E⁺⁰⁸ TUs/mL, about 1.0E⁺⁰⁶ to about 1.0E⁺⁰⁷ TUs/mL, about 1.0E⁺⁰⁷ to about 1.0E⁺⁰⁹ TUs/mL, about 1.0E⁺⁰⁷ to about 1.0E⁺⁰⁸ TUs/mL, or about 1.0E⁺⁰⁸ to about 1.0E⁺⁰⁹ TUs/mL.

4. The pharmaceutical composition of any one of embodiments 1-3, wherein the pharmaceutically acceptable buffer is present at a concentration of about 5 mM to about 100 mM, about 5 mM to about 70 mM, about 5 mM to about 60 mM, about 10 mM to about 60 mM, about 10 mM to about 50 mM, about 10 mM to about 40 mM, about 15 mM to about 30 mM, or about 15 mM to about 25 mM.

5. The pharmaceutical composition of any one of embodiments 1-4, wherein the pharmaceutically acceptable buffer is histidine buffer, HEPES, phosphoric acid buffer, citric acid buffer, acetic acid buffer, succinic acid buffer, phosphate buffer, acetate buffer, citrate buffer, succinate buffer, ascorbic acid buffer, glutamic acid buffer, lactic acid buffer, maleic acid buffer, trometamol buffer, or gluconic acid buffer, or any combination thereof.

6. The pharmaceutical composition of any one of embodiments 1-5, wherein the pharmaceutically acceptable buffer is a histidine buffer.

7. The pharmaceutical composition of embodiment 6, wherein the histidine buffer is present at a concentration of about 10 mM to about 30 mM.

8. The pharmaceutical composition of any one of embodiments 6-7, wherein the histidine buffer is present at a concentration of about 20 mM.

9. The pharmaceutical composition of embodiment 1-8, wherein the pharmaceutical composition has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

10. The pharmaceutical composition of any one of embodiments 1-9, wherein the pharmaceutical composition has a pH of about 6.5 to about 7.5.

11. The pharmaceutical composition of any one of embodiments 1-10, wherein the pharmaceutical composition has a pH of about 7.0.

12. The pharmaceutical composition of any one of embodiments 1-11, wherein the pharmaceutically acceptable buffer is a histidine buffer and the pharmaceutical composition has a pH of about 7.0.

13. The pharmaceutical composition of any one of embodiments 1-12, wherein the pharmaceutically acceptable salt is sodium chloride, potassium chloride, magnesium chloride, or calcium chloride, or any combination thereof.

14. The pharmaceutical composition of any one of embodiments 1-13, wherein the pharmaceutically acceptable salt is present at a concentration of about 100 mM to about 800 mM, about 100 mM to about 500 mM, about 100 mM to about 400 mM, about 100 mM to about 250 mM, about 100 mM to about 200 mM, about 100 mM to about 175 mM, about 100 mM to about 150 mM or about 125 mM to about 150 mM.

15. The pharmaceutical composition of any one of embodiments 1-14, wherein the pharmaceutically acceptable salt is present at a concentration of about 100 mM, about 110 mM, about 120 mM, about 130 mM, about 140 mM, about 150 mM, about 160 mM, about 170 mM, about 180 mM, about 190 mM, about 200 mM, about 250 mM, about 400 mM, about 500 mM or about 800 mM

16. The pharmaceutical composition of any one of embodiments 1-14, wherein the pharmaceutically acceptable salt is sodium chloride.

17. The pharmaceutical composition of embodiment 16, wherein the sodium chloride is present at a concentration of about 100 mM to about 800 mM, about 100 mM to about 500 mM, about 100 mM to about 400 mM, about 100 mM to about 250 mM, about 100 mM to about 200 mM, about 100 mM to about 175 mM, about 100 mM to about 150 mM, or about 125 mM to about 150 mM.

18. The pharmaceutical composition of any one of embodiments 16-17, wherein the sodium chloride is present at a concentration of about 100 mM, about 110 mM, about 120 mM, about 130 mM, about 140 mM, about 150 mM, about 160 mM, about 170 mM, about 180 mM, about 190 mM, about 200 mM, about 250 mM, about 400 mM, about 500 mM, or about 800 mM

19. The pharmaceutical composition of any one of embodiments 16-18, wherein the sodium chloride is present at a concentration of about 150 mM.

20. The pharmaceutical composition of any one of embodiments 1-19, wherein the sugar is sucrose or trehalose, or a combination thereof.

21. The pharmaceutical composition of any one of embodiments 1-20, wherein the sugar is present at an amount of about 1% (w/v) to about 10% (w/v), about 1% (w/v) to about 5% (w/v), or about 2% (w/v) to about 4% (w/v).

22. The pharmaceutical composition of any one of embodiments 1-21, wherein the sugar is present in amount of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

23. The pharmaceutical composition of any one of embodiments 1-22, wherein the sugar is sucrose.

24. The pharmaceutical composition of embodiment 23, wherein the sucrose is present at about 1% (w/v) to about 10% (w/v), about 1% (w/v) to about 5% (w/v), or about 2% (w/v) to about 4% (w/v).

25. The pharmaceutical composition of any one of embodiments 23-24, wherein the sucrose is present at about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

26. The pharmaceutical composition of any one of embodiments 23-25, wherein the sucrose is present at about 4% (w/v).

27. The pharmaceutical composition of any one of embodiments 1-26, wherein the pharmaceutically acceptable salt is sodium chloride and the sugar is sucrose.

28. The pharmaceutical composition of any one of embodiments 1-27, wherein the protein excipient is recombinant human serum albumin, non-recombinant human serum albumin, or a combination thereof.

29. The pharmaceutical composition of any one of embodiments 1-28, wherein the protein excipient is present at an amount of about 0.1% (w/v) to about 3% (w/v).

30. The pharmaceutical composition of any one of embodiments 1-29, wherein the protein excipient is present at an amount of about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3.0% (w/v).

31. The pharmaceutical composition of any one of embodiments 1-30, wherein the protein excipient is recombinant human serum albumin.

32. The pharmaceutical composition of embodiment 31, wherein the recombinant human serum albumin is present at an amount of about 0.1% (w/v) to about 3% (w/v).

33. The pharmaceutical composition of embodiment 31, wherein the protein excipient is a plant recombinant human serum albumin, such as Exbumin®.

34. The pharmaceutical composition of embodiments 33, wherein the plant recombinant human serum albumin is present at an amount of about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3.0% (w/v).

35. The pharmaceutical composition of any one of embodiments 33-34, wherein the plant recombinant human serum albumin is present at an amount of about 0.5% (w/v).

36. The pharmaceutical composition of any one of embodiments 1-30, wherein the protein excipient is non-recombinant human serum albumin.

37. The pharmaceutical composition of embodiment 36, wherein the non-recombinant human serum albumin is present at an amount of about 0.1% (w/v) to about 3% (w/v).

38. The pharmaceutical composition of embodiment 36 or 37, wherein the non-recombinant human serum albumin is USP grade non-recombinant human serum albumin.

39. The pharmaceutical composition of any one of embodiments 36-38, wherein the non-recombinant human serum albumin is present at an amount of about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3.0% (w/v).

40. The pharmaceutical composition of any one of embodiments 36-39, wherein the non-recombinant human serum albumin is present at an amount of about 0.5% (w/v).

41. The pharmaceutical composition of any one of embodiments 1-40, wherein the lentiviral vector is a VSV-G pseudotyped lentivirus.

42. The pharmaceutical composition of embodiment 41, wherein the VSV-G pseudotyped lentivirus comprises a VSV-G polypeptide comprising a mutation that corresponds to a mutation at position 182 of SEQ ID NO: 2.

43. A pharmaceutical composition comprising:
(i) a VSV-G pseudotyped lentivirus
(ii) about 10 mM to about 30 mM of a L-Histidine buffer;
(iii) about 100 mM to about 400 mM of sodium chloride;
(iv) about 1% (w/v) to about 5% (w/v) of sucrose; and
(v) about 0.1% (w/v) to about 2% (w/v) of a non-recombinant human serum albumin,
wherein the pharmaceutical composition has a pH of about 5.5 to about 8.0.

44. The pharmaceutical composition of embodiment 43, wherein the VSV-G pseudotyped lentivirus is present at a concentration of about 1.0E⁺⁰⁵ to 1.0E⁺⁰⁹ TUs/mL.

45. The pharmaceutical composition of any one of embodiments 43-44, wherein the L-Histidine is present at a concentration of about 15-25 mM and the pharmaceutical composition has a pH of about 6 to about 7.

46. The pharmaceutical composition of any one of embodiments 43-45, wherein the L-Histidine is present at a concentration of about 20 mM and the pharmaceutical composition has a pH of about 7.0.

47. The pharmaceutical composition of any one of embodiments 43-46, wherein sodium chloride is present at a concentration of about 125 mM to about 175 mM.

48. The pharmaceutical composition of embodiment 47, wherein the sodium chloride is present at a concentration of about 150 mM.

49. The pharmaceutical composition of any one of embodiments 43-48, wherein sucrose is present in an amount of about 2% (w/v) to about 5% (w/v).

50. The pharmaceutical composition of embodiment 49, wherein sucrose is present in an amount of about 4% (w/v).

51. The pharmaceutical composition of any one of embodiments 43-50, wherein the non-recombinant human serum albumin is present at about 0.1% (w/v) to about 1.5% (w/v).

52. The pharmaceutical composition of embodiment 51, wherein the non-recombinant human serum albumin is present at about 0.5% (w/v).

53. The pharmaceutical composition of any one of embodiments 43-52, wherein the non-recombinant human serum albumin is USP grade non-recombinant human serum albumin.

54. The pharmaceutical composition of any one of embodiments 43-53, wherein the VSV-G pseudotyped lentivirus comprises a VSV-G polypeptide comprising a mutation that corresponds to a mutation at position 182 of SEQ ID NO: 2.

55. The pharmaceutical composition of embodiment 54, wherein the VSV-G polypeptide comprises an amino acid sequence of SEQ ID NO: 2 with a mutation at position 182 and at least 70% identity to SEQ ID NO: 2.

56. The pharmaceutical composition of any one of embodiments 54-55, wherein the VSV-G polypeptide comprises a I182A, I182D, or I182E mutation as compared to SEQ ID NO: 2.

57. The pharmaceutical composition of any one of embodiments 54-56 wherein the VSV-G polypeptide comprises the sequence of SEQ ID NO: 1 with a with a mutation at position 198 of SEQ ID NO: 1 and at least 70% identity to SEQ ID NO: 2.

58. The pharmaceutical composition of any one of embodiments 54-57, wherein the VSV-G polypeptide comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to a sequence of SEQ ID NO: 4.

59. The pharmaceutical composition of any one of embodiments 54-58, wherein the VSV-G polypeptide comprises a sequence of SEQ ID NO: 4.

60. The pharmaceutical composition of any one of embodiments 54-59, wherein the VSV-G polypeptide comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to a sequence of SEQ ID NO: 5.

61. The pharmaceutical composition of any one of embodiments 54-60, wherein the VSV-G polypeptide comprises a sequence of SEQ ID NO: 5.

62. The pharmaceutical composition of embodiments 54-61, wherein the VSV-G polypeptide further comprises a mutation in the VSV-G protein that corresponds to a mutation as described in US 2020/0216502.

63. The pharmaceutical composition of any one of embodiments 54-62, wherein the VSV-G polypeptide further comprises a mutation in the VSV-G protein that corresponds to a position of 8, 10, 47, 209 and/or 354 as compared to SEQ ID NO: 2.

64. The pharmaceutical composition of any one of embodiments 54-63, wherein the VSV-G polypeptide further comprises a mutation that corresponds to a position 8 in SEQ ID NO: 2, wherein the mutation any amino acid different from the amino acid indicated at that position in the sequence SEQ ID NO: 2, except Y.

65. The pharmaceutical composition of any one of embodiments 54-64, wherein the VSV-G polypeptide further comprises a mutation that corresponds to a position 209 in SEQ ID NO: 2, wherein the mutation any amino acid different from the amino acid indicated at that position in the sequence SEQ ID NO: 2, except H.

66. The pharmaceutical composition of any one of embodiments 54-65, wherein the VSV-G polypeptide further comprises a mutation that corresponds to a position 47 in SEQ ID NO: 2, wherein the mutation any amino acid different from the amino acid indicated at that position in the sequence SEQ ID NO: 2, except K or R.

67. The pharmaceutical composition of any one of embodiments 54-66, wherein the VSV-G polypeptide further comprises a mutation that corresponds to position 354 in SEQ ID NO: 2, wherein the mutation is any amino acid different from the amino acid indicated at that position in sequence SEQ ID NO: 2, except K or R.

68. The pharmaceutical composition of any one of embodiments 54-67, wherein the VSV-G polypeptide further comprises a mutation that corresponds to position 10 in SEQ ID NO: 2, wherein the mutation is any amino acid different from the amino acid indicated at that position in sequence SEQ ID NO: 2, except Q or N.

69. The pharmaceutical composition of any one of embodiments 54-68, wherein the VSV-G polypeptide comprises a substitution at position 47 or at position 354, or at both positions 47 and 354, wherein each position is, independently, substituted by A, G, F, Q, or N.

70. The pharmaceutical composition of any one of embodiments 54-69, wherein the VSV-G polypeptide comprises a substitution at position 8, wherein the substitution is H8A, H8I, H8V, H8L, and the like.

71. The pharmaceutical composition of any one of embodiments 54-70, wherein the VSV-G polypeptide comprises a substitution at position 47, wherein the substitution is K47Q or K47N.

72. The pharmaceutical composition of any one of embodiments 54-71, wherein the VSV-G polypeptide comprises a substitution H8A and/or K47Q mutation.

73. The pharmaceutical composition of any one of embodiments 54-72, wherein the VSV-G polypeptide comprises a substitution at position 10, such as Q10A, Q10R, or Q10K substitution.

74. The pharmaceutical composition of any one of embodiments 54-73, wherein the VSV-G polypeptide, further comprises a mutation that corresponds to a mutation at positions 214 and/or 352 of SEQ ID NO: 2.

75. The pharmaceutical composition of embodiment 74, wherein the VSV-G polypeptide comprises a T214N and/or T352A mutation.

76. The pharmaceutical composition of any one of embodiments 74-75, wherein the VSV-G polypeptide comprises a substitution at positions I182 and at least one of T214, and T352 of SEQ ID NO: 2.

77. The pharmaceutical composition of any one of embodiments 74-76, wherein the VSV-G polypeptide comprises substitutions at positions I182, T214, and T352 of SEQ ID NO: 2.

78. The pharmaceutical composition of any one of embodiments 74-77, wherein the substitution at position 182 of the VSV-G polypeptide is I182D or I182E, the substitution at position 214 of the VSV-G polypeptide is T214N, and the substitution at position 352 of the VSV-G polypeptide is T352A.

79. The pharmaceutical composition of any one of embodiments 76-78, wherein the VSV-G polypeptide, comprises a sequence of SEQ ID NO: 22, SEQ ID NO: 23, SEQ ID NO: 24 or SEQ ID NO: 25.

80. The pharmaceutical composition of any one of embodiments 76-78, wherein the VSV-G polypeptide comprises a sequence of SEQ ID NO: 22.

81. The pharmaceutical composition of any one of embodiments 76-78, wherein the VSV-G polypeptide comprises a sequence of SEQ ID NO: 23.

82. The pharmaceutical composition of any one of embodiments 76-78, wherein the VSV-G polypeptide comprises a sequence of SEQ ID NO: 24.

83. The pharmaceutical composition of any one of embodiments 76-78, wherein the VSV-G polypeptide comprises a sequence of SEQ ID NO: 25.

84. A pharmaceutical composition comprising:
(i) a VSV-G pseudotyped lentivirus comprising a VSV-G polypeptide comprising a mutation that corresponds to a mutation at position 182, 214, and/or 352 of SEQ ID NO: 2;
(ii) L-Histidine buffer at a concentration of about 10 mM to about 30 mM;
(iii) sodium chloride at a concentration of about 100 mM to about 400 mM;
(iv) sucrose in amount of about 1% (w/v) to about 5% (w/v); and
(v) non-recombinant human serum albumin in an amount of about 0.1% (w/v) to about 2% (w/v), wherein the pharmaceutical composition has a pH of about 6.5 to about 7.5.

85. The pharmaceutical composition of any one of embodiments 1-84, wherein the lentiviral vector further comprises a targeting moiety.

86. The pharmaceutical composition of embodiment 85, wherein the targeting moiety binds to CD7.

87. The pharmaceutical composition of embodiment 86, wherein the targeting moiety comprises a polypeptide comprising: (i) a heavy chain variable region comprising heavy chain CDR1, CDR2, and CDR3 sequences, wherein the heavy chain CDR1 sequence has the amino acid sequence of SEQ ID NO: 30; the heavy chain CDR2 has the amino acid sequence of SEQ ID NO: 31; and the heavy chain CDR3 sequence has the amino acid sequence of SEQ ID NO: 32, or variants of any of the foregoing; and (ii) a light chain variable region comprising light chain CDR1, CDR2, and CDR3 sequences, wherein the light chain CDR1 sequence has the amino acid sequence of SEQ ID NO: 33; the light chain CDR2 sequence has the amino acid sequence of SEQ ID NO: 34; and the light chain CDR3 sequence has the amino acid sequence of SEQ ID NO: 35; or variants of any of the foregoing.

88. The pharmaceutical composition of embodiment 86 or 87, wherein the targeting moiety comprises a heavy chain variable region (VH) comprising an amino acid sequence of SEQ ID NO: 42 and a light chain variable region (VL) comprising an amino acid sequence of SEQ ID NO: 43.

89. The pharmaceutical composition of any one of embodiments 86-88, wherein the targeting moiety comprises an antigen binding domain comprising an amino acid sequence of SEQ ID NO: 44 or SEQ ID NO: 45.

90. The pharmaceutical composition of embodiment 85, wherein the targeting moiety binds to CD8.

91. The pharmaceutical composition of embodiment 90, wherein the targeting moiety comprises a polypeptide comprising: (i) a heavy chain variable region comprising heavy chain CDR1, CDR2, and CDR3 sequences, wherein the heavy chain CDR1 sequence has the amino acid sequence of SEQ ID NO: 48; the heavy chain CDR2 has the amino acid sequence of SEQ ID NO: 49; and the heavy chain CDR3 sequence has the amino acid sequence of SEQ ID NO: 50, or variants of any of the foregoing; and (ii) a light chain variable region comprising light chain CDR1, CDR2, and CDR3 sequences, wherein the light chain CDR1 sequence has the amino acid sequence of SEQ ID NO: 51; the light chain CDR2 sequence has the amino acid sequence of SEQ ID NO: 52; and the light chain CDR3 sequence has the amino acid sequence of SEQ ID NO: 53; or variants of any of the foregoing.

92. The pharmaceutical composition of embodiment 90 or 91, wherein the targeting moiety comprises a heavy chain variable region (VH) comprising an amino acid sequence of SEQ ID NO: 60 and a light chain variable region (VL) comprising an amino acid sequence of SEQ ID NO: 61.

93. The pharmaceutical composition of any one of embodiments 90-92, wherein the targeting moiety comprises an antigen binding domain comprising an amino acid sequence of SEQ ID NO: 62 or SEQ ID NO: 63.

94. The pharmaceutical composition of any one of embodiments 85-93, wherein the targeting moiety comprises a formula of T-S1, wherein T is a target binding domain and S₁ is a stalk portion, wherein the targeting moiety is attached to the surface of the viral vector through a stalk portion, S₁.

95. The pharmaceutical composition of embodiment 94, wherein the stalk portion, S₁ comprises a variant Fc protein.

96. The pharmaceutical composition of embodiment 95, wherein the stalk portion, S₁, comprises a formula of L₁-Fc-L₂-X₁, wherein:
L₁ is a linker or absent;
   Fc is a variant Fc protein;
   L₂ is a linker or absent; and
X₁ is a polypeptide comprising the transmembrane domain,
wherein the targeting moiety having the formula T-S₁ has a formula of T-L₁-Fc-L₂-X₁.

97. The pharmaceutical composition of embodiment 96, wherein X₁ comprises a polypeptide having a formula of ECD-T_{M}-ICD, wherein:
ECD is an extracellular domain, or a fragment thereof, of a cell surface protein, or absent;

T_{M} is a transmembrane domain of a transmembrane protein; and

ICD is an intracellular domain or a protein that facilitates incorporation of the targeting moiety into the envelope of the viral particle, or absent,
wherein the targeting moiety having the formula of T-L₁-Fc-L₂-X₁ has a formula of T-L₁-Fc-L₂-ECD-T_{M}-ICD.

98. The pharmaceutical composition of embodiment 97, wherein:
T comprises an amino acid sequence of SEQ ID NO: 44, SEQ ID NO: 45, SEQ ID NO: 62, or SEQ ID NO: 63;
L₁ comprises the amino acid sequence of SEQ ID NO: 72;
Fc comprises the amino acid sequence of SEQ ID NO: 82;
L₂ is absent;
ECD comprises the amino acid sequence of SEQ ID NO: 83;
T_{M} comprises the amino acid sequence of SEQ ID NO: 84; and
ICD comprises an amino acid sequence comprising an env incorporation motif,
wherein the env incorporation motif comprises an amino acid sequence of SEQ ID NO: 85.

99. The pharmaceutical composition of embodiment 94, wherein the stalk portion, S₁ does not comprise a variant Fc protein.

100. The pharmaceutical composition of embodiment 99, wherein the stalk portion S₁ comprises a formula of L₃-X₁, wherein:
L₃ is a flexible peptide linker, and
X₁ is a polypeptide comprising a transmembrane domain,
wherein the targeting moiety having the formula T-S₁ has a formula of T-L₃-X₁.

101. The pharmaceutical composition of embodiment 100, wherein X₁ comprises a polypeptide having a formula of ECD-T_{M}-ICD, wherein:
ECD is an extracellular domain, or a fragment thereof, of a cell surface protein, or absent;
T_{M} is a transmembrane domain of a transmembrane protein; and ICD is an intracellular domain or a protein that facilitates incorporation of the targeting moiety into the envelope of the viral particle, or absent,
wherein the targeting moiety having the formula of T-L₃-X₁ has a formula of T-L₃-ECD-T_{M}-ICD.

102. The pharmaceutical composition of embodiment 101, wherein:
T comprises an amino acid sequence of SEQ ID NO: 44, SEQ ID NO: 45, SEQ ID NO: 62, or SEQ ID NO: 63;
L₃ comprises the amino acid sequence of SEQ ID NO: 64, wherein n is 1, 2, or 4;
ECD comprises the amino acid sequence of SEQ ID NO: 86;
T_{M} comprises the amino acid sequence of SEQ ID NO: 87; and
ICD comprises an amino acid sequence comprising an env incorporation motif,
wherein the env incorporation motif comprises an amino acid sequence of SEQ ID NO: 85 or SEQ ID NO: 88.

103. The pharmaceutical composition of embodiment 94, wherein the targeting moiety comprising a formula of T-S₁ comprises an amino acid sequence having at least 90% identity to SEQ ID NO: 89.

104. The pharmaceutical composition of embodiment 94, wherein the targeting moiety comprising a formula of T-S₁ comprises an amino acid sequence having at least 95% identity to SEQ ID NO: 89.

105. The pharmaceutical composition of embodiment 94, wherein the targeting moiety comprising a formula of T-S₁ comprises an amino acid sequence of SEQ ID NO: 89.

106. The pharmaceutical composition of any one of embodiments 1-105, wherein the lentiviral vector further comprises a nucleic acid molecule encoding for a heterologous molecule of interest.

107. The pharmaceutical composition of embodiment 106, wherein the heterologous molecule of interest is an siRNA, an shRNA, a non-coding RNA (e.g. a guide RNA for a CRISPR system), a peptide, a polypeptide, a protein, a viral payload, a viral genome, or a combination thereof.

108. The pharmaceutical composition of embodiment 106 or 107, wherein the heterologous molecule of interest is a chimeric antigen receptor (“CAR”).

109. The pharmaceutical composition of embodiment 108, wherein the CAR comprises an antigen binding domain comprising an antibody, or a fragment thereof.

110. The pharmaceutical composition of embodiment 109, wherein the antibody, or fragment thereof is an antibody, a scFv antibody, an antigen binding domain, an ankyrin repeat, a VHH domain antibody, a nanobody, a single domain antibody, or an FN3 antibody.

111. The pharmaceutical composition of embodiment 109 or 110, wherein the antigen binding domain of the CAR binds to CD20.

112. The pharmaceutical composition of embodiment 111, wherein the antigen binding domain that binds to CD20 comprises a polypeptide comprising a light chain and a heavy chain comprising: a heavy chain variable region of the heavy chain having at least 90% identity to an amino acid sequence of SEQ ID NO: 73; and a light chain variable region of the light chain having at least 90% identity to an amino acid sequence of SEQ ID NO: 74.

113. The pharmaceutical composition of embodiment 111 or 112, wherein the antigen binding domain that binds to CD20 comprises an amino acid sequence of SEQ ID NO: 75 or SEQ ID NO: 76.

114. The pharmaceutical composition of embodiment 111, wherein the antigen binding domain that binds to CD20 comprises a polypeptide comprising a light chain and a heavy chain comprising: a heavy chain variable region of the heavy chain having at least 90% identity to an amino acid sequence of SEQ ID NO: 78; and a light chain variable region of the light chain having at least 90% identity to an amino acid sequence of SEQ ID NO: 79.

115. The pharmaceutical composition of embodiment 114, wherein the antigen binding domain that binds to CD20 comprises an amino acid sequence of SEQ ID NO: 80 or SEQ ID NO: 81.

116. The pharmaceutical composition of any one of embodiments 108-111, wherein the CAR comprises an amino acid sequence having at least 90% identity to SEQ ID NO: 90.

117. The pharmaceutical composition of any one of embodiments 108-111, wherein the CAR comprises an amino acid sequence having at least 95% identity to SEQ ID NO: 90.

118. The pharmaceutical composition of any one of embodiments 108-111, wherein the CAR comprises an amino acid sequence of SEQ ID NO: 90.

119. A method of infecting a cell, the method comprising contacting the cell with the pharmaceutical composition of any one of embodiments 1-118.

120. A method of infecting a cell in a subject, the method comprising administering to the subject the pharmaceutical composition of any one of embodiments 1-118.

121. A method of delivering a heterologous molecule of interest to a cell, the method comprising contacting the cell with the pharmaceutical composition of any one of embodiments 1-118, wherein the lentiviral vector of the pharmaceutical composition comprises a nucleic acid molecule encoding the heterologous molecule of interest.

122. A method of delivering a heterologous molecule of interest to a subject, the method comprising administering to the subject the viral particle of any one of embodiments 1-118, wherein the lentiviral vector of the pharmaceutical composition comprises a nucleic acid molecule encoding the heterologous molecule of interest.

123. The method of any one of embodiments 119-122, wherein the cell is an immune cell.

124. The method of any one of embodiments 119-123, wherein the cell is a T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD20+ cancer cell, CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte.

125. A method of treating a disease or disorder in a subject, the method comprising administering to the subject the pharmaceutical composition of any one of embodiments 1-118, wherein the lentiviral vector of the pharmaceutical composition comprises a nucleic acid molecule encoding a heterologous molecule of interest to treat the disease or disorder.

126. The method of embodiment 125, wherein the disease or disorder is cancer (e.g. such as a T cell or B cell disorder or other type of cancer as described herein), an immune disorder, an auto-immune disorder, metabolic disorder, and the like.

127. A method of delivering a heterologous molecule to a target cell, the method comprising contacting the cell with the pharmaceutical composition of any one of embodiments 1-118, wherein the lentiviral vector of the pharmaceutical composition comprises a nucleic acid molecule encoding the heterologous molecule.

128. The pharmaceutical composition of any one of embodiments 1-118, for use in the treatment of a cancer, an immune disorder, an auto-immune disorder, or a metabolic disorder.

129. Use of the pharmaceutical composition of any one of claims 1-118, for the manufacture of a medicament for treatment of a cancer, an immune disorder, an auto-immune disorder, or a metabolic disorder.

### Examples

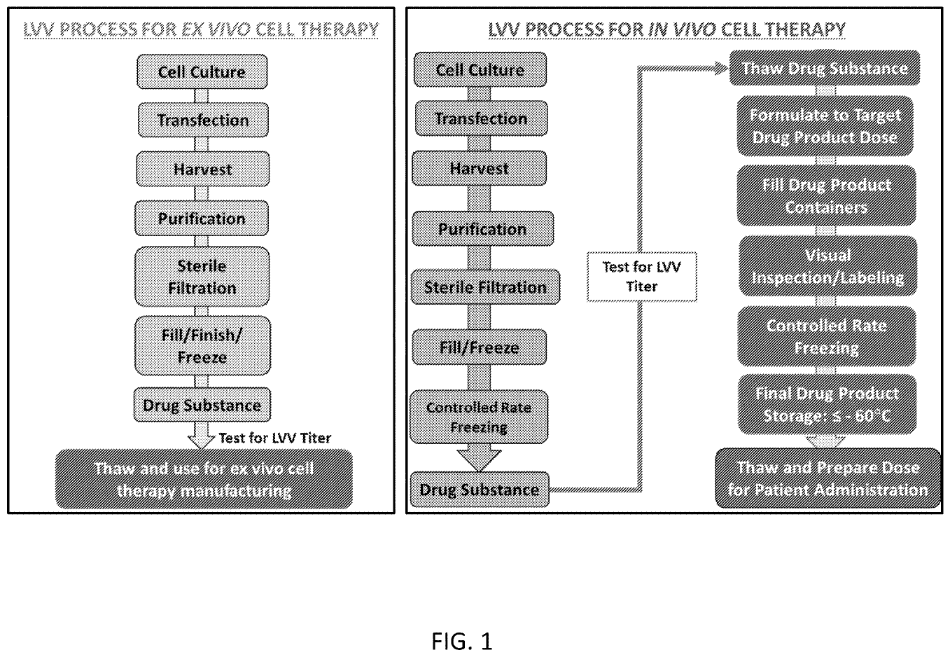

Example 1: LV Process for ex vivo and in vivo use. For ex vivo cell therapy, the lentiviral vector processing involves a single freeze/thaw step after sterile filtration to obtain the drug substance for cell therapy manufacturing. For in vivo cell therapy, however, two separate instances of controlled rate freezing are necessary to obtain the optimal final drug product that is suitable for patient administration (FIG. 1).

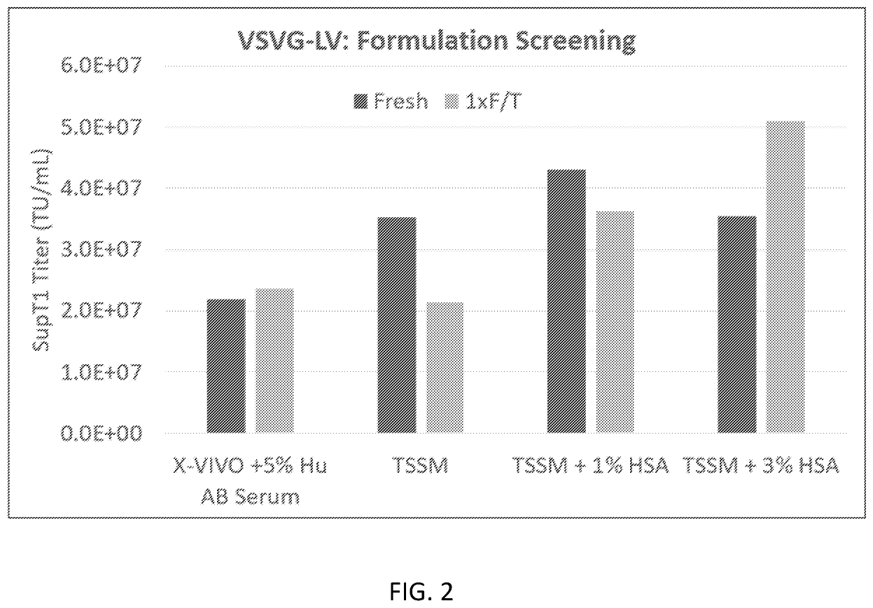

Example 2: Formulation Screening. To improve upon previously reported literature utilizing TSSM (20 mM Tris, 100 mM NaCl, 1% Sucrose, 1% Mannitol) formulation buffer for VSVG-pseudotyped lentiviral vectors, a formulation screening was conducted. By testing TSSM buffer with or without a range of HSA concentrations it was determined that a protein excipient (human serum or human serum albumin) was ideal for freeze/thaw stability (FIG. 2.).

Example 3: Formulation Development: multiple freeze-thaw cycles. In order to develop a formulation that is compatible for intravenous and intralymphatic administration, the lentivirus must be stabilized through two freeze-thaw cycles. A subset of formulations were tested with both one and two freeze-thaw cycles to assess potential loss of transduction efficiency post freeze-thaw cycle. Formulations tested included TSSM (20 mM Tris, 100 mM NaCl, 1% Sucrose, 1% Mannitol), TSS (20 mM Tris, 100 mM NaCl, 1% Sucrose), and HSS (20 mM Histidine, 100 mM NaCl, 4% Sucrose) at a range of pH levels and protein excipient concentrations. For each formulation and condition tested, the input concentration of lentivirus was identical. Formulations tested are provided in Table 5 below:

TABLE 5

| | | | | Protein | |
|---|---|---|---|---|---|
| Formulation | Buffer | Salt | Sugar | excipient | pH |
| 1 | 20 mM Tris | 100 mM | 1% Sucrose, | | 7.3 |
| | | NaCl | 1% Mannitol | | |
| 2 | 20 mM Tris | 100 mM | 1% Sucrose, | 3% HSA | 7.3 |
| | | NaCl | 1% Mannitol | | |
| 3 | 20 mM Tris | 100 mM | 1% Sucrose, | 3% HSA | 7.3 |
| | | NaCl | | | |
| 4 | 20 mM Tris | 100 mM | 1% Sucrose, | 1% | 7.3 |
| | | NaCl | 1% Mannitol | Exbumin ® | |
| 5 | 20 mM Tris | 100 mM | 1% Sucrose, | 3% | 7.3 |
| | | NaCl | 1% Mannitol | Exbumin ® | |
| 6 | 20 mM Tris | 100 mM | 1% Sucrose | 3% | 7.3 |
| | | NaCl | | Exbumin ® | |
| 7 | 20 mM Tris | 100 mM | 1% Sucrose | 3% HSA | 7.3 |
| | | NaCl | | | |
| 8 | 20 mM Tris | 100 mM | 1% Sucrose | | 7.3 |
| | | NaCl | | | |
| 9 | 20 mM Tris | 100 mM | 1% Sucrose | | 8.0 |
| | | NaCl | | | |
| 10 | 20 mM Tris | 100 mM | 1% Sucrose | | 8.5 |
| | | NaCl | | | |
| 11 | 20 mM Tris | 100 mM | 2% Sucrose | | 7.3 |
| | | NaCl | | | |
| 12 | 20 mM Tris | 100 mM | 4% Sucrose | | 7.3 |
| | | NaCl | | | |
| 13 | 20 mM Tris | 100 mM | 4% Sucrose | 3% HSA | 7.3 |
| | | NaCl | | | |
| 14 | 20 mM Tris | 100 mM | 1% Sucrose, | | 7.3 |
| | | NaCl | 1% Mannitol | | |
| 15 | 20 mM Tris | 100 mM | 4% Sucrose | | 7.3 |
| | | NaCl | | | |
| 16 | 20 mM Tris | 100 mM | 4% Sucrose | 3% | 7.3 |
| | | NaCl | | Exbumin ® | |
| 17 | 20 mM Tris | 100 mM | 4% Sucrose, | | 8.0 |
| | | NaCl | | | |
| 18 | 20 mM | 100 mM | 4% Sucrose | | 6.0 |
| | Histidine | NaCl | | | |
| 19 | 20 mM | 100 mM | 4% Sucrose | | 6.5 |
| | Histidine | NaCl | | | |
| 20 | 20 mM | 100 mM | 4% Sucrose | | 7.0 |
| | Histidine | NaCl | | | |

The results are shown in FIG. 3. In FIG. 3 and Table 5 above, duplicate formulations represent unique vector preparations. The data illustrate the minimal to no loss observed across the additional freeze-thaw cycle (comparing 1×F/T data to 2×F/T data). In assessing the differences in formulation components, the data indicated that Mannitol was not necessary for virus stability. Further, the data indicate that both recombinant human albumin (Exbumin®) and non-recombinant human serum albumin behave similarly with respect to maintaining transduction efficiency across additional freeze-thaw cycles.

Example 4: Formulation Confirmation Study. Lead formulations were tested under different temperatures and handling conditions. The formulations tested were as provided in Table 6 below:

TABLE 6

| | | | | Protein | |
|---|---|---|---|---|---|
| Formulation | Buffer | Salt | Sugar | excipient | pH |
| 21 | 20 mM Tris | 100 mM | 4% Sucrose | | 7.3 |
| | | NaCl | | | |
| 22 | 20 mM Tris | 100 mM | 2% Sucrose, | | 7.3 |
| | | NaCl | 1% Mannitol | | |
| 23 | 20 mM Tris | 100 mM | 2% Sucrose, | 3% HSA | 7.3 |
| | | NaCl | 1% Mannitol | | |
| 24 | 20 mM Tris | 100 mM | 4% Sucrose | 1% | 7.3 |
| | | NaCl | | Exbumin ® | |
| 25 | 20 mM | 100 mM | 4% Sucrose | | 7.0 |
| | Histidine | NaCl | | | |

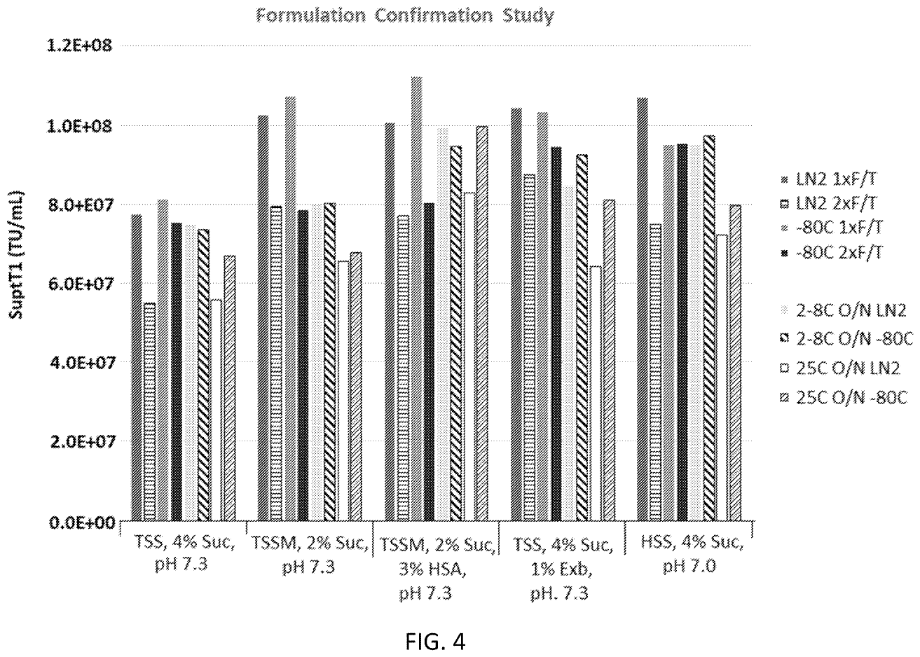

As shown in FIG. 4, the formulations tested showed a similar stability profile. Histidine buffer was selected for better pH control at pH 7.0 for further optimization. The data also show that the addition of the protein excipient provides better overall stability (FIG. 4).

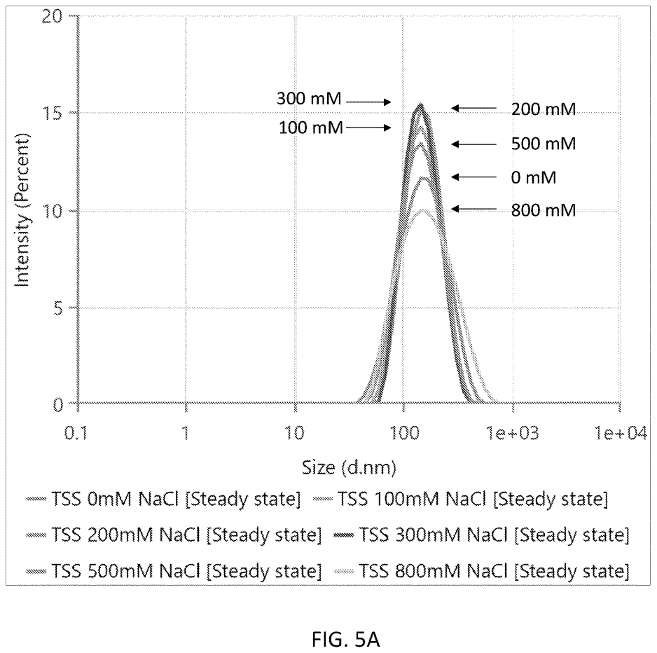

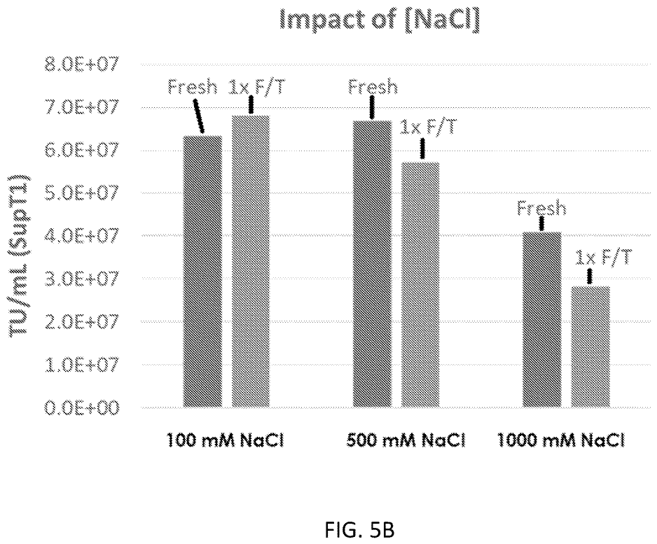

Example 5: Optimal [NaCl] can help minimize LV aggregation. The polydispersity index (PDI) was measured by DLS for TSS buffer under increasing concentrations of NaCl. The data provide an indication of heterogeneity of the particles. Optimal [NaCl]/ionic strength can help minimize particle aggregation (by charge shielding) without impacting LV stability, as illustrated by comparing the impact of increasing concentrations of NaCl between fresh sample and sample undergoing a single freeze/thaw cycle (FIG. 5). The formulations tested in FIG. 5 are as provided in Table 7 below:

TABLE 7

| | | | | Protein | |
|---|---|---|---|---|---|
| Formulation | Buffer | Salt | Sugar | excipient | pH |
| 26 | 20 mM Tris | | 1 % Sucrose | | |
| 27 | 20 mM Tris | 100 mM | 1 % Sucrose | | |
| | | NaCl | | | |
| 28 | 20 mM Tris | 200 mM | 1 % Sucrose | | |
| | | NaCl | | | |
| 29 | 20 mM Tris | 300 mM | 1% Sucrose | | |
| | | NaCl | | | |
| 30 | 20 mM Tris | 500 mM | 1% Sucrose | | |
| | | NaCl | | | |
| 31 | 20 mM Tris | 800 mM | 1 % Sucrose | | |
| | | NaCl | | | |
| 32 | 20 mM Tris | 1 M NaCl | 1 % Sucrose | | |

Example 6: HSS Formulation Optimization. HSS formulation optimization was done to identify optimal [NaCl]/ionic strength to help minimize particle aggregation (by charge shielding) without impacting LV stability, as well as evaluating the impact of including recombinant human albumin. HSS at a range of concentrations at a pH of 6.5 or 7.0 with or without 1% Exbumin® were tested at various temperatures and handling conditions. The formulations tested are as provided in Table 8 below:

TABLE 8

| | | | | Protein | |
|---|---|---|---|---|---|
| Formulation | Buffer | Salt | Sugar | excipient | pH |
| 33 | 20 mM | 100 mM | 4% Sucrose | | 7.0 |
| | Histidine | NaCl | | | |
| 34 | 20 mM | 150 mM | 4% Sucrose | | 7.0 |
| | Histidine | NaCl | | | |
| 35 | 20 mM | 200 mM | 4% Sucrose | | 7.0 |
| | Histidine | NaCl | | | |
| 36 | 20 mM | 100 mM | 4% Sucrose | 1% | 7.0 |
| | Histidine | NaCl | | Exbumin ® | |
| 37 | 20 mM | 150 mM | 4% Sucrose | 1% | 7.0 |
| | Histidine | NaCl | | Exbumin ® | |
| 38 | 20 mM | 200 mM | 4% Sucrose | 1% | 7.0 |
| | Histidine | NaCl | | Exbumin ® | |
| 39 | 20 mM | 150 mM | 4% Sucrose | 1% | 6.5 |
| | Histidine | NaCl | | Exbumin ® | |

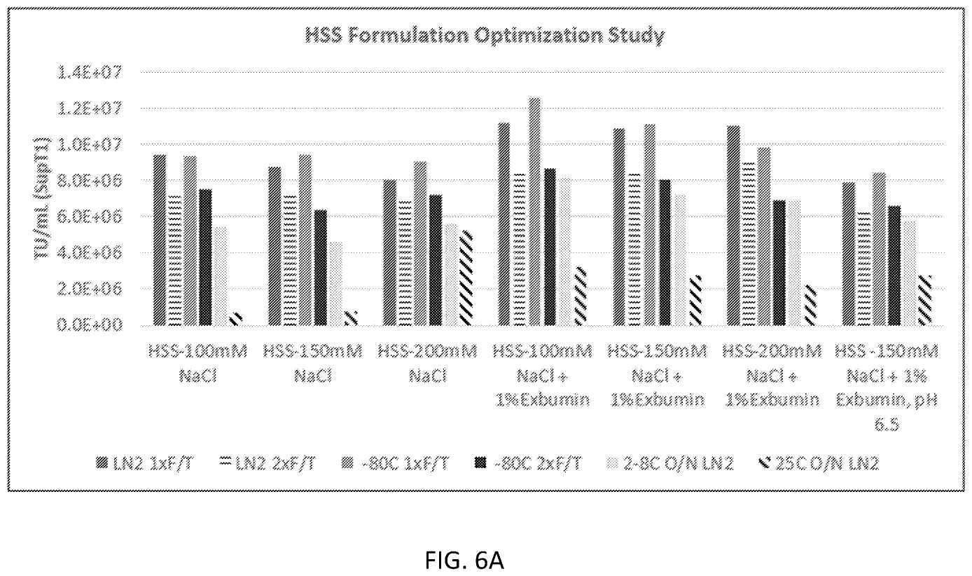

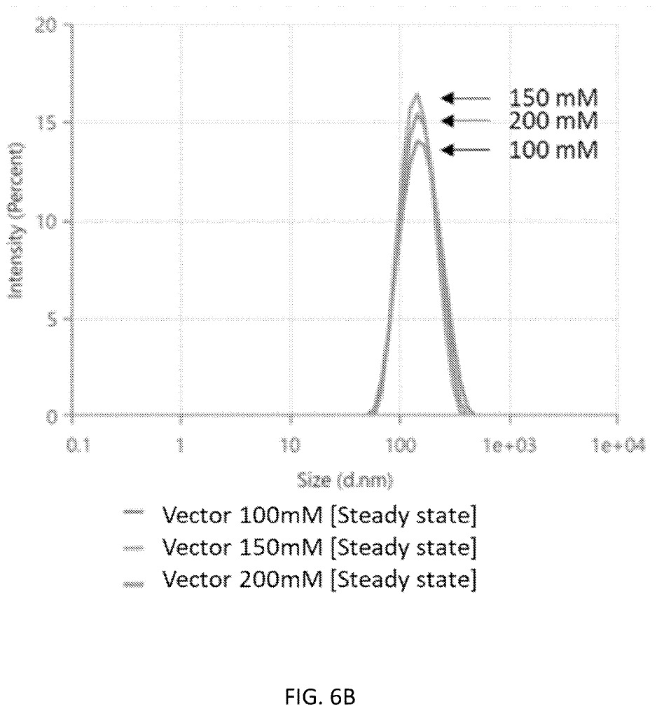

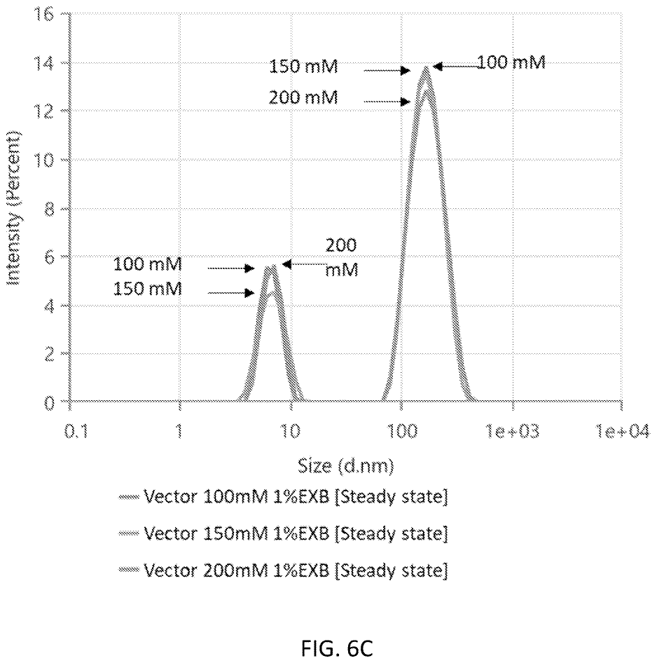

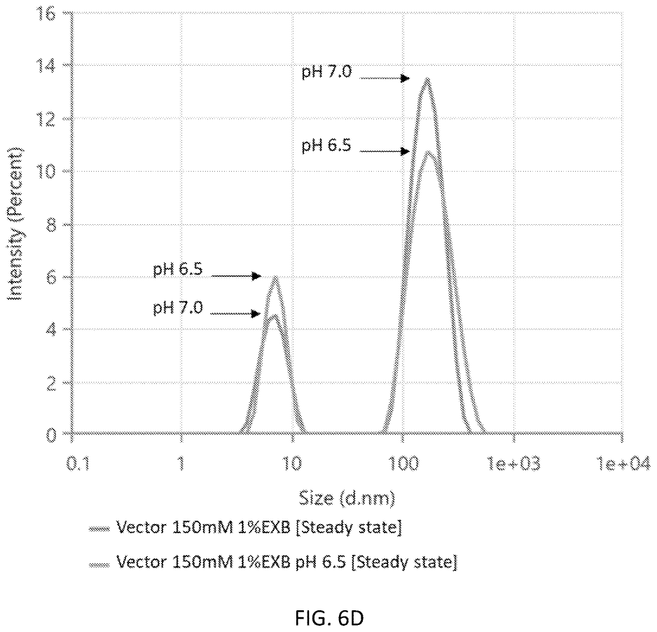

The data show that including 1% Exbumin® shows better overall stability, and higher [NaCl] does not impact functional titer, but does show more uniform particle size distribution (FIG. 6A-6D).

Example 7: Evaluation of Formulation Stability: Recombinant vs. non-recombinant HSA. The effect of the source of human serum albumin was assessed by comparing the stability of formulations containing either 0.5% recombinant human serum albumin (Exbumin®) or 0.5% non-recombinant, USP grade human serum albumin. The formulations tested are as provided in Table 9 below:

TABLE 9

| | | | | Protein | |
|---|---|---|---|---|---|
| Formulation | Buffer | Salt | Sugar | excipient | pH |
| 40 | 20 mM | 150 mM | 4% Sucrose | 0.5% | 7.0 |
| | Histidine | NaCl | | Exbumin ® | |
| 41 | 20 mM | 150 mM | 4% Sucrose | 0.5% HSA | 7.0 |
| | Histidine | NaCl | | | |

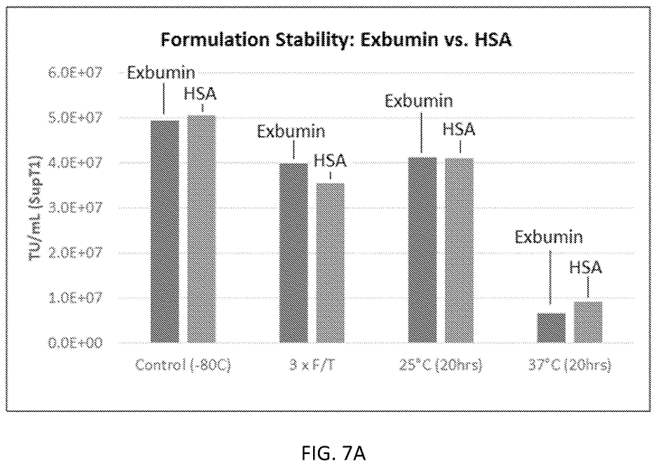

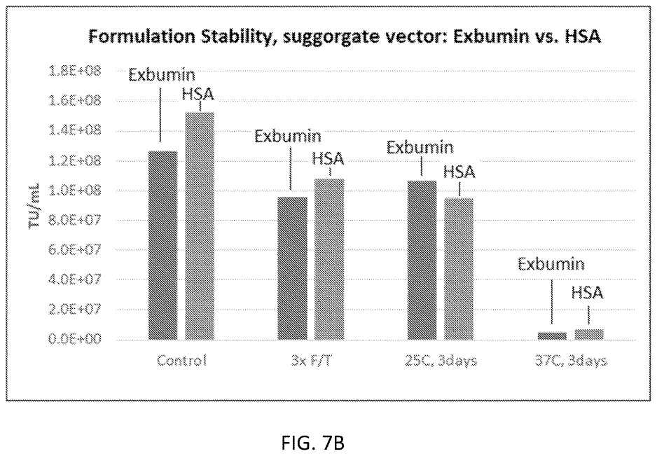

The two preparations were subject to various stress conditions and the stability was measured via viral titer (FIG. 7A). The two preparations had similar stability profiles when subject to three freeze thaw cycles, room temperature storage for 20 hours, or storage at 37° C. for 20 hours. The experiment was repeated, but the viral vector was substituted with a non-human primate surrogate vector (FIG. 7B). Again, the two preparations had similar stability profiles when subject to three freeze thaw cycles, room temperature storage for three days, and storage at 37° C. for three days. These results indicate that replacing the recombinant human serum albumin (Exbumin®) with non-recombinant USP grade HSA had no significant impact on the stability of the viral vector preparations.

Example 8: Mutation at position 182 of VSV-G abrogates LDL-R interaction, but retains fusing properties. Plasmids/Sequences. All VSV-G plasmids were derived from pCMV-VSV-G Envelope Vector (Cell Bio Labs, catalog RV-110). Point mutations and combinations thereof were introduced using site directed mutagenesis (New England Biolabs). Individual mutations H8A and K47Q were previously shown to partially “blind” VSV-G, reducing its binding to LDL-R, the native cellular receptor for VSV (PMID: 29531262, DOI: 10.1038/s41467-018-03432-4). In this experiment, a single binder molecule consisting of a CD7-targeting scFv (clone MT701) fused to an IgG “stalk” bearing a CD28 transmembrane domain was used.

Cells. HEK293T cells were grown in DMEM with 10% FBS. SupT1 cells were maintained in RPMI media with 10% FBS. Human PBMCs were purchased from AllCells and cultured in X-Vivo 10 (Lonza) supplemented with 20 ng/ml IL-2 (Peprotech). PBMCs were activated 48 hours prior to transduction using anti-CD3/CD28 Dynabeads (Cell Therapy Systems).

Generation of lentiviral particles. The recombinant lentiviral particles co-expressing VSV-G glycoprotein and binder molecules were generated by plasmid transection into HEK293T cells using Lipofectamine 3000 (ThermoFisher Scientific). A total of 5 plasmids were transfected: (1) plasmid expressing the VSV-G glycoprotein, (2) plasmid expressing the binder protein (3) plasmid expressing the lentiviral transfer genome encoding for eGFP, (4) plasmid expressing gag-pol, and (5) plasmid expressing rev. Transfected cell supernatant was harvested 48 hours later. Virus in the cell supernatant was concentrated by centrifugation through a sucrose cushion and resuspended in PBS. Lentiviral particle titer was determined using the Lenti-X p24 Rapid Titer Kit (Takara Bio, San Jose, CA).

Lentivirus transduction assay. A series of 10-fold dilutions (in cell culture media) of the concentrated lentivirus was performed and used to infect SupT1 and activated human PBMCs. Media was replaced 6 hours later, and the transduced cells were analyzed by flow cytometry on days 4 and 7 after transduction. Cells were stained with a viability stain and an anti-CD7 antibody to detect CD7 positive cells (PeCy7 mouse-anti-human CD7, clone CD7-6B7, BD Biosciences). Expression of eGFP was measured to calculate transduction efficiency.

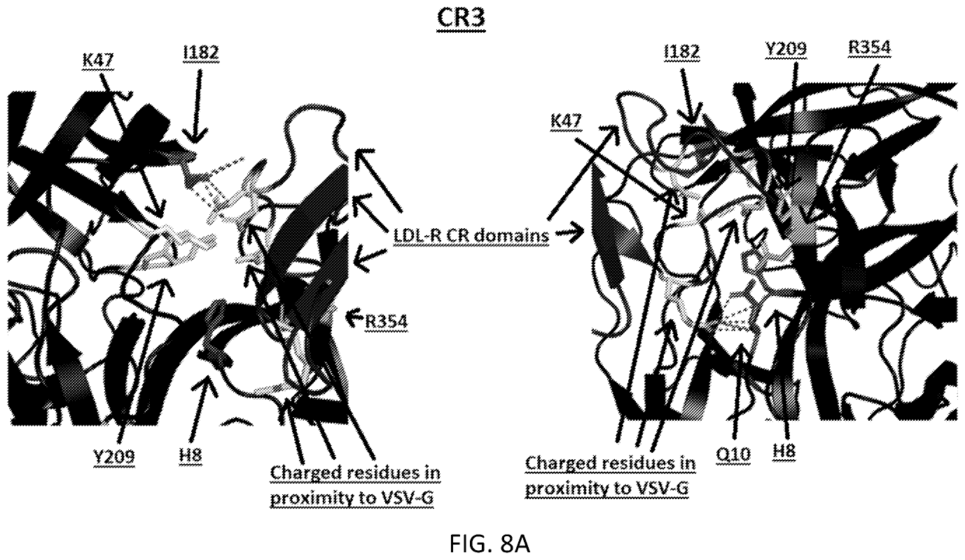

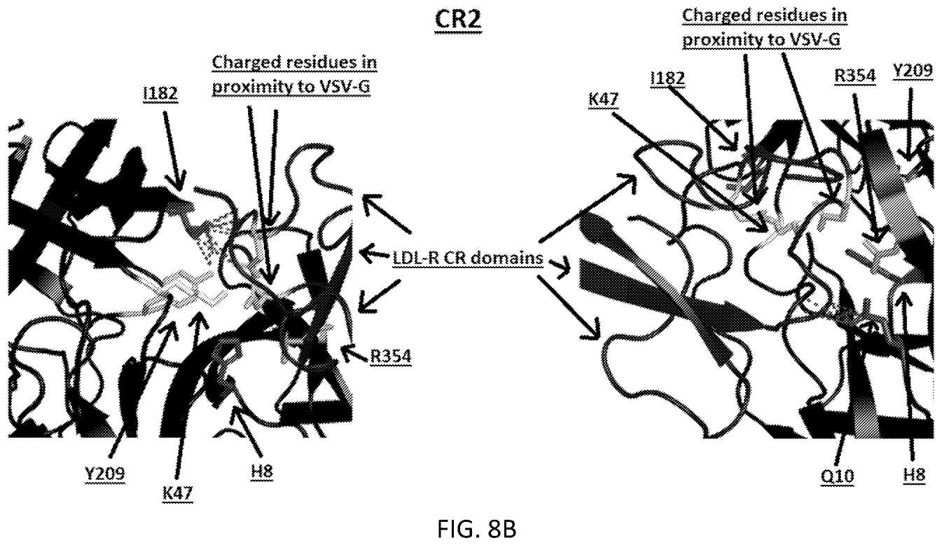

Structure-guided design of novel blinding mutations. Using a published crystal structures of VSV-G bound to CR2 and CR3 of the LDL-R (pdb 50YL and 50Y9, respectively), we identified two putative positions in VSV-G with side chains oriented toward the binding interface on LDL-R (FIG. 8A-B). Residue Q10 (SEQ ID NO: 2) appeared to form several interactions with residues in both CR2 and CR3. In CR3, this included interactions with a positively charged arginine residue. Thus, three substitutions were tested: Q10A to reduce side-chain interactions that potentially stabilize LDL-R binding and Q10R and Q10K to create electrostatic repulsion.

Residue I182 (SEQ ID NO: 2) appeared to contact several residues in both CR2 and CR3 as well. Three substitutions were tested: I182A to reduce side-chain interactions that potentially stabilize LDL-R binding and I182D and I182E to create electrostatic repulsion against the primary binding interfaces on LDL-R.

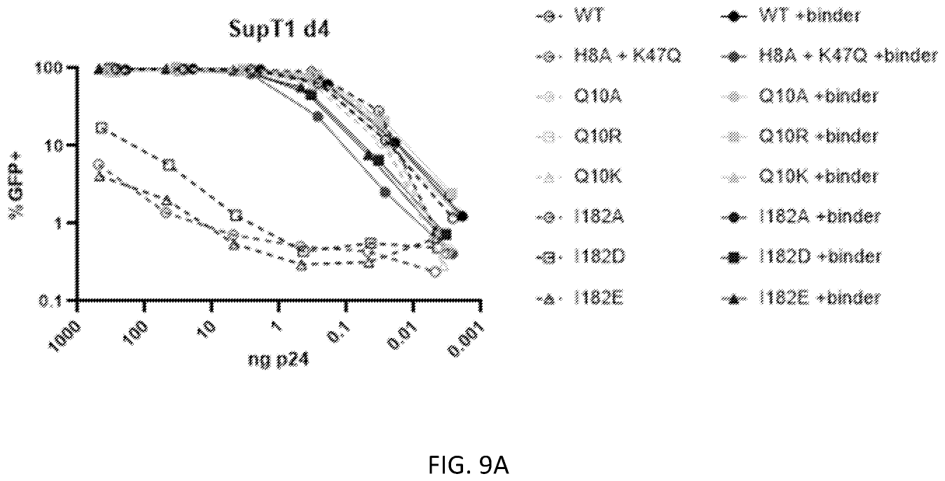

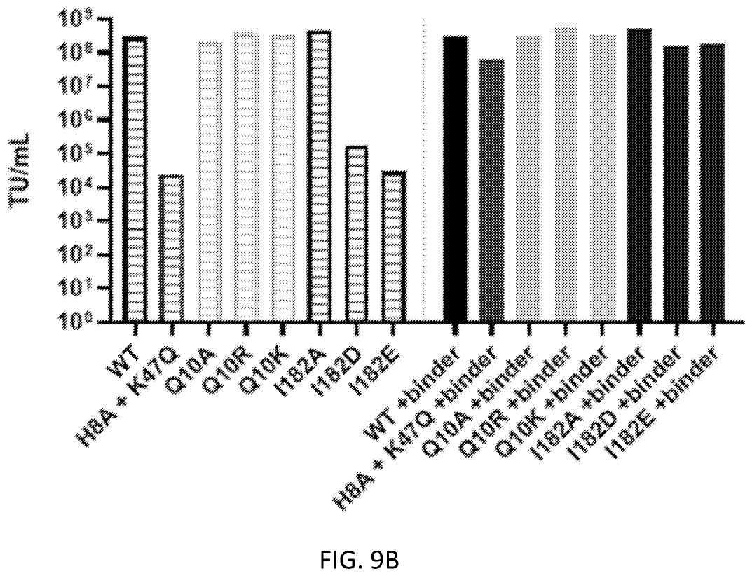

Addition of negative charges in the binding interface ablate native tropism without altering fusogenicity. Titration of viral supernatants on the CD7+ T cell line SupT1 validated the structural predictions for residue I182. In the absence of any compensatory binder molecule, WT VSV-G reached titers of 3.0e8 while both I182D and I182E were ~3 orders of magnitude lower (FIG. 9A). Substitutions at residue I182 preserved fusogenicity, as titers were restored to 1e8 in the presence of a binder redirecting the virions to CD7.

The data is illustrated in FIG. 9A, which shows that the addition of negative charges in the binding interface ablate native tropism without altering fusogenicity. FIG. 8a shows the titration of VSV-G constructs on SupT1 cells. Plotted is the percentage of SupT1 cells expressing GFP at each amount of viral input in terms of p24 antigen. Dashed lines/open circles indicate VSV-G constructs alone, solid lines/filled circles indicate the same construct with a CD7 targeting molecule expressed in trans. FIG. 9B illustrates Functional titer of each construct calculated from the titration in A, expressed as transducing units per mL of concentrated virus supernatant (TU/mL).

Thus these examples demonstrate that a mutation position 182 is sufficient to abrogate the LDL-R interaction, but retain fusogenic properties when combining with a targeting moiety that binds to a target on the target cell.

This specification contains numerous citations to patents, patent applications, and publications. Each is hereby incorporated by reference for all purposes.

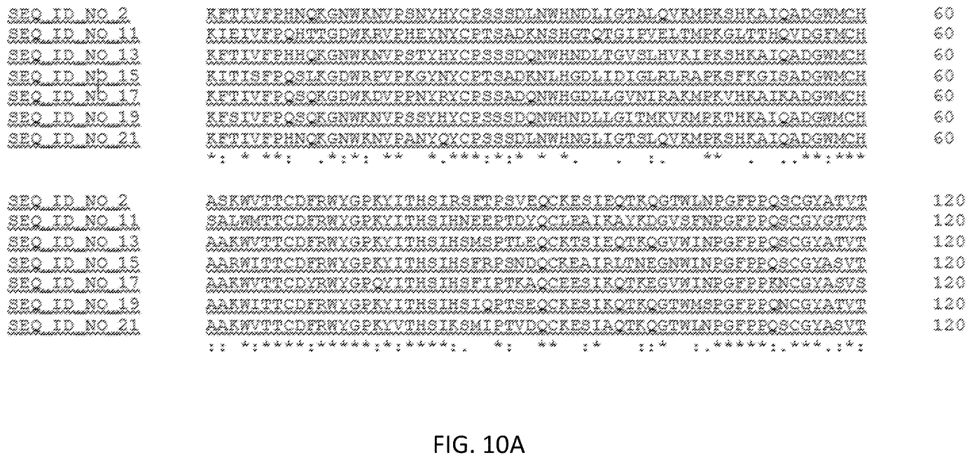

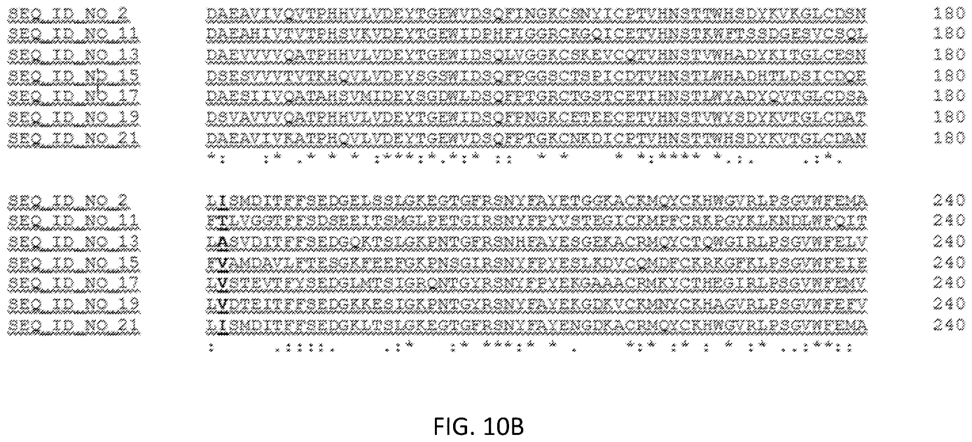

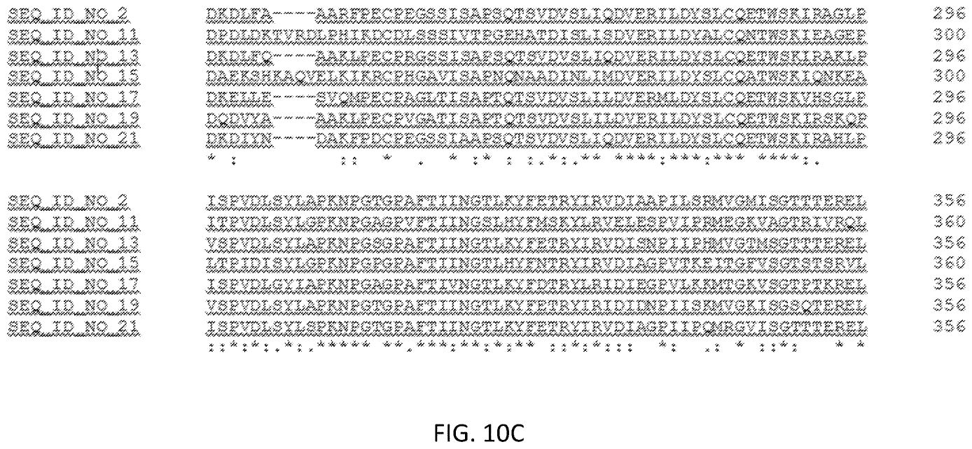

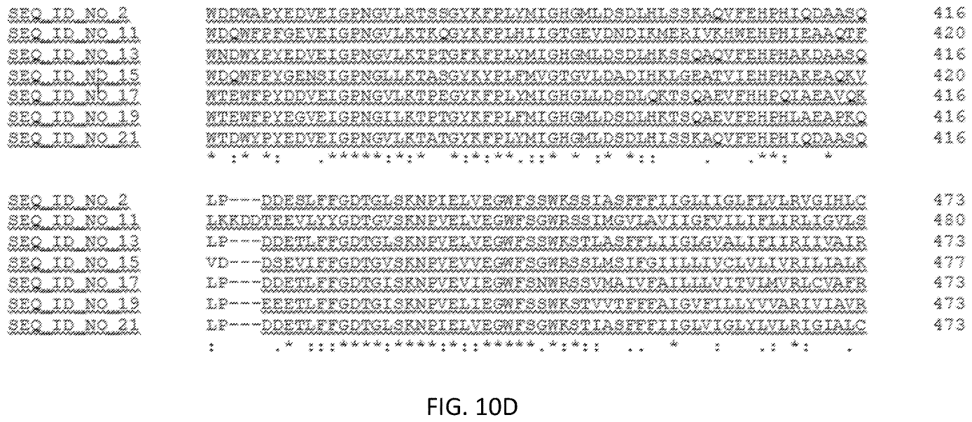

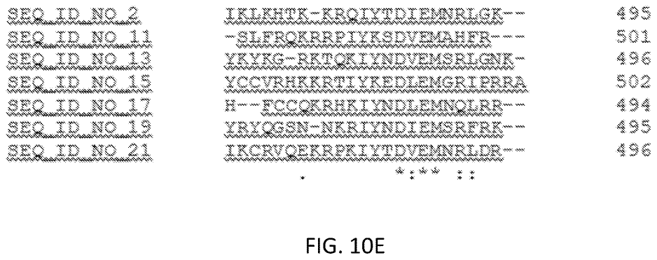

The specification also makes reference to various sequences, such as those provided herein and below. The alignment of the ectodomains of different VSV-G proteins from different strains is illustrated in FIGS. 10A, 10B, 10C, 10D, and 10E.

VSV-G Indiana Full length WT:
(SEQ ID NO: 1)
MKCLLYLAFLFIGVNCKFTIVFPHNQKGNWKNVPSNYHYCPSSSDLNWHNDLIGTALQVKMPKSHKAIQADGWMC
HASKWVTTCDFRWYGPKYITHSIRSFTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLISMDITFFSEDGELSSLGKEGTGFRSNY
FAYETGGKACKMQYCKHWGVRLPSGVWFEMADKDLFAAARFPECPEGSSISAPSQTSVDVSLIQDVERILDYSLC
QETWSKIRAGLPISPVDLSYLAPKNPGTGPAFTIINGTLKYFETRYIRVDIAAPILSRMVGMISGTTTERELWDD
WAPYEDVEIGPNGVLRTSSGYKFPLYMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQIYTDIEMNRLGK
VSV-G Indiana Ectodomain WT:
(SEQ ID NO: 2)
KFTIVFPHNQKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain I182A:
(SEQ ID NO: 3)
KFTIVFPHNQKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVTDAEAVIVQVTPHHVLVDEYTGEWVDSQFIN
GKCSNYICPTVHNSTTWHSDYKVKGLCDSNLASMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain I182D:
(SEQ ID NO: 4)
KFTIVFPHNQKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVTDAEAVIVQVTPHHVLVDEYTGEWVDSQFIN
GKCSNYICPTVHNSTTWHSDYKVKGLCDSNLDSMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain I182E:
(SEQ ID NO: 5)
KFTIVFPHNQKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVIDAEAVIVQVTPHHVLVDEYTGEWVDSQFIN
GKCSNYICPTVHNSTTWHSDYKVKGLCDSNLESMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain H8A + K47Q:
(SEQ ID NO: 6)
KFTIVFPANQKGNWKNVPSNYHYCPSSSDLNWHNDLIGTALQVKMPQSHKAIQADGWMCHASKWVTTCDFRWYGP
KYITHSIRSFTPSVEQCKESIEQTKQGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain Q10A:
(SEQ ID NO: 7)
KFTIVFPHNAKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain Q10R:
(SEQ ID NO: 8)
KFTIVFPHNRKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana ectodomain Q10K:
(SEQ ID NO: 9)
KFTIVFPHNKKGNWKNVPSNYHYCPSSSDLNWHNDLIGTALQVKMPKSHKAIQADGWMCHASKWVTTCDFRWYGP
KYITHSIRSFTPSVEQCKESIEQTKQGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGKEGTGFRSNYFAYETGGKACKMQYCK
HWGVRLPSGVWFEMADKDLFAAARFPECPEGSSISAPSQTSVDVSLIQDVERILDYSLCQETWSKIRAGLPISPV
DLSYLAPKNPGTGPAFTIINGTLKYFETRYIRVDIAAPILSRMVGMISGTTTERELWDDWAPYEDVEIGPNGVLR
TSSGYKFPLYMIGHGMLDSDLHLSSKAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G New Jersey Full length WT:
(SEQ ID NO: 10)
MLSYLIFALVVSPILGKIEIVFPQHTTGDWKRVPHEYNYCPTSADKNSHGTQTGIPVELTMPKGLTTHQVDGFMC
HSALWMTTCDFRWYGPKYITHSIHNEEPTDYQCLEAIKAYKDGVSFNPGFPPQSCGYGTVTDAEAHIVTVTPHSV
KVDEYTGEWIDPHFIGGRCKGQICETVHNSTKWFTSSDGESVCSQLFTLVGGTFFSDSEEITSMGLPETGIRSNY
FPYVSTEGICKMPFCRKPGYKLKNDLWFQITDPDLDKTVRDLPHIKDCDLSSSIVTPGEHATDISLISDVERILD
YALCQNTWSKIEAGEPITPVDLSYLGPKNPGAGPVFTIINGSLHYFMSKYLRVELESPVIPRMEGKVAGTRIVRQ
LWDQWFPFGEVEIGPNGVLKTKQGYKFPLHIIGTGEVDNDIKMERIVKHWEHPHIEAAQTFLKKDDTEEVLYYGD
TGVSKNPVELVEGWFSGWRSSIMGVLAVIIGFVILIFLIRLIGVLSSLFRQKRRPIYKSDVEMAHFR
VSV-G New Jersey ectodomain WT:
(SEQ ID NO: 11)
KIEIVFPQHTTGDWKRVPHEYNYCPTSADKNSHGTQTGIPVELTMPKGLTTHQVDGFMCHSALWMTTCDFRWYGP
KYITHSIHNEEPTDYQCLEAIKAYKDGVSFNPGFPPQSCGYGTVTDAEAHIVTVTPHSVKVDEYTGEWIDPHFIG
GRCKGQICETVHNSTKWFTSSDGESVCSQLFTLVGGTFFSDSEEITSMGLPETGIRSNYFPYVSTEGICKMPFCR
KPGYKLKNDLWFQITDPDLDKTVRDLPHIKDCDLSSSIVTPGEHATDISLISDVERILDYALCQNTWSKIEAGEP
ITPVDLSYLGPKNPGAGPVFTIINGSLHYFMSKYLRVELESPVIPRMEGKVAGTRIVRQLWDQWFPFGEVEIGPN
GVLKTKQGYKFPLHIIGTGEVDNDIKMERIVKHWEHPHIEAAQTFLKKDDTEEVLYYGDTGVSKNPVELVEGWFS
GWRSSIMGVLAVIIGFVILIFLIRLIGVLSSLFRQKRRPIYKSDVEMAHFR
VSV-G Marraba Full length WT:
(SEQ ID NO: 12)
MLRLFLFCFLALGAHSKFTIVFPHHQKGNWKNVPSTYHYCPSSSDQNWHNDLTGVSLHVKIPKSHKAIQADGWMC
HAAKWVTTCDFRWYGPKYITHSIHSMSPTLEQCKTSIEQTKQGVWINPGFPPQSCGYATVTDAEVVVVQATPHHV
LVDEYTGEWIDSQLVGGKCSKEVCQTVHNSTVWHADYKITGLCESNLASVDITFFSEDGQKTSLGKPNTGFRSNH
FAYESGEKACRMQYCTQWGIRLPSGVWFELVDKDLFQAAKLPECPRGSSISAPSQTSVDVSLIQDVERILDYSLC
QETWSKIRAKLPVSPVDLSYLAPKNPGSGPAFTIINGTLKYFETRYIRVDISNPIIPHMVGTMSGTTTERELWND
WYPYEDVEIGPNGVLKTPTGFKFPLYMIGHGMLDSDLHKSSQAQVFEHPHAKDAASQLPDDETLFFGDTGLSKNP
VELVEGWFSSWKSTLASFFLIIGLGVALIFIIRIIVAIRYKYKGRKTQKIYNDVEMSRLGNK
VSV-G Marraba ectodomain WT:
(SEQ ID NO: 13)
KFTIVFPHHQKGNWKNVPSTYHYCPSSSDQNWHNDLTGVSLHVKIPKSHKAIQADGWMCHAAKWVTTCDFRWYGP
KYITHSIHSMSPTLEQCKTSIEQTKQGVWINPGFPPQSCGYATVTDAEVVVVQATPHHVLVDEYTGEWIDSQLVG
GKCSKEVCQTVHNSTVWHADYKITGLCESNLASVDITFFSEDGQKTSLGKPNTGFRSNHFAYESGEKACRMQYCT
QWGIRLPSGVWFELVDKDLFQAAKLPECPRGSSISAPSQTSVDVSLIQDVERILDYSLCQETWSKIRAKLPVSPV
DLSYLAPKNPGSGPAFTIINGTLKYFETRYIRVDISNPIIPHMVGTMSGTTTERELWNDWYPYEDVEIGPNGVLK
TPTGFKFPLYMIGHGMLDSDLHKSSQAQVFEHPHAKDAASQLPDDETLFFGDTGLSKNPVELVEGWFSSWKSTLA
SFFLIIGLGVALIFIIRIIVAIRYKYKGRKTQKIYNDVEMSRLGNK
VSV-G Carajas Full length WT:
(SEQ ID NO: 14)
MKMKMVIAGLILCIGILPAIGKITISFPQSLKGDWRPVPKGYNYCPTSADKNLHGDLIDIGLRLRAPKSFKGISA
DGWMCHAARWITTCDFRWYGPKYITHSIHSFRPSNDQCKEAIRLINEGNWINPGFPPQSCGYASVTDSESVVVTV
TKHQVLVDEYSGSWIDSQFPGGSCTSPICDTVHNSTLWHADHTLDSICDQEFVAMDAVLFTESGKFEEFGKPNSG
IRSNYFPYESLKDVCQMDFCKRKGFKLPSGVWFEIEDAEKSHKAQVELKIKRCPHGAVISAPNQNAADINLIMDV
ERILDYSLCQATWSKIQNKEALTPIDISYLGPKNPGPGPAFTIINGTLHYFNTRYIRVDIAGPVTKEITGFVSGT
STSRVLWDQWFPYGENSIGPNGLLKTASGYKYPLFMVGTGVLDADIHKLGEATVIEHPHAKEAQKVVDDSEVIFF
GDTGVSKNPVEVVEGWFSGWRSSLMSIFGIILLIVCLVLIVRILIALKYCCVRHKKRTIYKEDLEMGRIPRRA
VSV-G Carajas ectodomain WT:
(SEQ ID NO: 15)
KITISFPQSLKGDWRPVPKGYNYCPTSADKNLHGDLIDIGLRLRAPKSFKGISADGWMCHAARWITTCDFRWYGP
KYITHSIHSFRPSNDQCKEAIRLINEGNWINPGFPPQSCGYASVTDSESVVVTVTKHQVLVDEYSGSWIDSQFPG
GSCTSPICDTVHNSTLWHADHTLDSICDQEFVAMDAVLFTESGKFEEFGKPNSGIRSNYFPYESLKDVCQMDFCK
RKGFKLPSGVWFEIEDAEKSHKAQVELKIKRCPHGAVISAPNQNAADINLIMDVERILDYSLCQATWSKIQNKEA
LTPIDISYLGPKNPGPGPAFTIINGTLHYENTRYIRVDIAGPVTKEITGFVSGTSTSRVLWDQWFPYGENSIGPN
GLLKTASGYKYPLFMVGTGVLDADIHKLGEATVIEHPHAKEAQKVVDDSEVIFFGDTGVSKNPVEVVEGWFSGWR
SSLMSIFGIILLIVCLVLIVRILIALKYCCVRHKKRTIYKEDLEMGRIPRRA
VSV-G Alagoa Full length WT:
(SEQ ID NO: 16)
MTPAFILCMLLAGSSWAKFTIVFPQSQKGDWKDVPPNYRYCPSSADQNWHGDLLGVNIRAKMPKVHKAIKADGWM
CHAAKWVTTCDYRWYGPQYITHSIHSFIPTKAQCEESIKQTKEGVWINPGFPPKNCGYASVSDAESIIVQATAHS
VMIDEYSGDWLDSQFPTGRCTGSTCETIHNSTLWYADYQVTGLCDSALVSTEVTFYSEDGLMTSIGRQNTGYRSN
YFPYEKGAAACRMKYCTHEGIRLPSGVWFEMVDKELLESVQMPECPAGLTISAPTQTSVDVSLILDVERMLDYSL
CQETWSKVHSGLPISPVDLGYIAPKNPGAGPAFTIVNGTLKYFDTRYLRIDIEGPVLKKMTGKVSGTPTKRELWT
EWFPYDDVEIGPNGVLKTPEGYKFPLYMIGHGLLDSDLQKTSQAEVFHHPQIAEAVQKLPDDETLFFGDTGISKN
PVEVIEGWFSNWRSSVMAIVFAILLLVITVLMVRLCVAFRHFCCQKRHKIYNDLEMNQLRR
VSV-G Alagoa ectodomain WT:
(SEQ ID NO: 17)
KFTIVFPQSQKGDWKDVPPNYRYCPSSADQNWHGDLLGVNIRAKMPKVHKAIKADGWMCHAAKWVTTCDYRWYGP
QYITHSIHSFIPTKAQCEESIKQTKEGVWINPGFPPKNCGYASVSDAESIIVQATAHSVMIDEYSGDWLDSQFPT
GRCTGSTCETIHNSTLWYADYQVTGLCDSALVSTEVTFYSEDGLMTSIGRQNTGYRSNYFPYEKGAAACRMKYCT
HEGIRLPSGVWFEMVDKELLESVQMPECPAGLTISAPTQTSVDVSLILDVERMLDYSLCQETWSKVHSGLPISPV
DLGYIAPKNPGAGPAFTIVNGTLKYFDTRYLRIDIEGPVLKKMTGKVSGTPTKRELWTEWFPYDDVEIGPNGVLK
TPEGYKFPLYMIGHGLLDSDLQKTSQAEVFHHPQIAEAVQKLPDDETLFFGDTGISKNPVEVIEGWFSNWRSSVM
AIVFAILLLVITVLMVRLCVAFRHFCCQKRHKIYNDLEMNQLRR
VSV-G Cocal Full length WT:
(SEQ ID NO: 18)
MNFLLLTFIVLPLCSHAKFSIVFPQSQKGNWKNVPSSYHYCPSSSDQNWHNDLLGITMKVKMPKTHKAIQADGWM
CHAAKWITTCDFRWYGPKYITHSIHSIQPTSEQCKESIKQTKQGTWMSPGFPPQNCGYATVTDSVAVVVQATPHH
VLVDEYTGEWIDSQFPNGKCETEECETVHNSTVWYSDYKVTGLCDATLVDTEITFFSEDGKKESIGKPNTGYRSN
YFAYEKGDKVCKMNYCKHAGVRLPSGVWFEFVDQDVYAAAKLPECPVGATISAPTQTSVDVSLILDVERILDYSL
CQETWSKIRSKQPVSPVDLSYLAPKNPGTGPAFTIINGTLKYFETRYIRIDIDNPIISKMVGKISGSQTERELWT
EWFPYEGVEIGPNGILKTPTGYKFPLFMIGHGMLDSDLHKTSQAEVFEHPHLAEAPKQLPEEETLFFGDTGISKN
PVELIEGWFSSWKSTVVTFFFAIGVFILLYVVARIVIAVRYRYQGSNNKRIYNDIEMSRFRK
VSV-G Cocal ectodomain WT:
(SEQ ID NO: 19)
KFSIVFPQSQKGNWKNVPSSYHYCPSSSDQNWHNDLLGITMKVKMPKTHKAIQADGWMCHAAKWITTCDFRWYGP
KYITHSIHSIQPTSEQCKESIKQTKQGTWMSPGFPPQNCGYATVTDSVAVVVQATPHHVLVDEYTGEWIDSQFPN
GKCETEECETVHNSTVWYSDYKVTGLCDATLVDTEITFFSEDGKKESIGKPNTGYRSNYFAYEKGDKVCKMNYCK
HAGVRLPSGVWFEFVDQDVYAAAKLPECPVGATISAPTQTSVDVSLILDVERILDYSLCQETWSKIRSKQPVSPV
DLSYLAPKNPGTGPAFTIINGTLKYFETRYIRIDIDNPIISKMVGKISGSQTERELWTEWFPYEGVEIGPNGILK
TPTGYKFPLFMIGHGMLDSDLHKTSQAEVFEHPHLAEAPKQLPEEETLFFGDTGISKNPVELIEGWFSSWKSTVV
TFFFAIGVFILLYVVARIVIAVRYRYQGSNNKRIYNDIEMSRFRK
VSV-G Morreton Full length WT:
(SEQ ID NO: 20)
MLVLYLLLSLLALGAQCKFTIVFPHNQKGNWKNVPANYQYCPSSSDLNWHNGLIGTSLQVKMPKSHKAIQADGWM
CHAAKWVTTCDFRWYGPKYVTHSIKSMIPTVDQCKESIAQTKQGTWLNPGFPPQSCGYASVTDAEAVIVKATPHQ
VLVDEYTGEWVDSQFPTGKCNKDICPTVHNSTTWHSDYKVTGLCDANLISMDITFFSEDGKLTSLGKEGTGFRSN
YFAYENGDKACRMQYCKHWGVRLPSGVWFEMADKDIYNDAKFPDCPEGSSIAAPSQTSVDVSLIQDVERILDYSL
CQETWSKIRAHLPISPVDLSYLSPKNPGTGPAFTIINGTLKYFETRYIRVDIAGPIIPQMRGVISGTTTERELWT
DWYPYEDVEIGPNGVLKTATGYKFPLYMIGHGMLDSDLHISSKAQVFEHPHIQDAASQLPDDETLFFGDTGLSKN
PIELVEGWFSGWKSTIASFFFIIGLVIGLYLVLRIGIALCIKCRVQEKRPKIYTDVEMNRLDR
VSV-G Morreton ectodomain WT:
(SEQ ID NO: 21)
KFTIVFPHNQKGNWKNVPANYQYCPSSSDLNWHNGLIGTSLQVKMPKSHKAIQADGWMCHAAKWVTTCDFRWYGP
KYVTHSIKSMIPTVDQCKESIAQTKQGTWLNPGFPPQSCGYASVTDAEAVIVKATPHQVLVDEYTGEWVDSQFPT
GKCNKDICPTVHNSTTWHSDYKVTGLCDANLISMDITFFSEDGKLTSLGKEGTGFRSNYFAYENGDKACRMQYCK
HWGVRLPSGVWFEMADKDIYNDAKFPDCPEGSSIAAPSQTSVDVSLIQDVERILDYSLCQETWSKIRAHLPISPV
DLSYLSPKNPGTGPAFTIINGTLKYFETRYIRVDIAGPIIPQMRGVISGTTTERELWTDWYPYEDVEIGPNGVLK
TATGYKFPLYMIGHGMLDSDLHISSKAQVFEHPHIQDAASQLPDDETLFFGDTGLSKNPIELVEGWFSGWKSTIA
SFFFIIGLVIGLYLVLRIGIALCIKCRVQEKRPKIYTDVEMNRLDR

What is claimed:

1. A pharmaceutical composition comprising:
(i) a lentiviral vector;
(ii) a pharmaceutically acceptable buffer at a concentration of about 5 mM to about 100 mM;
(iii) a pharmaceutically acceptable salt at a concentration of about 100 mM to about 800 mM;
(iv) a sugar in an amount of about 1% (w/v) to about 5% (w/v); and
(v) a protein excipient in an amount of about 0.1% (w/v) to about 3% (w/v),
wherein the pharmaceutical composition has a pH of about 6.0 to about 8.0.

2. The pharmaceutical composition of claim 1, wherein the lentiviral vector is present at a concentration of about 1.0E⁺⁰⁵ to about 1.0E⁺⁰⁹ TUs/mL.

3. The pharmaceutical composition of claim 1 or 2, wherein the pharmaceutically acceptable buffer is histidine buffer, HEPES, phosphoric acid buffer, citric acid buffer, acetic acid buffer, succinic acid buffer, phosphate buffer, acetate buffer, citrate buffer, succinate buffer, ascorbic acid buffer, glutamic acid buffer, lactic acid buffer, maleic acid buffer, trometamol buffer, or gluconic acid buffer, or any combination thereof.

4. The pharmaceutical composition of any one of claims 1-3, wherein the pharmaceutically acceptable buffer is a histidine buffer.

5. The pharmaceutical composition of claim 4, wherein the histidine buffer is present at a concentration of about 10 mM to about 30 mM.

6. The pharmaceutical composition of any one of claims 1-5, wherein the pharmaceutical composition has a pH of about 6.5 to about 7.5.

7. The pharmaceutical composition of any one of claims 1-6, wherein the pharmaceutically acceptable salt is sodium chloride, potassium chloride, magnesium chloride, or calcium chloride, or any combination thereof.

8. The pharmaceutical composition of any one of claims 1-7, wherein the pharmaceutically acceptable salt is sodium chloride.

9. The pharmaceutical composition of claim 8, wherein the sodium chloride is present at a concentration of about 100 mM to about 250 mM.

10. The pharmaceutical composition of any one of claims 1-9, wherein the sugar is sucrose or trehalose, or a combination thereof.

11. The pharmaceutical composition of any one of claims 1-10, wherein the sugar is sucrose.

12. The pharmaceutical composition of claim 11, wherein the sucrose is present at about 2% (w/v) to about 4% (w/v).

13. The pharmaceutical composition of any one of claims 1-12, wherein the protein excipient is recombinant human serum albumin, non-recombinant human serum albumin, or a combination thereof.

14. The pharmaceutical composition of any one of claims 1-13, wherein the protein excipient is non-recombinant human serum albumin.

15. The pharmaceutical composition of claim 13 or 14, wherein the non-recombinant human serum albumin is USP grade non-recombinant human serum albumin.

16. The pharmaceutical composition of any one of claims 13-15, wherein the non-recombinant human serum albumin is present at an amount of about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3.0% (w/v).

17. The pharmaceutical composition of any one of claims 1-16, wherein the lentiviral vector is a VSV-G pseudotyped lentivirus.

18. The pharmaceutical composition of claim 17, wherein the VSV-G pseudotyped lentivirus comprises a VSV-G polypeptide comprising a mutation that corresponds to a mutation at position 182 of SEQ ID NO: 2.

19. A pharmaceutical composition comprising:
(i) a VSV-G pseudotyped lentivirus
(ii) about 10 mM to about 30 mM of a L-Histidine buffer;
(iii) about 100 mM to about 400 mM of sodium chloride;
(iv) about 1% (w/v) to about 5% (w/v) of sucrose; and
(v) about 0.1% (w/v) to about 2% (w/v) of a non-recombinant human serum albumin,
wherein the pharmaceutical composition has a pH of about 5.5 to about 8.0.

20. The pharmaceutical composition of claim 19, wherein the VSV-G pseudotyped lentivirus is present at a concentration of about 1.0E⁺⁰⁵ to 1.0E⁺⁰⁹ TUs/mL.

21. The pharmaceutical composition of any one of claims 19-20, wherein the L-Histidine is present at a concentration of about 15-25 mM and the pharmaceutical composition has a pH of about 6 to about 7.

22. The pharmaceutical composition of any one of claims 19-21, wherein sodium chloride is present at a concentration of about 125 mM to about 175 mM.

23. The pharmaceutical composition of any one of claims 19-22, wherein sucrose is present in an amount of about 2% (w/v) to about 5% (w/v).

24. The pharmaceutical composition of any one of claims 19-23, wherein the non-recombinant human serum albumin is present at about 0.1% (w/v) to about 1.5% (w/v).

25. The pharmaceutical composition of any one of claims 19-24, wherein the non-recombinant human serum albumin is USP grade non-recombinant human serum albumin.

26. The pharmaceutical composition of any one of claims 19-25, wherein the VSV-G pseudotyped lentivirus comprises a VSV-G polypeptide comprising a mutation that corresponds to a mutation at position 182 of SEQ ID NO: 2.

27. The pharmaceutical composition of claim 26, wherein the VSV-G polypeptide comprises a I182A, I182D, or I182E mutation as compared to SEQ ID NO: 2.

28. The pharmaceutical composition of claim 26 or 27, wherein the VSV-G polypeptide comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to a sequence of SEQ ID NO: 4.

29. The pharmaceutical composition of claim 26 or 27, wherein the VSV-G polypeptide comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to a sequence of SEQ ID NO: 5.

30. The pharmaceutical composition of any one of claims 26-29, wherein the VSV-G polypeptide, further comprises a mutation that corresponds to a mutation at positions 214 and/or 352 of SEQ ID NO: 2.

31. The pharmaceutical composition of claim 30, wherein the VSV-G polypeptide comprises substitutions at positions I182, T214, and T352 of SEQ ID NO: 2.

32. The pharmaceutical composition of claim 30 or 31, wherein the substitution at position 182 of the VSV-G polypeptide is I182D or I182E, the substitution at position 214 of the VSV-G polypeptide is T214N, and the substitution at position 352 of the VSV-G polypeptide is T352A.

33. The pharmaceutical composition of any one of claims 30-32, wherein the VSV-G polypeptide, comprises a sequence of SEQ ID NO: 22, SEQ ID NO: 23, SEQ ID NO: 24 or SEQ ID NO: 25.

34. A pharmaceutical composition comprising:
(i) a VSV-G pseudotyped lentivirus comprising a VSV-G polypeptide comprising a mutation that corresponds to a mutation at position 182, 214, and/or 352 of SEQ ID NO: 2;
(ii) L-Histidine buffer at a concentration of about 10 mM to about 30 mM;
(iii) sodium chloride at a concentration of about 100 mM to about 400 mM;
(iv) sucrose in amount of about 1% (w/v) to about 5% (w/v); and
(v) non-recombinant human serum albumin in an amount of about 0.1% (w/v) to about 2% (w/v),
wherein the pharmaceutical composition has a pH of about 6.5 to about 7.5.

35. The pharmaceutical composition of any one of claims 1-34, wherein the lentiviral vector further comprises a targeting moiety.

36. The pharmaceutical composition of claim 35, wherein the targeting moiety binds to CD7.

37. The pharmaceutical composition of claim 36, wherein the targeting moiety comprises a polypeptide comprising: (i) a heavy chain variable region comprising heavy chain CDR1, CDR2, and CDR3 sequences, wherein the heavy chain CDR1 sequence has the amino acid sequence of SEQ ID NO: 30; the heavy chain CDR2 has the amino acid sequence of SEQ ID NO: 31; and the heavy chain CDR3 sequence has the amino acid sequence of SEQ ID NO: 32, or variants of any of the foregoing; and (ii) a light chain variable region comprising light chain CDR1, CDR2, and CDR3 sequences, wherein the light chain CDR1 sequence has the amino acid sequence of SEQ ID NO: 33; the light chain CDR2 sequence has the amino acid sequence of SEQ ID NO: 34; and the light chain CDR3 sequence has the amino acid sequence of SEQ ID NO: 35; or variants of any of the foregoing.

38. The pharmaceutical composition of claim 36 or 37, wherein the targeting moiety comprises a heavy chain variable region (VH) comprising an amino acid sequence of SEQ ID NO: 42 and a light chain variable region (VL) comprising an amino acid sequence of SEQ ID NO: 43.

39. The pharmaceutical composition of any one of claims 36-38, wherein the targeting moiety comprises an antigen binding domain comprising an amino acid sequence of SEQ ID NO: 44 or SEQ ID NO: 45.

40. The pharmaceutical composition of any one of claims 35-39, wherein the targeting moiety comprises a formula of T-S₁, wherein T is a target binding domain and S₁ is a stalk portion, wherein the targeting moiety is attached to the surface of the viral vector through a stalk portion, S₁.

41. The pharmaceutical composition of claim 40, wherein the stalk portion, S1, comprises a formula of L₁-Fc-L₂-X₁, wherein:
L₁ is a linker or absent;
Fc is a variant Fc protein;
L₂ is a linker or absent; and
X₁ is a polypeptide comprising the transmembrane domain,
wherein the targeting moiety having the formula T-S₁ has a formula of T-L₁-Fc-L₂-X₁.

42. The pharmaceutical composition of claim 41, wherein X₁ comprises a polypeptide having a formula of ECD-T_{M}-ICD, wherein:
ECD is an extracellular domain, or a fragment thereof, of a cell surface protein, or absent;
T_{M} is a transmembrane domain of a transmembrane protein; and
ICD is an intracellular domain or a protein that facilitates incorporation of the targeting moiety into the envelope of the viral particle, or absent,
wherein the targeting moiety having the formula of T-L₁-Fc-L₂-X₁ has a formula of T-L₁-Fc-L₂-ECD-T_{M}-ICD.

43. The pharmaceutical composition of claim 42, wherein:
T comprises an amino acid sequence of SEQ ID NO: 44, SEQ ID NO: 45, SEQ ID NO: 62, or SEQ ID NO: 63;
L₁ comprises the amino acid sequence of SEQ ID NO: 72;
Fc comprises the amino acid sequence of SEQ ID NO: 82;
L₂ is absent;
ECD comprises the amino acid sequence of SEQ ID NO: 83;
T_{M} comprises the amino acid sequence of SEQ ID NO: 84; and
ICD comprises an amino acid sequence comprising an env incorporation motif,
wherein the env incorporation motif comprises an amino acid sequence of SEQ ID NO: 85.

44. The pharmaceutical composition of claim 40, wherein the targeting moiety comprising a formula of T-S₁ comprises an amino acid sequence having at least 90% identity to SEQ ID NO: 89.

45. The pharmaceutical composition of any one of claims 1-44, wherein the lentiviral vector further comprises a nucleic acid molecule encoding for a heterologous molecule of interest.

46. The pharmaceutical composition of claim 45, wherein the heterologous molecule of interest is a chimeric antigen receptor (“CAR”).

47. The pharmaceutical composition of claim 46, wherein the CAR comprises an antigen binding domain comprising an antibody, or a fragment thereof.

48. The pharmaceutical composition of claim 47, wherein the antigen binding domain of the CAR binds to CD20.

49. The pharmaceutical composition of claim 48, wherein the antigen binding domain that binds to CD20 comprises a polypeptide comprising a light chain and a heavy chain comprising: a heavy chain variable region of the heavy chain having at least 90% identity to an amino acid sequence of SEQ ID NO: 73; and a light chain variable region of the light chain having at least 90% identity to an amino acid sequence of SEQ ID NO: 74.

50. The pharmaceutical composition of claim 48 or 49, wherein the antigen binding domain that binds to CD20 comprises an amino acid sequence of SEQ ID NO: 75 or SEQ ID NO: 76.

51. The pharmaceutical composition of any one of claims 48-50, wherein the CAR comprises an amino acid sequence having at least 90% identity to SEQ ID NO: 90.

52. A method of infecting a cell, the method comprising contacting the cell with the pharmaceutical composition of any one of claims 1-51.

53. A method of delivering a heterologous molecule of interest to a cell, the method comprising contacting the cell with the pharmaceutical composition of any one of claims 1-51, wherein the lentiviral vector of the pharmaceutical composition comprises a nucleic acid molecule encoding the heterologous molecule of interest.

54. A method of delivering a heterologous molecule of interest to a subject, the method comprising administering to the subject the viral particle of any one of claims 1-51, wherein the lentiviral vector of the pharmaceutical composition comprises a nucleic acid molecule encoding the heterologous molecule of interest.

55. A method of treating a disease or disorder in a subject, the method comprising administering to the subject the pharmaceutical composition of any one of claims 1-51, wherein the lentiviral vector of the pharmaceutical composition comprises a nucleic acid molecule encoding a heterologous molecule of interest to treat the disease or disorder.

56. A method of delivering a heterologous molecule to a target cell, the method comprising contacting the cell with the pharmaceutical composition of any one of claims 1-51, wherein the lentiviral vector of the pharmaceutical composition comprises a nucleic acid molecule encoding the heterologous molecule.

57. The pharmaceutical composition of any one of claims 1-51, for use in the treatment of a cancer, an immune disorder, an auto-immune disorder, or a metabolic disorder.

58. Use of the pharmaceutical composition of any one of claims 1-51, for the manufacture of a medicament for treatment of a cancer, an immune disorder, an auto-immune disorder, or a metabolic disorder.
